(19) United States
(12) Patent Application Publication
(10) Pub. No.: US 2005/0112706 A1
(43) Pub. Date: May 26, 2005

(54) Diagnostic and prognostic methods for prostate cancers

(76) Inventor: Susan Kasper, Nashville, TN (US)

Correspondence Address: FULBRIGHT & JAWORSKI L.L.P., 600 CONGRESS AVE., SUITE 2400, AUSTIN, TX, 78701, US

(21) Appl. No.: 10/703,209

(22) Filed: Nov. 6, 2003

Related U.S. Application Data
(60) Provisional application No. 60/424,490, filed on Nov. 7, 2002.

(51) Int. Cl. G01N 33/53; G01N 33/574; G01N 33/20

(52) U.S. Cl. 435/7.23; 436/84

(57) ABSTRACT

The invention includes prognostic and diagnostic methods and compositions for characterizing disease states, biopsy samples, biological samples, cells, and tissues. In various embodiments of the invention, a bioassay may be used to analyze the activity of the androgen receptor (AR) in human prostate epithelial (HPE) cells derived from patient samples. The activity of the AR may be used to characterize the sample in regard to AR activity. Characterization of a sample may be useful in classify the sample and/or in determining a treatment regiment. In other embodiments, a sample may be compared to other known samples to identify other distinguishing characteristics, such as disease markers, and/or determine a treatment regiment based on the comparison of samples.

The present application claims priority to co-pending U.S. Patent Application Ser. No. 60/424,490 filed Nov. 7, 2002. The entire text of the above-referenced disclosure is specifically incorporated by reference herein without disclaimer.

The government owns rights in the present invention pursuant to grant number DK060957 from the National Institutes of Health.

### BACKGROUND OF THE INVENTION

1. Field of the Invention

The present invention relates generally to the fields of oncolology. More particularly, it concerns diagnostic and prognostic methods for identification or determination of hormone responsiveness of a tumor cell, tissue, or sample.

2. Description of Related Art

The initial treatment for localized prostate cancer is usually surgery or radiation treatment. Unfortunately, not all patients are cured by this therapy and their disease may recur when prostate cancer cells, which have escaped the prostatic capsule, continue to proliferate and metastasize. In other cases, prostate cancer is initially diagnosed only after the disease has already advanced and spread. Since androgens are the primary growth factor for prostate cells, a commonly used approach to the management of advanced prostate cancer is the chemical or surgical removal of androgens. The adrenal glands produce up to 5% of serum androgens and therefore, antiandrogens such as flutamide or bicalutamide may be added to the treatment regime to suppress adrenal androgen production in what is referred to as maximum androgen blockade (MAB). Subsequently, regression is followed by monitoring the decline of serum PSA levels and the maintenance of nadir PSA levels. Subsequent rise of those levels is an indicator of prostate cancer progression. Although 85-90% of patients with advanced prostate cancer initially respond to this therapy, men with metastatic disease eventually develop androgen-independent prostate cancer (AIPC).

The withdrawal of steroid hormones is the first line of treatment after primary hormonal therapy failure (Kelly et al., 1997). Kelly et al. observed that in 30% of cases, serum PSA levels declined after hormonal withdrawal. Other parameters such as acid phosphatase and alkaline phosphatase levels also decreased. In some cases, the patients' hemoglobin levels rose and repeat CT scans showed a significant regression in metastatic liver lesions. The pathophysiology of the antiandrogen withdrawal syndrome is poorly understood. However, it is thought that the androgen receptor (AR) plays a major role in the development of AIPC (reviews Grossmann et al., 2001 and Feldman and Feldman, 2001). Currently, it is not possible to identify the subset of patients whose tumors will respond to steroid hormone and antiandrogen withdrawal. Thus, cessation of flutamide treatment for at least 4 weeks and bicalutamide treatment for up to 8 weeks is necessary to exclude the antiandrogen withdrawal syndrome as the cause of increasing PSA values (Paul and Breul, 2000).

The observation that about 30% of patients respond to steroid hormone and antiandrogen withdrawal suggests that the AR is still central in the progression to AIPC. Androgens are initially required for the development of the normal prostate and androgens can both stimulate growth and inhibit apoptosis of prostate cancer cells. Prostate cancer cells may become hormone refractory through several different mechanisms: a) AR mutations in the hormone binding domain, or increased AR levels through amplification of the AR gene, which increase the sensitivity of AR to low serum androgen levels, b) AR mutations which allow activation by antiandrogens and other steroid hormones, c) alterations in the interaction of AR with its co-regulators which activate AR in an aberrant manner, d) proteins, such as growth factors and cytokines, from other signaling pathways which activate AR in the absence of androgen and e) prostate cancer cells which do not require AR or androgens to metastasize.

There is no standard treatment of AIPC. Knowing the effects of androgen and antiandrogen treatment on human prostate epithelial (HPE) cells derived from individual patient samples can identify mechanisms by which prostate cancer cells progress from androgen-dependent to androgen-independent cell growth. However, such a test is not currently available.

### SUMMARY OF THE INVENTION

The invention includes prognostic and diagnostic methods and compositions for characterizing disease states, biopsy samples, biological samples, cells, and tissues. In various embodiments of the invention, a bioassay may be used to analyze the activity of the androgen receptor (AR) in human prostate epithelial (HPE) cells derived from patient samples. The activity of the AR may be used to characterize the sample in regard to AR activity. Characterization of a sample may be useful in classify the sample and/or in determining a treatment regiment. In other embodiments, a sample may be compared to other known samples to identify other distinguishing characteristics, such as disease markers, and/or determine a treatment regiment based on the comparison of samples.

Certain embodiments of the invention include methods for determining androgen-responsiveness of a first biological sample from a patient suspected of having cancer, comprising: (a) producing a protein profile of the first biological sample derived from the patient suspected of having cancer; (b) comparing the protein profile of the first biological sample with the protein profile of a second biological sample that has been characterized as being androgen-responsive and a third biological sample that has been characterized as being androgen-independent; and (d) determining if the first biological sample is androgen-responsive or androgen-independent based on the comparison of the protein profiles.

Androgen responsiveness (i.e., “androgen-responsive” or “androgen-independent”) is defined as a measure of the ability of a cell or tissue to respond physiologically to the presence of an androgen, particularly through the activation of the androgen receptor and its downstream affects. A protein profile is defined as a qualitative or quantitative characterization of all or part of a protein population in a given sample. Methods of protein profiling include but are not limited to chromatographic methods, such as high pressure liquid chromatography; Mass spectral methods, such as MALDI mass spectrometry; or electrophoretic methods such as 2-D gel electrophoresis. The term cancer includes, but is not limited to all androgen sensitive cancers, such as prostate or testicular cancer. A biological sample may be obtained using known techniques. For example a needle or punch biopsy and may also include collection of tissue as a result of resection of a tumor.

In other embodiments of the invention, methods for determining a cancer treatment for a patient suspected of having cancer are contemplated. The methods may include: (a) producing a protein profile of a first biological sample from the patient; (b) comparing the protein profile of the first biological sample with the protein profiles of a set of biological samples for which androgen-responsiveness has been determined; and (d) determining a treatment for the patient based on whether the first biological sample is androgen-responsive. The cancer may be any androgen sensitive cancer, for example prostate cancer. Protein profiles and biopsies may be obtained by methods described above.

In certain embodiments, androgen-responsiveness, as determined by a methods described herein, may be determined by methods including (a) transfecting epithelial cells of the biological sample with an androgen-regulatable expression construct; (b) exposing the transfected cells to an androgen; (c) assaying levels of expression from the androgen-regulatable expression construct in the androgen exposed transfected cells relative to a control; and (d) determining androgen-responsiveness of the biological sample. The androgen-regulatable expression construct may comprise an androgen-regulated promoter element operatively linked to a reporter gene.

In various embodiments an androgen regulated promoter element is an androgen receptor binding site 1 (ARBS-1) and androgen receptor binding site 2 (ARBS-2) of a probasin gene or any other known androgen regulatable promoter element. A reporter gene may be operatively linked to an androgen regulatable promoter element. Reporter genes may include, but are not limited to chloramphenicol acetyltransferase (CAT), luciferase, green fluorescent protein, horse radish peroxidase, β-galactosidase, and other known reporter genes. Methods for assaying levels of reporter gene expression include, but are not limited to nucleic acid blotting, western blotting, enzymatic assay or spectrometry.

An androgen-regulatable expression construct may be a plasmid, viral, linear, or circular expression construct. In particular embodiments the expression vector is a viral expression construct. Viral expression constructs include, but are not limited to adenovirus, retrovirus, AAV, lentivirus, herpesvirus and other known viral expression vectors. Transfection of expression constructs may be accomplished through any means that allows the expression construct to be introduced into a tissue or cell. Transfection includes infection by a virus.

In other embodiments of the invention, methods for characterizing androgen-responsiveness of a biological sample are contemplated. The methods may include (a) transfecting epithelial cells of the biological sample with an androgen-regulatable expression construct; (b) exposing the transfected cells to an androgen; (c) assaying levels of expression from the androgen-regulatable expression construct in the androgen exposed transfected cells relative to a control; and (d) determining androgen-responsiveness of the biological sample. Androgen regulatable expression constructs and promoters, reporter genes and assay methods, as well as other methodologies are as described above and herein.

In certain embodiments, methods include determining an appropriate treatment for prostate cancer in a patient suspected of having prostate cancer comprising: (a) transfecting epithelial cells of a biological sample from the patient suspected of having prostate cancer with an androgen-regulatable expression construct, wherein the expression construct comprises an androgen-responsive promoter element operatively linked to a reporter gene; (b) exposing the transfected cells to an androgen; (c) exposing the transfected cells to an antiandrogen; (d) assaying levels of reporter gene expression in the transfected cells of (b) and (c) relative to a control; (e) determining an appropriate treatment for the patient. Androgen regulatable expression constructs and promoters, reporter genes and assay methods, as well as other methodologies are as described above and herein.

In various embodiments, a method may include determining androgen-responsiveness of a biological sample from a patient, comprising: (a) producing a protein profile of the first biological sample; (b) producing protein profiles of a set of biological samples characterized for androgen-responsiveness by a method comprising (i) transfecting epithelial cells of a biological sample with an androgen-regulatable expression construct, (ii) exposing the transfected cells to an androgen or antiandrogen treatment; and (iii) detecting levels of reporter gene expression in the treated transfected cells relative to a control; (c) comparing the protein profile of the first biological sample with the protein profiles of the set of biological samples for which androgen-responsiveness has been determined; and (d) determining the androgen-responsiveness of the first biological sample. The set of biological samples are derived from various samples with a known androgen responsiveness and a particular protein profile, akin to a set of standards. Androgen regulatable expression constructs and promoters, reporter genes and assay methods, as well as other methodologies, such as protein profiles, biopsies, androgen responsiveness are as described above and herein.

In other embodiments, methods include identifying a protein marker for androgen-responsive prostate cancer comprising: (a) transfecting epithelial cells of a first biological sample with an androgen-regulatable expression construct, wherein the expression construct comprises an androgen-responsive promoter element operatively linked to a reporter gene; (b) exposing the transfected cells to an androgen; (c) detecting levels of reporter gene expression in the treated transfected cells relative to a control; (d) determining androgen-responsiveness of the first biological sample; (e) producing a protein profile of an androgen-responsive biological sample; (f) comparing the protein profile of the androgen-responsive biological sample to a protein profile of a second androgen-independent biological sample; and (g) identifying a protein that is diagnostic of androgen-responsive cancer. Based on a difference in the androgen-responsive sample versus the androgen-independent sample. The cancer may be prostate cancer. A protein marker or marker protein is a protein that is associate with a particular condition and may be used as a diagnostic for that particular condition. Protein markers or fragments thereof may be utilized to produce antibodies that may be used in diagnostic, prognostic or therapeutic methods. Androgen regulatable expression constructs and promoters, reporter genes and assay methods, as well as other methodologies are as described above and herein.

In some embodiments, methods include identifying a protein marker for androgen-independent prostate cancer comprising: (a) transfecting epithelial cells of a biological sample with an androgen-regulatable expression construct, wherein the expression construct comprises an androgen-responsive promoter element operatively linked to a reporter gene; (b) exposing the transfected cells to an androgen; (c) detecting levels of reporter gene expression in the treated transfected cells relative to a control; (d) determining androgen-responsiveness of the biological sample; (e) producing a protein profile of an androgen-independent biological sample; (f) comparing the protein profile of the androgen-independent biological sample to a protein profile of a second androgen-responsive biological sample; and (g) identifying a protein that is diagnostic of an androgen-independent cancer. The cancer may be prostate cancer. Protein markers or fragments thereof may be utilized to produce antibodies that may be used in diagnostic, prognostic or therapeutic methods. Androgen regulatable expression constructs and promoters, reporter genes and assay methods, as well as other methodologies are as described above and herein.

In certain embodiments, a protein marker for an androgen-responsive prostate cancer is identified by a method comprising: (a) transfecting epithelial cells of a biological sample with an androgen-responsive expression construct, wherein the expression construct comprises an androgen-responsive promoter element operatively linked to a reporter gene; (b) exposing the transfected cells to an androgen, an antiandrogen, or an androgen and antiandrogen; (c) detecting levels of reporter gene expression in the transfected cells relative to a control; (d) producing a protein profile from transfected cells identified as being androgen-responsive; (e) determining the identity a protein that is diagnostic of an androgen-responsive epithelial cell.

In certain embodiments, a protein marker for an androgen-independent prostate cancer may be identified by a method comprising: (a) transfecting epithelial cells of a biological sample with an androgen-responsive expression construct, wherein the expression construct comprises an androgen-responsive promoter element operatively linked to a reporter gene; (b) exposing the transfected cells to an androgen, an antiandrogen, or an androgen and an antiandrogen; (c) detecting levels of reporter gene expression in the treated transfected cells relative to a control; (d) producing a protein profile from transfected cells identified as being androgen-independent; (e) determining the identity a protein that is diagnostic of an androgen-independent epithelial cell.

An androgen may be any compound that activates the AR by binding the AR ligand binding domain, e.g., testosterone, DHT, analogues thereof. An antiandrogen may be any compound that inhibits the activation of AR when bound to the AR, e.g., flutamide, finasteride and similar compounds. The androgen-regulatable expression construct includes an androgen-regulatable promoter element operatively linked to a reporter gene, which is a gene or transcript that may be assayed to determine the transcriptional activity of an expression construct. In certain embodiments, a probasin ARR2PB promoter may be linked to a choramphenicol acetyl transferase (CAT) or other known reporter gene, subcloned into nucleic acid delivery vector, for example an adenovirus (in particular the AdenoQuest™ AdBN adenoviral vector used to generate adenoviral particles).

HPE cells may be cultured out of patient samples, infected with the adenoviral particles and CAT gene activity can be measured in response to androgen (e.g., DHT or R1881) or antiandrogen (e.g., flutamide) treatment. Using embodiments of the assay described herein, the inventors have been able to measure the response of hormonal treatment or exposure at the transcriptional level. Typically, three primary subgroups (androgen profiles) of HPE cells have been identified: 1) those where androgen activated reporter gene expression and antiandrogen suppressed this activity, 2) those where androgen had little effect, but antiandrogen could still suppress reporter gene activity, and 3) those where antiandrogen acted as an agonist, enhancing reporter gene expression. The HPE bioassay demonstrates that prostate epithelial cells derived from patient biopsy samples respond to antiandrogen treatment in a similar manner as seen in patients undergoing androgen ablation therapy. Thus, the bioassay may be used to determine the sensitivity of a cancer to treatment regiment or to characterize a particular biological sample.

It is contemplated that any embodiment discussed with respect to one method of the invention may be implemented with respect to any other method of the invention.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-G. HPE cells retain a secretory phenotype. FIG. 1A. prostate punch biopsy fragment in organ culture. FIG. 1B. HPE cells in monolayer culture. FIG. 1C. Immunoperoxidase localization of AR shows marked accumulation of AR in the nucleus of most cells. The cytoplasm shows low levels of staining. FIG. 1D. Control for A. Anti-AR antibody co-incubated with AR oligopeptide fragment shows marked reduction in the level of staining. FIG. 1E. Immunoperoxidase localization of PSA shows granular cytoplasmic staining consistent with a secretory granule staining pattern. FIG. 1F. CD59 immunolocalization shows abundant extracellular, granular and intracellular granular staining consistent with prostasome pattern. FIG. 1G. Adipophilin immunoreactivity is localized in a vacuolar, intracellular compartment consistent with lipid droplets.

FIGS. 2A-E. Ultrastructural Analyses of HPE cells. HPE cells were analyzed by electron microscopy. FIG. 2A. large secretory body (**) filled with multiple tightly packed concentric membrane lamellae. FIG. 2B. Desmosomes (<) containing attachment plaques with tonofilaments radiating from these plagues, apical brush borders (BB) and brush border vesicle (→). FIG. 2C. Smaller secretory bodies (*) containing loosely organized membranes. FIG. 2D. Numerous secretory granules (*) released at the apical brush borders of HPE cells between 2 adjacent cells. FIG. 2E. HPE cells with large and smaller secretory bodies (magnification, 40×).

FIGS. 3A-C. HPE cells in short term culture maintain characteristics of prostate epithelial. FIG. 3A. the negative control for pan-cytokeratin was co-incubated with normal rabbit serum. FIG. 3B. Immunoperoxidase localization of CK shows marked accumulation of CK in cytoplasm. FIG. 3C. RT-PCR utilizing primers for CK8 and CK18. 18S served as an internal standard and cytokeratin-negative NIH 3T3 fibroblasts served as a negative control.

FIGS. 4A-C. Primary HPE cells express AR. HPE primary cultures from 7 different patients were analyzed for AR expression. FIG. 4A. Western blot analyses for AR. DU-145 prostate cancer cells served as a negative control. Thirty micrograms protein of HPE and DU145 samples were loaded per lane. For the LNCaP positive control, 0.5 μg/lane protein was loaded. FIG. 4B RT-PCR analyses for AR and PSA of the same samples as in A. DU-145 cells were negative controls for AR and PSA expression. Two μg total RNA were analyzed in each RT-PCR reaction. FIG. 4C. Real-time RT-PCR analyses of AR levels in HPE cells. LNCaP served as a positive control.

FIG. 5. Comparison of ARR2PB and PSE1.6 promoters. LNCAP cells were transfected with ARR2PB-CAT/AdBN and PSE1.6-CAT/AdBN and CAT activity was determined in response to 10⁻⁸M R1881. The insert is an expanded scale representing the results of PSE1.6-CAT/AdBN−/+10⁻⁸M R1881.

FIGS. 6A-B. Androgen and antiandrogen activity in LNCaP cells. LNCaP cells were infected with 10⁸ pfu ARR2PB-CAT/AdBN viral particles. FIG. 6A. Cells were treated with increasing concentrations of R1881 (10⁻¹² to 10⁻⁶M) to determine the effect of androgen treatment on CAT gene expression. FIG. 6B. LNCaP cells were treated with 10-8 M DHT and increasing concentrations of the antiandrogen flutamide (10⁻⁸ to 10⁻⁵M) to determine whether androgen-induced CAT expression could be inhibited by antiandrogen treatment.

FIGS. 7A-D. HPE bioassay determines AR function in HPE cells derived from human prostate biopsy samples. For the HPE bioassay, 1×10⁵ HPE cells/well were plated in quadruplicate in 24 well plates and the cells were infected with 10⁸ pfu ARR2PB-CAT/AdBN viral particles (AdPBCAT). The cells were treated with 10⁻⁸M R1881 or with 10⁻⁵M flutamide as indicated below each graph. CAT activity was determined as described previously (Kasper et al., 1994).

FIGS. 8A-C. Response of HPE cells cultured from different passages or from different prostate regions to androgen and antiandrogen treatment. HPE cells from passages 2 (P2) and 4 (P4) were plated in quadruplicate at 1×10⁵ HPE cells/well, infected with 10⁸ pfu ARR2PB-CAT/AdBN viral particles (AdPBCAT) and treated with 10⁻⁸M R1881 or with 10⁻⁵M flutamide as indicated below each graph. As well, HPE cells were cultured from the peripheral (PZ) and transitional (TZ) zones of the same prostate and treated with 10⁻⁸M R1881 or with 10⁻⁵M flutamide as indicated below each graph.

FIGS. 9A-B. Identification of androgen- and flutamide-regulated proteins by 2D difference gel electrophoresis. HPE cells were cultured and treated (−) hormone, (+) 10 M DHT or (+) 10⁻⁵ M flutamide. Whole cell proteins were extracted, labeled with the dyes Cy2 (ex 480, em 530), Cy3 (ex 540, em 590), and Cy5 (ex 620, em 680) respectively. The labeled proteins were mixed together and subjected to a pH gradient of pH 4 to pH 7 in the first dimension and separated by PAGE in the second dimension. The gel was analyzed under the appropriate three wave lengths for proteins that were either up-regulated or down-regulated in response to hormonal treatment, and subsequently stained with Sypro Ruby Red dye to aid in excising the relevant spots. The proteins were excised, subjected to in-gel tryptic digestion and analyzed by MS. The tryptic peptides were identified by searching the protein data bases such as the NCBInr and SWISS- PROT databases. EST databases were also searched with fragmentation data from tandem mass spectrometry when necessary.

FIG. 10. Correlation of protein profiles obtained from frozen tissue sections to the response of HPE cells to androgen ablation therapy. Ten micron sections were cut from four different patient biopsies, spotted with matrix and subjected to MS imaging analyses. The protein profiles were then compared and grouped according to pattern similarity. Biopsy 1839-1 was similar to biopsy 3771-1, but with fewer differences in the protein peaks. Biopsy 3771-1 had the greatest number of differences from all 4 samples and was considered the “most interesting.” Profiles from biopsies 1990-1 and 2CaP-1 were nearly identical (with only 2 obvious differences), even though they originated from 2 different patient biopsies. When the protein profiles were compared to the results from the HPE bioassay, reporter gene activity in HPE cells from biopsy 1839-1 increased in response to androgen treatment and this was suppressed by flutamide treatment. HPE cells derived from biopsies 1990-1 and 2CaP-1 did not respond to androgen treatment, but CAT reporter gene activity was still suppressed by flutamide, suggesting that these cells were still responsive to antiandrogen treatment. The HPE bioassay from biopsy 3771-1 was the most different in that androgens had little effect, but flutamide greatly stimulated reporter gene activity and the addition of androgen plus flutamide resulted in a synergist increase in CAT gene expression. Thus, these results suggest that protein profiles from the frozen sections correlate with the biological response of HPE cells originally derived from those of the same tissues. Furthermore, the protein profiles may be predictive of the response to androgen ablation therapy and could identify molecular markers which could be used to target therapies to specific tumor characteristics.

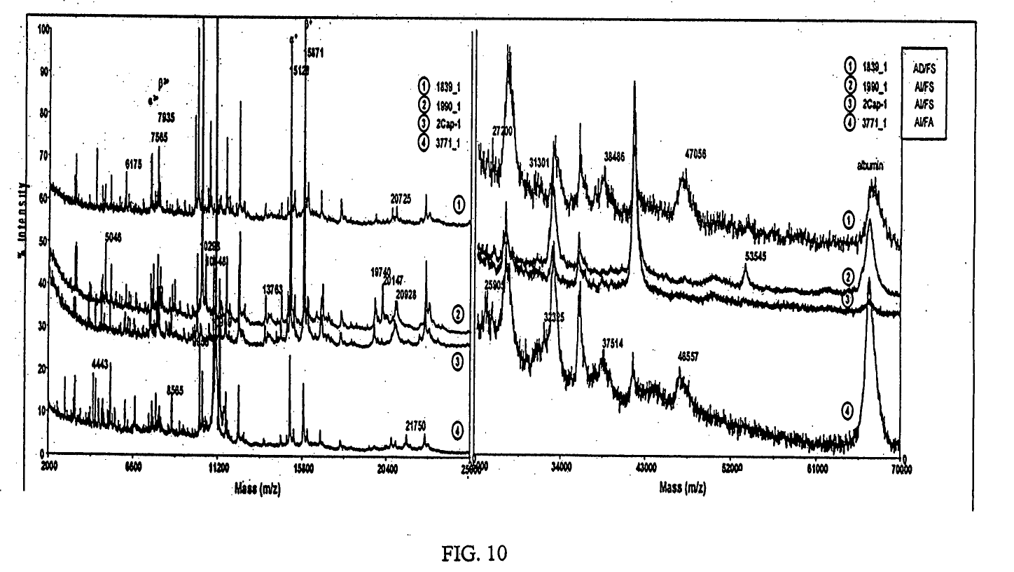

FIG. 11. Protein profile from a prostate needle biopsy. A 10 micron frozen tissue section from a prostate needle biopsy was cut and analyzed by MS as described above to demonstrate that there is sufficient tissue for MS analysis. This profile demonstrates that although the needles biopsies are small, protein profiles can still be obtained. The insert in the lower right-hand corner shows the biopsy section with two spots of matrix on it. Although not shown, the profile of the lower spot was nearly identical to that obtained in the upper spot, suggesting that the cancer in the needle biopsy was relatively homogenous throughout the tissue.

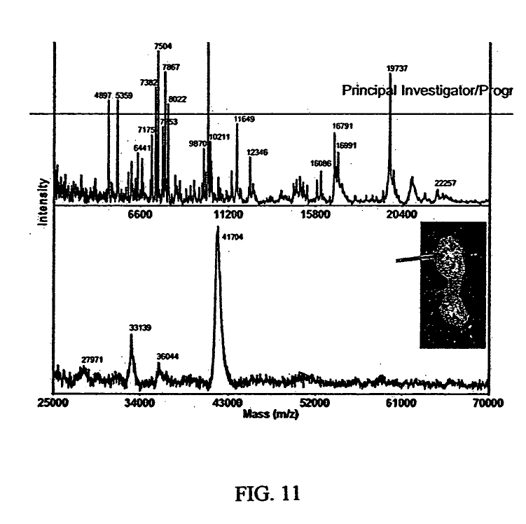

FIG. 12. Comparison of the needle biopsy profile with those obtained in the HPE bioassay. The upper panel represents the protein profiles for biopsies 1990-1 and 2CaP-1 (see FIG. 11). The HPE bioassay on prostate epithelial cells derived from these cells did not respond to androgen treatment, but flutamide suppressed reporter gene activity, suggesting that the tumor was still responsive to androgen ablation therapy. When compared to these protein profiles, the needle biopsy protein profile has the greatest similarity with biopsies 1990-1 and 2Cap-1, suggesting that if an HPE bioassay were performed, these cells would respond to hormonal treatment in a similar manner.

FIG. 13. Illustrates a comparison of the protein profiles along the length of the prostate biopsy. Protein profiles were obtained in 4 individual spots along the length of biopsy 1839-1 to detect any differences in protein expression in different regions of the section. The insert on the left shows the H&E stained histology section and the insert on the right is the frozen section spotted with matrix. The matrix spots have been numbered and correspond to the numbers on the protein profiles. Histological analysis indicated that the tumor tissue was consistent along the core and this was reflected in the mass spectra where few changes were observed in the sequential protein profiles. This analysis will allow us to identify regions of similar expression and discover regions where different profiles are generated. Thus, the tumor tissue should be able to be spatially localize from the stromal tissue and identify regions where expression of select proteins has changed. These select proteins may be markers for the progression from hormone responsive to hormone refractory prostate cancer cells.

### DESCRIPTION OF ILLUSTRATIVE EMBODIMENTS

Androgens are the primary growth factor for prostate cells and a commonly used approach to the management of advanced prostate cancer is the chemical or surgical removal of androgens. The adrenal glands produce up to 5% of serum androgens and therefore, antiandrogens such as flutamide or bicalutamide may be added to the treatment regime to suppress adrenal androgen production in what is referred to as maximum androgen blockade (MAB). Subsequently, regression is typically followed by monitoring the decline of serum PSA levels and the maintenance of nadir PSA levels. A subsequent rise of those levels is an indicator of prostate cancer progression. Although 85-90% of patients with advanced prostate cancer initially respond to this therapy, men with metastatic disease eventually develop androgen-independent prostate cancer (AIPC). To date, there is no biological assay for predicting when the disease progresses to androgen independence, no standard treatment for preventing the progression to hormone refractory disease and limited treatment for when the disease progresses to androgen independence.

In various embodiments of the invention, a bioassay may be used to analyze the activity of the androgen receptor AR in primary human prostate epithelial (HPE) cells derived from patient samples. In some embodiments, bioassay methods include administration of an androgen-regulatable expression construct. The androgen-regulatable expression construct includes an androgen-regulatable promoter element operatively linked to a reporter gene. In certain embodiments, a probasin ARR2PB promoter may be linked to a choramphenicol acetyl transferase (CAT) reporter gene, subcloned into the AdenoQuest™ AdBN adenoviral vector and used to generate adenoviral particles. HPE cells may be cultured out of patient samples, infected with the adenoviral particles and CAT gene activity can be measured in response to androgen (e.g., DHT or R1881) or antiandrogen (e.g., flutamide) treatment. Using embodiments of the assay described herein, the inventors have been able to measure the response of hormonal treatment or exposure at the transcriptional level. Typically, three primary subgroups (androgen profiles) of HPE cells have been identified: (1) those where androgen activated reporter gene expression and antiandrogen suppressed this activity, (2) those where androgen had little effect, but antiandrogen could still suppress reporter gene activity, and (3) those where antiandrogen acted as an agonist, enhancing reporter gene expression. The HPE bioassay demonstrates that prostate epithelial cells derived from patient biopsy samples respond to antiandrogen treatment in a similar manner as seen in patients undergoing androgen ablation therapy. Thus, the bioassay may be used to determine the sensitivity of a cancer to treatment regiment or to characterize a particular biological sample.

In certain embodiments, the biological data from HPE cells may be compared to protein profiles generated by mass spectrometry from frozen tissue sections (which were the original source of the HPE cells) to determine the correlation between the bioassay and the proteins expressed in the prostate tissue or biological sample. The protein profiles from the frozen sections typically correlate with the biological response of HPE cells originally derived from those of the same tissues and may be predictive of prostate cancer cells susceptible to androgen and antiandrogen treatment.

Biopsy tissue, for example a needle or punch biopsy, may also be profiled and compared with those from the frozen tissue punch biopsies. When compared to these protein profiles, the needle biopsy protein profile was typically identical to the subset of HPE cells where androgen had little effect, but antiandrogen could still suppress reporter gene activity. These results suggest that if an HPE bioassay were performed, these cells would respond to hormonal treatment in a similar manner. Thus, needle biopsy protein profiles may be predictive of the response to androgen ablation therapy and could be used to identify molecular markers that would be used to target therapies to specific tumors or tumor characteristics.

In certain embodiments of the invention, it is contemplated that sequential prostate needle biopsies may be obtained and analyzed (at least one pre-treatment and at least one when PSA levels begin to rise) from men undergoing routine treatment for advanced prostate cancer. HPE cells may be cultured directly from the needle biopsies for performing transfection assays (bioassays) to measure the biological activity of AR in response to androgen and antiandrogen treatment or exposure. The bioassay results will be compared to a second biopsy from the same patient after the disease has become hormone refractory. The tissues may also be profiled and the protein profiles correlated with the HPE bioassay.

Typically, there will be a correlation between the response to hormone treatment and the progression to androgen-independent disease. Furthermore, the protein profiles may provide a fingerprint of this progression and be predictive of the response to androgen and antiandrogen treatment in a given patient biopsy. The information generated by a bioassay, protein profiles or a combination thereof may be translated into markers for prognosis, provide an assay for predicting hormonal responsiveness to androgen deprivation, and provide targets for given prostate tumor characteristics.

Androgen-responsiveness, as determined by the methods described herein, may include one of the following profiles: (a) a first androgen-responsive profile, wherein cells demonstrate an enhanced transcription of a reporter gene operatively linked to an androgen-regulatable promoter upon exposure to androgens and demonstrate suppressed transcription of a reporter gene operatively linked to an androgen-regulatable promoter upon exposure to antiandrogens; (b) a second androgen-responsive profile, wherein cells do not demonstrate an enhanced transcription of a reporter gene operatively linked to an androgen-regulatable promoter upon exposure to androgens and demonstrate suppressed transcription of a reporter gene operatively linked to an androgen-regulatable promoter upon exposure to antiandrogens; (c) a first androgen-independent profile, wherein cells do not demonstrate an enhanced transcription of a reporter gene operatively linked to an androgen-regulatable promoter upon exposure to androgens and do not demonstrate enhanced transcription of a reporter gene operatively linked to an androgen-regulatable promoter upon exposure to antiandrogens; or (d) a second androgen-independent profile, wherein cells do not demonstrate an enhanced transcription of a reporter gene operatively linked to an androgen-regulatable promoter upon exposure to androgens and demonstrate enhanced transcription of a reporter gene operatively linked to an androgen-regulatable promoter upon exposure to antiandrogens.

I. Protein Profiling

Protein profiles have also been used in attempts to identify specific differences in the protein content of prostate tissue extracts and biological fluids from normal males and patients with prostate disease. The protein profiles from the frozen sections typically correlate with the biological response of HPE cells originally derived from those of the same tissues and may be predictive of prostate cancer cells susceptible to androgen and antiandrogen treatment.

A. MALDI-TOF-MS

Since its inception and commercial availability, the versatility of matrix assisted laser desorbtion ionization time-of-flight mass spectrometry (MALDI-TOF-MS) has been demonstrated convincingly by its extensive use for qualitative analysis. For example, MALDI-TOF-MS has been employed for the characterization of synthetic polymers (Marie et al., 2000; Wu et al., 1998), peptide and protein analysis (Zuluzec, et al., 1995; Roepstorff, 2000; Nguyen et al., 1995), DNA and oligonucleotide sequencing (Miketova et al., 1997; Faulstich et al, 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool—its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins.

Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

1. Biological Sample Preparation

In general, all reasonable efforts should be made to reduce excessive contamination in the samples. Always use the best quality solvents, reagents and samples. HPLC-grade solvents should be the standard in MALDI studies. Keep all samples in plastic containers. Glass containers can cause irreversible sample losses through adsorption on the walls, and release alkali metals into the analyte solution.

Optimum sample handling conditions for biological preparations usually involve non-volatile salts. Desalting might be necessary in the presence of excessive cationization, decreased resolution or signal suppression. Washing the analyte-doped matrix crystals with cold acidic water has been suggested as a very efficient way of desalting samples that have already been crystallized with the matrix. However, whenever possible, it is best to remove the salts, before the crystals are grown, using some of the techniques described later or other known techniques. There is a competition between protonation and cationization in MALDI when salts are present, and the choice between the two processes is still the subject of investigation.

When working with complex biological materials in MALDI it is often necessary to use detergents, otherwise the proteins, specially at less than mM concentrations, will be rapidly adsorbed on accessible surfaces. If no detergent is used; agglomeration and adsorption can effectively suppress protein peaks in the spectrum. The effect of detergents on MALDI spectra depends on the type of detergent and sample.

Nonionic detergents (TritonX-100, Triton X-114, N-octylglucoside and Tween 80) do not interfere significantly with sample preparation. In fact, it has even been reported that Triton X-100, in a concentration up to 1%, is compatible with MALDI and in some cases it can improve the quality of spectra. N-octylglucoside has been shown to enhance the MALDI-MS response of the larger peptides in digest mixtures. The addition of nonionic detergents is often a requirement for the analysis of hydrophobic proteins. Common detergents such as PEG and Triton, added during protein extraction from cells and tissues, desorb more efficiently than peptides and proteins and can effectively overwhelm the ion signals. Detergents often provide good internal calibration peaks in the low mass range of the mass spectrum.

Ionic detergents and particularly sodium dodecyl sulfate (SDS), can severely interfere with MALDI even at very low concentrations. Concentrations of SDS above 0.1% must be reduced by sample purification prior to crystallization with the matrix. The seriousness of this effect cannot be ignored given the wide application of MALDI to the analysis of proteins separated by SDS-PAGE. Polyacrylamide gel electrophoresis introduces sodium, potassium and SDS contamination to the sample, and it also reduces the recovered concentration of analyte. Once a protein has been coated with SDS, simply removing the excess SDS from the solution will not improve sample prep for MALDI: the SDS shell must also be removed. Typical purification schemes involve two phase extraction such as reversed-phase chromatography or liquid-liquid extraction.

Involatile solvents are often used in protein chemistry. Examples are: glycerol, polyethyleneglycol, β-mercaptoethanol, dimethyl sulfoxide (DMSO) and dimethylformamide (DMF). These solvents interfere with matrix crystallization and coat any crystals that do form with a difficult to remove solvent layer. If you must use these solvents and the dried-droplet method does not yield good results, try a different crystallization technique such as crushed-crystal method.

The use of buffers is often necessary in protein sample preparation to maintain biological activity and integrity. It is generally assumed that MALDI is tolerant of buffers. In cases where buffers are possible sources of interference, a trick that has been shown to work is to increase the matrix:analyte ratio. The effect of six common buffer systems, on the MALDI spectra of bovine insulin, cytochrome c and bovine albumin with DHB as a matrix has been studied (Wilkins et al., 1998).

In order to get “clean samples,” free of salts, buffers, detergents and involatile compounds, several experimental approaches have been tested with varying results. A number of researchers have attempted to establish “MALDI from synthetic membranes” as a general purification tool in protein biochemistry. In an extensive series of studies, analyte droplets were deposited on to polymeric membranes (porous polyethylene, polypropylene, analyte, nylon, Nafion, and others), washed in special solvents, and mixed with matrix to provide “clean” crystals. The approach is most useful for the direct analysis of proteins electroblotted from SDS-PAGE gels into synthetic membranes. In a more elaborate study, protein samples were desalted and freed of salts and detergents by constructing self-assembled monolayers of octadecylmercaptan (C18) on a gold coated MALDI probe surface. These surfaces were able to reversibly bind polypeptides through hydrophobic interactions allowing simultaneous concentration and desalting of the analyte.

Surface enhanced affinity capture (SEAC) was created (Hutchens et al., 1993) to facilitate desorption of specific macromolecules affinity-captured directly from unfractionated biological fluids and extracts, and can also be used as a means for sample purification. Direct analysis of affinity-bound analytes by MALDI TOF is now performed routinely and it is even possible to get customized affinity-capture sample probes from commercial sources.

Purification of analyte samples by traditional methods, such as alcohol or acetone precipitation, HPLC, ultrafiltration, liquid-liquid extraction, dialysis and ion exchange are always recommended; however, the effects of increased sample preparation time and sample recovery yields must be weighed carefully. It is possible to purify samples prior to analysis by using small, commercially available (or even home-made) C18 reverse-phase microcolumns or centrifugal ultrafiltration devices; however, such devices can still suffer from the same drawbacks as large-scale separation schemes. Note that acetone precipitation and dialysis usually do not remove enough detergent for MALDI sample preparation.

The degradation of signal intensity and resolution that results from excessive contamination can sometimes be eliminated by more extensive dilution of the protein in the matrix solution, a common trick is to try a 1:5 dilution series of the sample. Diluting the protein solution very often improves the MALDI signal, perhaps by diluting the contaminants while the matrix concentrates the analyte. This trick works well for hydrophobic proteins where the presence of lipids is suspected.

2. Matrix

Solubility in commonly used protein solvent mixtures is one of the conditions a “good” matrix must meet. Incorporating the protein or peptide (target or standard) into a growing matrix crystal implies that the protein and the matrix must be simultaneously in solution. Therefore, a matrix should dissolve and grow protein-doped crystals in commonly used protein-solvent systems. This condition should be expanded to any solvent system in which the analyte of interest will co-dissolve with the matrix. In practical terms, this means that the matrix must be sufficiently soluble to make 1-100 mM solutions in solvent systems consisting of: acidified water, water-acetonitrile mixtures, water-alcohol mixtures, 70% formic acid, etc.

The light absorption spectrum of the matrix crystals must overlap the frequency of the laser pulse being used. The laser pulse energy must be deposited in the matrix. Unfortunately the absorption coefficients of solid systems are not easily measured and are usually red shifted (Stokes shift) relative to the values in solution. The extent of the shifts varies from compound to compound. The solution absorption coefficients are often used as a guide, and typical ranges for commonly used matrix materials, at the wavelengths they are applied, are e=3000-16000 (lmol-1 cm-1). Uv-MALDI, with compact and inexpensive nitrogen lasers operating at 337 nm is the most common instrumental option for the routine analysis of peptides and proteins. IR-MALDI of peptides has been demonstrated but is not used in analytical applications. For UV-MALDI, compounds such as some trans-cinnamic acid derivatives and 2,5-dihydroxy benzoic acid have proven to give the best results.

The intrinsic reactivity of the matrix material with the analyte must also be considered. Matrices that covalently modify proteins (or any other analyte) cannot be applied. Oxidizing agents that can react with disulfide bonds and cysteine groups and methionine groups are immediately ruled out. Aldehydes cannot be used because of their reactivity with amino groups.

The matrix material must demonstrate adequate photostability in the presence of the laser pulse illumination. Some matrices become unstable, and react with the peptides, after laser illumination. Nicotinic acid, for example, easily looses —COOH when photochemically excited leaving a very reactive pyridyl group which results in several pyridyl adduct peaks in the spectrum. This is one of the reasons that the use of nicotinic acid has been replaced by more stable matrices such as SA and CHCA.

The volatility of the matrix material must be contemplated as well. From an instrumental perspective, the matrix crystals must remain in vacuum for extended periods of time without subliming away. Cinnamic acid derivatives perform a lot better in that respect when compared to nicotinic and vanillic acids.

The matrix must have a special affinity for analytes that allows them to be incorporated into the matrix crystals during the drying process. This is undoubtfully the hardest property to quantify and impossible to predict. In the current view of MALDI sample preparation, ion production in the solid-state source depends on the generation of a suitable composite material, consisting of the analyte and the matrix. As the solvent evaporates, the analyte molecules are effectively and selectively extracted from the mother liquor and co-crystallyzed with the matrix molecules. Impurities and other necessary solution additives are naturally excluded from the process.

The matrix molecules must possess the appropriate chemical properties so that analyte molecules can be ionized. Most of the energy from the laser is absorbed by the matrix and results in a rapid expansion from the solid to the gas phase. Ionization of the analyte is believed to occur in the high pressure region just above the irradiated surface and may involve ion-molecule reactions or reaction of excited state species with analyte molecules. Most commonly used matrix materials are organic acids and protonation, the addition of a proton to the analyte molecule to form (M+H)+ions, is the most common ionization mechanism in MALDI of peptides and proteins. Excited state proton transfer is a plausible mechanism for the charge transfer events that occur in the plume. Compounds, which perform a proton transfer under UV irradiation, are generally usable as matrices for UV-MALDI-MS. Whether the described proton transfer and the resulting metastable excited-state is involved in the ionization process or if it just offers an absorption band in the used wavelength area is not clear.

The final and definitive test for any potential matrix compound is to introduce the material in a laser desorption mass spectrometer and do a MALDI study. Many compounds form protein-doped structures that produce protein ions, but they are disqualified by other factors. The qualities that separate most matrix candidates from the ones that actually work are still very obscure and more studies are needed to improve the understanding of the effects involved.

Once a matrix compound has been proved to deliver ions in a MALDI source, it is also important to look at the performance of the material as far as the extent of matrix adduction to the analyte ions. Matrix adduct ions, (M+matrix+H)+, are usually observed in MALDI spectra; however, extensive adduct formation affects the ability to determine accurate molecular weights when the adductions are not well resolved from the parent peak. The best matrices have low intensity photo chemical adduct peaks.

MALDI is a soft ionization method capable of ionizing very large bioplymers while producing little or no fragmentation. The extent of fragmentation during desorption/ionization must be considered critically during matrix selection. Excessive fragmentation can cause decreased resolution. It is well known that the extent of fragmentation for proteins is strongly related to the matrix compound used. Some matrices are “hotter” than others, leading to more in-source (i.e., prompt) and post-source decay. A good example of a “hot” matrix material is CHCA which produces intense multiply charged ions in the positive ion spectra of proteins and contributes to significant fragmentation in the mass spectrometer.

Even after a matrix has been proved to be useful for a specific peptide or protein there is no algorithm other than trial-and-error to predict its applicability to other sample molecules. More than one matrix material is often required to get a complete representation of a complex mixture.

With a few exceptions, the development of new matrices has relied completely on commercially available compounds. It has been argued that this has limited the ability to effectively correlate matrix structure to MALDI function. More recent efforts (Brown et al., 1997), have tried to overcome this limitation through the intelligent synthesis of compounds that will provide a wide range of functionality. Most fine chemical manufacturers are aware of the utility of some of their compounds as MALDI matrices and have dedicated catalog numbers to those chemicals purified specifically for MALDI application. Matrix compounds are typically used as received from the manufacturer without any prior purification, and it is always a good idea to store them in the dark.

Most MALDI practitioners use MALDI for pure analytical purposes and are not interested in the discovery of novel MALDI materials. Luckily for them, there are a few compounds that provide consistently good results and can be relied upon for the routine analysis of peptides and proteins. Some of the most commonly used matrices are a-cyano-4-hydroxycinnamic acid (CHCA), gentisic acid, or 2,5-dihydroxy benzoic acid (DHB), trans-3-indoleacrylic acid (IAA), 3-hydroxypicolinic acid (HPA), 2,4,6-trihydroxyacetophenone (THAP), dithranol (DIT). The definitive choice of matrix material depends on the type of analyte, its molecular weight and the nature of the sample (pure compound, mixture or raw biological extract). In all cases the performance of the matrix material is influenced by the choice of solvent. Experimentation (i.e., trial-and-error laced with a few educated guesses) is generally the only way to find the best sample preparation conditions. Some examples of compounds that have also been used for MALDI of peptides and proteins include: hydroxy-benzophenones, mercaptobenthothiazoles, β-carbolines and even high explosives.

Most matrices reported to date are acidic, but basic matrices such as 2-amino-4-methyl-5-nitropyridine and neutral matrices such as 6-aza-2-thiothymine (ATT) are also used, which extends the utility of MALDI to acid sensitive compounds.

Matrix peaks are often used for low mass calibration in the mass axis calibration procedure. [M⁺Na]⁺ and [M⁺K]⁺ peaks are also observed if samples are not carefully desalted.

a. Matrix Suppression

At appropriate matrix to analyte mixing ratios, small to moderately sized analyte ions (1000-20000 Da) can fully suppress positively charged matrix ions in MALDI mass spectra. This is true for all matrix species, and is observed regardless of the preferred analyte ion form (protonated or cationized). Since the effect has been observed with a number of matrices including CHCA and DHB, it seems to be a general phenomenon in MALDI. Along with the fact that fragmentation is weak in MALDI, this leads to nearly ideal mass spectra with a strong peak for the analyte ions and no other signals present.

b. Co-Matrices (Matrix Additives)

Several additives have been added to MALDI samples to enhance the quality of the mass spectra. Additives, also known as co-matrices, can serve several different purposes: (1) increase the homogeneity of the matrix/analyte deposit, (2) decrease/increase the amount of fragmentation, (3) decrease the levels of cationization, (4) increase ion yields, (5) increase precision of quantitation, (6) increase sample-to-sample reproducibility, and (7) increase resolution.

The use of co-matrices is much more widespread in the analysis of oligonucleotides, where ammonium salts and organic bases are very common additives. Some MALDI researchers believe that the use of additives may provide the most general and simplest means of improving the current matrix systems. Continuing efforts are needed to evaluate the effects of co-matrices on the MALDI process, and to further characterize additives for such purposes. Some examples of additives used in peptide and protein measurements are: common matrices, bumetanide, glutathione, 4-nitroaniline, vanillin, nitrocellulose and L(−) fucose.

The addition of ammonium salts to the matrix/analyte solution substantially enhances the signal for phosphopeptides. This has been used to allow the identification of phosphopeptides from unfractionated proteolytic digests. The approach works well with CHCA and DHB and with ammonium salts such as diammonium citrate and ammonium acetate.

3. Solvent Selection

Solvent choice remains to this day a trial-and-error process that is governed by the need to maintain analyte solubility and promote the partitioning of the analyte into the matrix crystals during drying of the analyte/matrix solution. As a general rule, it is best to first find the appropriate solvent for the sample.

Once the analyte has been completely dissolved, a solvent should be chosen for the matrix that is miscible with the analyte solvent. In some cases, such as the analysis of peptides and proteins, or oligonucleotides, the appropriate solvents are well known. In the analysis of peptides/proteins 0.1% TFA is the solvent of choice, and for oligonucleotides, pure 18 Ohm water. The matrices for these analytes are dissolved in ACN/0.1% TFA and ACN/H₂O, respectively. What follows is a more detailed look at the rules governing the choice of solvents for analyte and matrices in MALDI.

Solubility of the analyte in the solvent system is one of the most important parameters to be considered during solvent selection. The analyte must be truly dissolved in the solvent at all times. Making a slurry of analyte powder and solvent never leads to good results.

Two solvent systems are usually involved in a MALDI sample preparation procedure. There is a solvent system for the analyte sample, and a different solvent for the matrix. In most sample preparation recipes (dried-droplet technique), an aliquot of the matrix solution is mixed with an aliquot of the protein solution to make a crystal-forming mother liquor. Both matrix and analyte solvents must be chosen carefully. It is important that neither the matrix nor the analyte precipitate when the two solutions mix. Particular care must be taken when the analyte's solvent does not contain any organic solvent, which may lead to precipitation of the matrix during mixing. Attention must also be paid to inadvertent changes in solvent composition as caused by selective evaporation of organic solvents from aqueous solutions. Tubes of analyte and matrix solutions should be kept closed while not in use to avoid evaporation.

Analyte solubilization is the key to the successful analysis of hydrophobic proteins and peptides. Owing to their limited solubility in aqueous solvents, alternative solvents for both the matrix and the analyte have been carefully investigated. Several solubilization schemes have been successfully applied including strong organic acids (i.e., formic acid), detergent solutions and non-polar organic solvents. Non-ionic detergents, that improve the solubility of peptides and proteins, are often added to sample solutions to improve the quality of spectra. The effect has been reported in the literature for the characterization of high molecular weight proteins in very dilute solutions. Use of detergents for cell profiling has extended the detectable mass range to about 75 kDa.

The surface tension of the solvent system must also be considered during the selection process. At low surface tension the matrix-analyte droplets spread over a large surface area resulting in a dilution effect and lowering the ion yields. In general, water-rich solvents exhibit adequate surface tension and allow the formation of reproducible round-shaped deposits with high crystal density. Low surface tension solvents, such as alcohols and acetone, provide wide spread and irregularly shaped crystal beds. Careful adjustment of the solvent surface tension is needed for MALDI targets with closely spaced sample wells and for sample preparation procedures relying on robotic sample loading.

The volatility of the solvent must also be considered. Fast solvent evaporation results in smaller crystals with more homogeneous analyte distributions. However, rapid crystallization also shows increased cationization, favors low molecular weight components in mixtures and provides very thin crystal beds that can only handle a few laser shots per spot. Volatile solvents require more skill from the operator since they must be handled quickly to avoid premature precipitation of the matrix in the pipette tips as caused by excessive solvent evaporation. Fast evaporating solvents such as acetone and methanol have reduced surface tension and form very wide and irregularly shaped MALDI deposits. The use of volatile solvents to obtain microcrystals during sample preparation can often be substituted with the “acetone redeposition” technique. In this technique, the dried MALDI sample (prepared with non-volatile solvents) is dissolved in a single drop of acetone and, as the acetone evaporates, the sample crystallizes to form a more homogeneous film.

Involatile solvents commonly used in protein chemistry must be avoided. Examples are glycerol, polyethyleneglycol, β-mercaptoethanol, dimethylsulfoxide, and dimethylformamide. These solvents interfere with matrix crystallization and coat any crystals that do form with a difficult to remove solvent layer. The crushed crystal method was specifically developed to deal with their presence.

The pH of the evaporating solvent system must be less than 4. Most of the MALDI matrix materials used for peptides and proteins are organic acids that become ions at pH>4, completely changing their crystallization properties. Solvent acidity affects the protein binding to matrix crystals and it can even modify the conformation of the proteins. Analyte conformation has been shown to influence MALDI Ion yields. The addition of trifluoroacetic acid (TFA) and formic acid (FA) to matrix solutions is common practice to assure the correct acidity during evaporation of the analyte-matrix droplet. Another common trick is to use 0.1% and 1% TFA, instead of pure water, as protein sample solvents. The acidity of the solution must be carefully optimized in MALDI of mixtures to assure no components are being excluded from the crystals.

The reactivity of the solvent system with the analyte must be contemplated. A common problem of using strongly acidic solvents is cleavage of acid-labile peptide bonds, such as aspartic acid's proline bond. Cleavage of this bond in small and large proteins has been observed after sample preparation and cleavage products increase in intensity with time.

A potential problem with using formic acid as a solvent, or solvent component, is its reactivity toward serine and threonine residues in proteins. Formyl esterification of those amino acids results in the production of satellite peaks at 28 Da intervals of higher molecular weight. As a result, exposure to formic acid should be avoided in any studies using exact mass measurements. If the procedure must use formic acid, exposure should be kept as short as possible. Formic acid, 70%, is the best solvent for CNBr peptide cleavage. Dilute HCl (0.1 N) may also be used; however, care must be taken to neutralize the solution's pH before evaporating the solvent to dryness. A protocol has been reported for deformylation of formylated peptides generated during CNBr cleavage by treatment with ethanolamine (Tan et al., 1983). Concentrated TFA is also known to react with free amino acids.

The composition of the solvent is an important parameter that can influence the outcome of a MALDI study. The selection of solvent components is affected by the analyte type and its molecular weight and by the matrix material being used. The solvent system must be capable of dissolving the matrix and the analyte at the same time. It must also allow for the selective inclusion of the analyte into the matrix crystals during the drying process.

Hydrophilic peptides and protein samples are usually dissolved in 0.1% TFA. Matrices are often dissolved, at higher concentrations, in solvent systems consisting of up to three components. Common matrix solvent components are acetonitrile (CH₃CN), small alcohols (methanol, ethanol 2-propanol), formic acid, dilute TFA (0.1-1% v/v) and pure water. TFA seems to yield spectra with higher mass resolution than formic acid; however, and particularly for mixtures, it is always advisable to try a range of solvents.

Oligonucleotides are mostly dissolved in pure water. Although, it is advised in all cases to use HPLC-graded solvents, deionized H₂O is recommended in the case of oligonucleotides. This is due to the fact that HPLC-grade water is acidic and can contain variable concentration of salts. The solvent most commonly used for HPA and THAP (oligonucleotide matrices) is a 1:1 v/v of ACN/H₂O. The additive that is used with these matrix solutions, ammonium bicitrate, is either dissolved in H₂O and later mixed with the matrix solutions or the matrices are dissolved in a solution of ammonium bicitrate in ACN/H₂O.

In the analysis of organic molecules or polymers, it is important to first find the optimum solvent for the sample and from there, depending on what the appropriate matrix for that compound is, the matrix can be dissolved in the same solvent as the sample or in a solvent that is miscible with the analyte solution.

Hydrophobic peptides (not soluble in water) are dissolved in water-free systems such as chloroform/alcohol or formic acid/alcohol mixtures and the matrix is usually dissolved in the same or very similar solvent. A nonionic detergent is often added to improve solubility and ion yields.

Solvent proportions in a solvent mixture can affect the ion yields in a MALDI study. A complete sample preparation protocol should include optimization of the relative concentrations of solvents in a mixture. For example, it has been demonstrated that small variations in the water content of alcohol-water mixtures can significantly affect ion yields. Very often the choice of concentrations can be as critical as the choice of components.

The variety of choices and effects that MALDI users must consider during solvent optimization must not be considered as a drawback for the MALDI technique. It is in fact, the ability to operate with a wide range of solvents and in the presence of impurities that has allowed MALDI to be used for the mass spectrometric characterization of all kinds of biological and synthetic polymers.

4. Substrate Selection

When designing effective MALDI sample preparation methods for analysis, attention must be given to the interaction of analytes with the substrate.

Most MALDI samples are prepared on and desorbed/ionized from multi-well metallic sample-plates made out of vacuum compatible stainless steel or aluminum. The role of the metal substrate in the desorption/ionization process is not well understood, but the surface conductivity of the metal is often considered essential to preserve the integrity of the electrostatic field around the sample during ion ejection. The hard metals can be machined and formed to high precision, and can also be easily cleaned and polished to provide the smooth surfaces needed for high resolution and high mass accuracy. The analyte/matrix crystals strongly adhere to metal surfaces providing very rugged samples that can be stored for long periods of time and washed for purification purposes.

Both stainless steel and aluminum are chemically inert to the matrix systems used and do not contribute metal ions to the cationization of the analyte during ion formation. Copper as a substrate, on the other hand, has been demonstrated to form adducts with both matrix and analyte during desorption (Russell et al., 1999). The effect is particularly dramatic with the matrix CHCA and leads to several peaks at molecular weights above the protonated ions. The extra peaks are generally viewed as a problem for the analysis of proteins, particularly when they are not clearly resolved from the protonated ion signal. However, Cu adduction can be exploited in MALDI post-source decay studies because [M⁺Cu]⁺ ions fragment in ways different from the protonated ones, providing valuable extra sequencing information.

Most MALDI sources use a solid sample plate and irradiation is done from the front (reflection geometry); however, use of transmission geometry to desorb the analyte/matrix samples is possible. In the transmission geometry the laser irradiation and the mass spectrometer's analyzer are on opposite sides of the thin sample. The substrates used in the two case studies were quartz and plastic-coated grids (Formvar on zinc or copper).

Plastic is the second most common material used in MALDI sources as a substrate. Significant attention must be given to the interaction of the peptides and proteins with the polymeric surface. (Kinsel et al., 1999) The influence of polymer surface-protein binding affinity on protein ion signals has been studied, and it showed that as the surface-protein binding affinity increases the efficiency of MALDI of the protein decreases.

Desorption of high mass proteins (>100 kDa), directly deposited on polyethylene membranes was demonstrated (Blackledge et al., 1995) and the spectra obtained were identical or better than with standard metal substrates. Similar improvements were observed by Guo (1999) while desorbing DNA and proteins directly from Teflon-coated MALDI probes. The use of a Nafion substrate with certain matrices can significantly enhance the signals obtained over those observed with a stainless-steel probe. Its use has been demonstrated to be particularly effective in analyzing real biological mixtures without pre-purification and used with polypropylene, polystyrene, teflon, nylon, glass and ceramics as matrix crystal supports with no noticeable decrease in performance relative to all-metal constructions (Hutchens et al., 1993).

The use of plastic membranes as sample supports has recently been adopted as a means of both sample purification and sample delivery into the mass spectrometer. If the analyte can be selectively adsorbed (hydrophobic interactions) onto the membrane, interfering substances can be washed off while the analyte is retained. Purification by on-probe washing results in lower sample loss than pre-purification by traditional methods. Polyethylene and polypropylene surfaces have been used to conduct on-probe sample purification. (Woods et al., 1998) Similarly, poly(vinylidene fluoride) based membranes have been used to extract and purify proteins from bulk cell extracts and for the removal of detergents, and a method has been developed for probe surface derivatization to construct monolayers of C18 on MALDI Probes. (Orlando et al., 1997) Non-porous polyurethane membrane has been used as the collection device and transportation medium of blood sample analysis, followed by direct desorption from the same membrane substrate in a MALDI-TOF spectrometer (Perreault et al., 1998). Sample purification and proteolytic digest right on the probe tip, with minimal sample loss, was also possible with this substrate. Nitrocellulose, used as a sample additive or as a pre-deposited substrate, has been used by several researchers to improve MALDI spectra quality, to induce matrix signal suppression, and to rapidly detect and identify large proteins from Escherichia coli whole cell lysates in the mass range from 25-500 kDa.

Direct analysis of SDS-PAGE-separated proteins electroblotted onto membranes using MALDI-MS has been performed by a large number of MALDI users. In all cases, the membrane with the blotted protein spot is attached to the probe tip for direct MALDI analysis. The matrix is added to the protein spots by soaking the membrane with matrix solution. The incorporation of the proteins and peptides into the matrix crystals relies on the ability of the matrix solution to solvate the proteins adsorbed on the membrane. UV as well as IR irradiation are used to desorb/ionize the analyte molecules, with IR offering the advantage of larger penetration-depth into the membrane. Peptides produced after enzymatic or chemical digestion of proteins blotted onto a membrane have also been analyzed by MALDI, providing one of the fastest paths for protein identification after 2-D Gel separation. Poly(vinylidenefluoride) (PVDF) based membranes have been most commonly evaluated and used for these purposes. Other membranes, such as Nylon, Zitex, and polyethylene have also been found to be useful for the detection of dot blotted proteins by MALDI MS. A study demonstrates the capabilities of IR-MALDI can analyze electroblotted proteins directly from PVDF membranes, compare different membrane materials, and looks into on-membrane digestions and peptide mapping (Schleuder et al., 1999). The link between gel electrophoresis and MALDI MS has been taken one step further by introducing dried matrix-soaked gels into their mass spectrometers for direct MALDI analysis of the intact, and in-gel-digested, proteins (Philip et al., 1997). The method provides masses of both intact and cleavage products without the time and sample losses associated to electroelution or electroblotting. The key to their success is the use of ultrathin polyacrylamide gels, which dry to a thickness of 10 mm or less and which have the additional advantages of rapid preparation and electrophoresis run times. The methods are applied to isoelectric focusing (IEF), native and SDS-PAGE gels. When used in combination with IEF gels, this option makes it possible to run “virtual 2-D gels” in which proteins are resolved in the first dimension on the basis of their charge, whereas the second dimension is MALDI-MS-measured molecular weight instead of SDS-PAGE. The effects of the substrate on the MALDI signal must be carefully considered and accounted for in these studies. Mass accuracy in desorption from gels is an important concern. Several effects conspire against high mass accuracy determinations: (a) uneven gel thicknesses, (b) difficulty mounting gels flat and (c) surface charging of the dielectric material are the three most serious problems. Delayed extraction overcomes some of the mass accuracy limitations, and accuracy to better than 0.1% is readily obtained.

Another recent development in the MALDI field is the use of molecularly tailored MALDI-probe-substrates chemically modified to selectively capture specific analytes from solution prior to mass spectrometry (Hutchens et al., 1993). The efficacy of affinity capture techniques has been demonstrated (originally termed surface enhanced affinity capture (SEAC) mass spectrometry). In the published example of SEAC, agarose beads with attached single strand DNA were used to capture lactoferrin from pre-term infanturine. After these beads were incubated in the urine sample, the beads were removed, washed, placed directly on the MALDI probe tip and analyzed with conventional MALDI. The capture agent used as a substrate did not seem to degrade the performance of the MALDI-MS. Since this original report, on-probe immunoaffinity extraction has become common place in many laboratories, and there is even commercial sources that can supply affinity-capture probes tailored to specific analysis requirements.

Rapid peptide mapping has been accomplished using an approach in which the analyte is applied directly to a mass spectrometric probe tip that actively performs the enzymatic degradation, i.e., the probe substrate carries the enzymatic reagent. Applying the analyte directly to the probe tip increases the overall sensitivity of peptide mapping analysis. High on-probe enzyme concentrations provide digestion times in the order of a few minutes, without the adverse effect of autolysis peaks. Bioreactive probe tips have been used routinely for the proteolytic mapping and partial sequence determination of picomole quantities of peptide.

5. Crystallization Methods

With minor modifications, the original and simple sample preparation procedure introduced by Hillenkamp and Karas (1988) has remained intact for over a decade, and it is commonly referred to as the dried-droplet method: An aqueous solution of the matrix compound is mixed with analyte solution. A 1 mL droplet of this solution is then dried resulting in a solid deposit of analyte-doped matrix crystal that is introduced into the mass spectrometer for analysis.

The trick is to find matrix molecules that will dry out of solution with analyte molecules in the resulting matrix crystals and that will enable the MALDI process. Poor sample preparation will yield low resolution, poor reproducibility and degraded sensitivity. MALDI optimization is primarily an empirical process that involves a significant amount of trial-and-error. Every choice during sample preparation can potentially affect the outcome of the MALDI measurement. It is not unusual to test a few different approaches before choosing the optimum protocol for sample preparation. The following are a variety of methods used for crystallization.

a. Dried Droplet

The dried-droplet method is the oldest and has remained the preferred sample preparation method in the MALDI community.

The analyte/matrix crystals may be washed to etch away the involatile components of the original solution that tend to accumulate on the surface layer of the crystals (segregation). The procedure most often recommended is to thoroughly dry the sample (dessicator or vacuum dry) followed by a brief immersion in cold water (10 to 30 seconds in 4° C. water). The excess water is removed immediately after, by flicking the sample stage or by suction with a pipette tip.

This method is surprisingly simple and provides good results for many different types of samples. Dried droplets are very stable and can be kept in vacuum or refrigerator for days before running a MALDI study.

The dried-droplet method tolerates the presence of salts and buffers very well, but this tolerance has its limits. Washing the sample as described above can help; however, if signal suppression is suspected, a different approach should be tried (see crushed-crystal).

The dried-droplet method is usually a good choice for samples containing more than one protein or peptide component. The thorough mixing of the matrix and analyte prior to crystallization usually assures the best possible reproducibility of results for mixtures.

A common problem in the dried droplet method is the aggregation of higher amounts of analyte/matrix crystals in a ring around the edge of the drop. Normally these crystals are inhomogeneous and irregularly distributed, which is the reason MALDI users often end up searching for “sweet spots” on their sample surfaces. As an example, it has been observed that peptides and proteins tend to associate with the big crystals of 2,5-dihydroxybenzoicacid that form at the periphery of air dried drops containing aqueous solvent, whereas the salts are predominantly found in the smaller crystals formed in the center of the sample spot at the end of crystallization. In a clever set of studies, Li et al. (1996) used confocal fluorescence to demonstrate that with the dried-droplet method, the analyte is not uniformly distributed among or within the matrix crystals. In fact, some crystals show no analyte at all.

Most well-written MALDI software packages allow for automated sweet-spot searching during data acquisition, a procedure by which the sample surface is scanned with the laser beam until a portion yielding strong signals is located.

Another problem that is often observed during crystallization is what is known as segregation: as the solvent evaporates and the matrix crystallizes, the salts and some of the analyte are excluded from matrix crystals. This is particularly important in cases where cationization is the ionization mechanism, such as in the case of synthetic polymers and carbohydrates. Component segregation yields an inhomogeneous mixture of analyte throughout the sample, resulting in highly variable analyte ion production as the laser is moved across the sample surface.

b. Vacuum Drying

The vacuum-drying crystallization method is a variation of the dried-droplet method in which the final analyte/matrix drop applied to the sample stage is rapidly dried in a vacuum chamber. Vacuum-drying is one of the simplest options available to reduce the size of the analyte/matrix crystals and increase crystal homogeneity by reducing the segregation effect. It is not a widespread sample preparation method, because of its mixed results and extra hardware requirements.

When it works, vacuum-drying provides uniform crystalline deposits with small crystals. It greatly improves spot-to-spot reproducibility and minimizes the need to search for “sweet spots”. The formation of smaller crystals offers the added advantage of thinner samples and improved mass accuracy and resolution. Reductions in the amount of laser power required for ion formation have been reported for vacuum dried samples compared to similarly prepared air or heat dried samples.

The main disadvantages of vacuum-drying are that it is not guaranteed to work better than dried droplet in all cases, and it requires accessory vacuum hardware that many analytical laboratories might not have available. Peptides and proteins analyzed with the vacuum-drying method tend to exhibit extensive alkali cation adduction. This can be substantially reduced by washing the crystals directly on the probe with cold water. With evaporation times beyond 20 seconds in a vacuum system, the vacuum drying effects becomes less pronounced.

C. Crushed Crystal

The crushed-crystal method was specifically developed to allow for the growth of analyte doped matrix crystals in the presence of high concentrations of involatile solvents (i.e., glycerol, 6M urea, DMSO, etc.) without any purification.

The dried-droplet method is widely used because it is simple and effective. Good signals are obtained from initial solutions that contain relatively high concentrations of contaminants (salts and buffers). Many real analytical samples contain those materials and the capacity to tolerate these impurities has an enormous practical importance. However, there are limits to the contamination tolerance of the dried-droplet method. Particularly, the presence of significant concentrations of involatile solvents reduces, or totally eliminates, the ion signals. Examples of the most common of these solvents are dimethyl sulfoxide, glycerol and urea. Removal of the involatile solvents may not be possible if they are needed to dissolve or stabilize the analyte.

The dried-droplet method forms crystals randomly throughout the droplet as the solvent evaporates. The surface of the droplet is the preferred site for initial crystal formation. The crystals form at the liquid/air interface and are then carried into the bulk of the solution by convection. The final sample deposit is littered with those crystals, and if no involatile solvent is present they become adhered to the substrate. If involatile solvents are present, the crystals might either not form or remain coated with the solvent, preventing them from attaching to the substrate. Even if crystals are formed and the deposit is introduced into the mass spectrometer, a coating of involatile solvent usually suppresses the ion signals. Attempts to wash the crystals usually results in their loss, because they are not securely bonded to the substrate.

The crushed-crystal method is operationally similar to the dried-droplet method, but the results are very different, particularly in the presence of involatile solvents. In this method rapid crystallization directly on the metal surface is seeded by the nucleation sites provided by the smeared matrix bed that is crushed on the metal plate prior to sample application. Crystal nucleation shifts from the air/liquid interface to the surface of the substrate and microcrystals formed inside the solution where the concentrations change slower. The polycrystalline film adheres to the surface so the crystallization can be halted any time by washing off the droplet before its volume decreases significantly.

The films produced are also more uniform than dried-droplet deposits, with respect to ion production and spot-to-spot reproducibility.

The disadvantage of the crushed-crystal method is the increase in sample preparation time caused by the additional steps. It does not lend itself to automation for high throughput applications. It requires strict particulate control during solution preparation to eliminate the presence of undissolved matrix crystals that can shift the nucleation from the metal surface to the bulk of the droplet.

d. Fast Evaporation

The fast-evaporation method was introduced by Vorm et al. (1994) with the main goal of improving the resolution and mass accuracy of MALDI measurements. It is a simple sample preparation procedure in which matrix and sample handling are completely decoupled.

For crystal washing it is recommend to wash the crystals prior to their introduction into the TOF spectrometer. A large droplet of 5-10 mL of water or dilute aqueous organic acid (i.e., 0.1% TFA) is applied on top of the sample spot. The liquid is left on the sample for 2-10 seconds and is then shaken off or blown off with pressurized air. The procedure can be repeated once or twice. The washing liquid must be free of alkali metals and should be neutral or acidic (i.e., 0.1% TFA).

Pneumatic spraying: Pneumatic spraying of the matrix-only layer has been suggested as an alternative for fast evaporation. The process delivers stable and long lived matrix films that can be used to precoat MALDI targets.

The fast-evaporation method provides polycrystalline surfaces with roughnesses 10-100 times smaller than equivalent dried-droplet deposits. Confocal fluorescence studies demonstrated that, across an entire sample deposition area, the analyte is more uniformly distributed than with the dried-droplet method.

The improved homogeneity of the sample surface provides several advantages.(1) Faster data acquisition. All spots on the surface result in similar spectra under the same laser irradiance. No sweet-spot hunting and less averaging. The outcome of the first few laser shots is usually enough to decide the outcome of a study. (2) Better correlation between signal and analyte concentration (still not a quantitative technique). (3) More reproducible sample-to-sample results. (4) Improved sensitivity. The peptides have been detected down to the attomole level. The higher ion signals are explained as the result of the increased surface area of the smaller crystals combined with the preferential localization of the analyte molecules on the outer layers of the crystals from where the MALDI signal is believed to originate. (5) Improved washability. Salts and impurities are more easily washed off the sample deposits because the crystals are more securely bonded to the metal surface and to each other. (6) Improved resolution and mass measurement accuracy. Resolution improvements of at least a factor of two have been reported compared to dried-droplet results. The improved mass accuracy can often eliminate the need for internal standards. (7) Matrix surfaces can be prepared in advance. Precoated sample plates prepared by fast-evaporation of matrix solution on the sample spots are available from a few commercial sources.

Some of the disadvantages that have been associated with this method are as follows. (1) It does not provide reproducible sample-to-sample data for peptide and protein mixtures. If the protein or peptide sample contains more than one component, it is best to try the dried-droplet or overlayer method first. The thorough mixing of the analyte and matrix solutions prior to deposition increases the reproducibility of the spectra obtained. (2) Because the layer of protein-doped matrix on each crystal is usually very thin, it only produces ions for a few shots on a laser spot. The laser spot must constantly move to a fresh location to maintain the signal levels. This results in reduced duty cycle for the data acquisition loop, and reduced throughput. (3) Working with very volatile solvents such as acetone makes it difficult to make reproducible sample spots. The solvent has a small surface tension and it spreads uncontrollably along the metal surface. Some varying amount of solvent is always lost to evaporation before the matrix-only droplet is delivered. (4) The method is very effective for the analysis of peptides but is not as effective for proteins. The two-layer method should be tried first in the case of proteins.

e. Overlayer (Two-Layer, Seed Layer)

The overlayer method was developed on the basis of the crushed-crystal method and the fast-evaporation method. It involves the use of fast solvent evaporation to form the first layer of small crystals, followed by deposition of a mixture of matrix and analyte solution on top of the crystal layer (as in the sample matrix deposition step of the crushed-crystal method). The origin of this method, and its multiple names, can be traced back to the efforts of several research groups (Li et. al., 1999).

The difference between the fast evaporation and the overlayer method is in the second-layer solution. The addition of matrix to the second step is believed to provide improved results, particularly for proteins and mixtures of peptides and proteins.

The overlayer method has several convenient features that make it a very popular approach. (1) It naturally inherits all the advantages detailed in the fast evaporation method, and it avoids some of its limitations. (2) It provides enhanced sensitivity and excellent spot-to-spot reproducibility for proteins beyond what is possible with the fast-evaporation method. This enhancement is likely due to improved matrix isolation of the analyte molecules on the crystal surfaces in the presence of the surplus of matrix molecules. (3) With the careful optimization of the second-layer analyte/matrix solution, the overlayer method is found to be very effective for the analysis of complicated mixtures containing both peptides and proteins. The ability to manipulate the second layer conditions adds flexibility to the sample preparation.

f. Sandwich

The sandwich method is derived from the fast-evaporation method and the overlayer method. It was reported for the first time by Li (1996), and used for the analysis of single mammalian cell lysates by mass spectrometry. The report also included the description of a Microspot MALDI sample preparation to reduce the sample presentation surface to a minimum.

In the sandwich method the sample analyte is not premixed with matrix. A sample droplet is applied on top of a fast-evaporated matrix-only bed as in the fast-evaporation method, followed by the deposition of a second layer of matrix in a traditional (non-volatile) solvent. The sample is basically sandwiched between the two matrix layers.

g. Spin Coating

The preparation of near homogeneous samples of large biomolecules, based on the method of spin-coating sample substrates was reported for the first time by Perera (1995). In the original report, samples were deposited on 1″ diameter stainless steel and quartz plates, and large volumes (3-10 mL) of the premixed sample solution were used. The spin coater was home-built and it operated at about 300 rpm, producing evenly spread crystal deposits in air. The samples were very homogeneous and generated highly reproducible and much enhanced molecular-ion yields from all regions of the sample target.

Spin coating the analyte/matrix samples works well and it usually delivers more homogeneous deposits on single-spot sample stages. However, it is not a viable option for MALDI plates with multiple sample wells of the kind found in all modern commercial instruments.

h. Slow Coating

It is possible to grow large, protein doped matrix crystals under near equilibrium conditions, rather than in a rapidly drying droplet (Beavis and Xiang, 1993). Supersaturated matrix solutions containing protein will form crystals that can be used directly in an ion source. Supersaturation can be achieved by heating, cooling or slow evaporation. The protein-doped crystals can be cleaved to expose well defined faces to the laser beam.

In general the slow crystallization approach favors the detection of high mass components over low mass peptides, regardless of pH and solution.

Producing large protein-doped crystals has several disadvantages compared to the fast drying (non-equilibrium) crystallization techniques described elsewhere: (1) It is slower. Crystals take hours to grow, definitely not practical for large-scale, high-throughput applications. (2) Peak broadening is often observed. (3) High mass accuracy is out of the question due to the irregular geometry of the sample bed. (4) Growing crystals requires more analyte (10-100×) than traditional methods.

However, even with those difficulties some advantages are also realized: (1) Crystals can be grown from solutions with involatile solvents at concentrations that suppress ion signals from dried droplet studies. (2) High concentrations of non-proteinaceous solutes do not affect crystal doping. Detergents are an exception. (3) Mixtures of polypeptides can be incorporated into crystals and analyzed. (4) Crystals can be easily manipulated. Common operations are washing, cleaving, etching and mounting. (5) The crystals are very rugged. (6) The crystals provide more defined starting conditions for fundamental MALDI ionization mechanism studies.

i. Electrospray

Electrospray as a sample deposition for MALDI-MS was suggested by Owens and Axelsson (1997). In this technique, a small amount of matrix-analyte mixture is electrosprayed from a HV-biased (3-5 KV) stainless steel or glass capillary onto a grounded metal sample plate, mounted 0.5-3 cm away from the tip of the capillary.

Electrospray sample deposition creates a homogenous layer of equally sized microcrystals and the guest molecules are evenly distributed in the sample. The method has been proposed to achieve fast-evaporation and to effectively minimize sample segregation effects. The presence of cation adducts in the MALDI spectra from electrodeposited samples demonstrates that solution components are less segregated than in equivalent dried-droplet deposits.

Electrospray matrix deposition was used (Caprioli et al., 1997) to coat tissue samples during the MALDI based molecular imaging of peptides and proteins in biological samples. Matrix-only solution was electrosprayed on TLC plates for the direct MALDI analysis of the impurity spots of tetracycline samples (Clench et al., 1999).

Electrospray deposited samples have been shown to give several advantages over traditional droplet methods: (1) The reproducibility of MALDI results from spot-to-spot within one sample deposit, and from sample-to-sample for multiple depositions, is much improved. Typical sample-to-sample variations are in the 10 to 20% range. (2) The correlation between analyte concentration and matrix signal is also improved. Quantitation with internal standards has been reported by Owens. (3) The sample deposits are much more resistant to laser irradiation. More shots can be collected from any single laser spot location. (4) The method offers a possible path for interfacing MALDI sample preparation to Capillary electrophoresis and liquid chromatography.

Disadvantages: (1) Slower. It takes 1 to 5 minutes to create a useful deposit. It also takes time to switch to a new analyte since the capillary must be thoroughly cleared of any leftover sample from the last measurement before spraying can start. (2) Salt adducts are a problem and desalting of the matrix and the sample is usually needed to eliminate cationization signals. (3) Extra equipment is required, along with training. (4) It involves the use of dangerous high voltages.

Aerospray (pneumatic spraying) has been suggested as an alternative sample spraying method. Recent results have demonstrated high degree of reproducibility for this sample preparation technique (Wilkins et al., 1998). Homogeneous thin films can be easily made, with good spot-to-spot and sample-to-sample reproducibility.

The potential exists to combine both techniques, using aerospray for the nebulization and an electric field to control solvent evaporation and droplet size.

j. Matrix Pre-Coated Targets

The use of matrix-precoated targets for the MALDI analysis of peptides and proteins has been investigated by several research groups. It is easy to realize the advantages of a sample preparation method reduced to the straightforward addition of a single drop of undiluted sample to a precoated target spot. Such a method would not only be faster and more sensitive than the ones described before, but it would also offer the opportunity to directly interface the MALDI sample preparation to the output of LC and CE columns.

Early efforts described the use of a pneumatic sprayer to fast-evaporate a thin matrix-only layer on a MALDI target (Kochling and Biemann, 1995). The microcrystalline films were very stable and long-lived and provided adequate MALDI spectra for peptides and small proteins.

Most other efforts have focused on the development of thin-layer matrix-precoated membranes. Particular attention has been dedicated to the choice of membrane material. Some of the options that have been tested (with varying results) include: nylon, PVDF, nitrocellulose, anion- and cation-modified cellulose and regenerated cellulose. Particularly encouraging results, in terms of sensitivity and quality of spectra, were obtained by Zhang and Caprioli (1996) for regenerated cellulose dialysis membrane. Their membrane precoating procedure provided results comparable to dried-droplet method for peptides and small proteins under 25 KDa. Heavier proteins (>25 KDa) gave poorer results, presumably due to the limited amount of matrix available in the precoated membranes and/or the inability to form protein doped microcrystals.

It has been observed that using nitrocellulose in a sample preparation for MALDI-TOF MS of peptides can increase ion yields (Preston et al., 1993). Mass spectrometry and optical microscopy results suggest that the nitrocellulose addition modifies the crystallization of the matrix-analyte solution to allow more even coverage over the sample surface.

Hutchens (1993) developed a sample preparation technique they called Surface-Enhanced Neat Desorption (SEND) in which energy-absorbing-molecules were bound to substrates to provide chemically modified surfaces capable of desorbing “neat” analyte ions. The results were very encouraging, but the technique was never mainstreamed into the general MALDI methodology.

B. Two Dimensional Gel Electrophoresis

Two-dimensional (2-D) gel electrophoresis of a sample will generate a protein profile for that sample based on the size and charge of the proteins in the sample. The profile can then be used to identify the presence or absence of a particular protein in sample, as well as identify whether the amount of that protein is changed. In addition a protein's position or location in a profile may also be altered by other chemical alteration of the protein, such as postranslational processing. The present invention concerns identifying one or more markers of prostate cancer or pre-cancerous based on differences in protein profiles of a paired sample (from the same patient), and based on accumulated data generated from the differences in profiles of multiple paired samples. For exemplary methods of one- (1-D) and two-dimensional (2-D) gel electrophoresis see U.S. Pat. No. 5,639,656; Tsai et al, 1984; Wada et al., 1985).

II. Nucleic Acid Compositions

Also contemplated by the present invention are nucleic acids encoding reproter genes, prostate marker proteins and fragments thereof.

Certain embodiments of the present invention involve the synthesis and/or mutation of at least one isolated nucleic acid molecule, such as recombinant expression vectors encoding all or part of the amino acid sequences of reporter genes or prostate marker proteins. Embodiments of the invention also involve the creation and use of recombinant host cells through the application of DNA recombinant technology, that express one or more reporter peptides or polypeptides. The nucleic acid compositions can, for example, be used in a bioassay for determining the androgen responsiveness of a cell, tissue, or biopsy.

Because of the degeneracy of the genetic code, many other nucleic acids also may encode a given reporter or prostate marker protein. For example, four different three-base codons encode the amino acids alanine, glycine, proline, threonine and valine, while six different codons encode arginine, leucine and serine. Only methionine and tryptophan are encoded by a single codon. A table of amino acids and the corresponding codons is presented herein for use in such embodiments.

In order to generate any nucleic acid encoding a reporter or prostate marker protein, one need only refer to the preceding codon table. Substitution of the natural codon with any codon encoding the same amino acid will result in a distinct nucleic acid that encodes a peptide, polypeptide or a variant thereof. As a practical matter, this can be accomplished by site-directed mutagenesis of an existing gene or de novo chemical synthesis of one or more nucleic acids.

The preceding observations regarding codon selection, site-directed mutagenesis and chemical synthesis apply with equal force to the discussion of substitutional mutants. Normally, substitutional mutants are generated by site-directed changes in the nucleic acid designed to alter one or more codons of the coding sequence.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

TABLE 1

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moiety. Preferred alkyl (e.g., alkyl, caboxyalkyl, etc.) moieties comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like. A table non-limiting, purine and pyrimidine derivatives and analogs is also provided herein below.

TABLE 2
Purine and Pyrmidine Derivatives or Analogs

| Abbr. | Modified base description |
|---|---|
| ac4c | 4-acetylcytidine |
| Chm5u | 5-(carboxyhydroxylmethyl)uridine |
| Cm | 2′-O-methylcytidine |
| Cmnm5s2u | 5-carboxymethylamino-methyl-2-thioridine |
| Cmnm5u | 5-carboxymethylaminomethyluridine |
| D | Dihydrouridine |
| Fm | 2′-O-methylpseudouridine |
| Gal q | Beta,D-galactosylqueosine |
| Gm | 2′-O-methylguanosine |
| I | Inosine |
| I6a | N6-isopentenyladenosine |
| m1a | 1-methyladenosine |
| m1f | 1-methylpseudouridine |
| m1g | 1-methylguanosine |
| m1I | 1-methylinosine |
| m22g | 2,2-dimethylguanosine |
| m2a | 2-methyladenosine |
| m2g | 2-methylguanosine |
| m3c | 3-methylcytidine |
| m5c | 5-methylcytidine |
| m6a | N6-methyladenosine |
| m7g | 7-methylguanosine |
| Mam5u | 5-methylaminomethyluridine |
| Mam5s2u | 5-methoxyaminomethyl-2-thiouridine |
| Man q | Beta,D-mannosylqueosine |
| Mcm5s2u | 5-methoxycarbonylmethyl-2-thiouridine |
| Mcm5u | 5-methoxycarbonylmethyluridine |
| Mo5u | 5-methoxyuridine |
| Ms2i6a | 2-methylthio-N6-isopentenyladenosine |
| Ms2t6a | N-((9-beta-D-ribofuranosyl-2-methylthiopurine- |
| | 6-yl)carbamoyl)threonine |
| Mt6a | N-((9-beta-D-ribofuranosylpurine-6-yl)N-methyl- |
| | carbamoyl)threonine |
| Mv | Uridine-5-oxyacetic acid methylester |
| o5u | Uridine-5-oxyacetic acid (v) |
| Osyw | Wybutoxosine |
| P | Pseudouridine |
| Q | Queosine |
| s2c | 2-thiocytidine |
| s2t | 5-methyl-2-thiouridine |
| s2u | 2-thiouridine |
| s4u | 4-thiouridine |
| T | 5-methyluridine |
| t6a | N-((9-beta-D-ribofuranosylpurine-6- |
| | yl)carbamoyl)threonine |
| Tm | 2′-O-methyl-5-methyluridine |
| Um | 2′-O-methyluridine |
| Yw | Wybutosine |
| X | 3-(3-amino-3-carboxypropyl)uridine, (acp3)u |

A nucleobase may be comprised in a nucleoside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moiety which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moiety replacing phosphodiester backbone moiety used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136 which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

In a non-limiting example, one or more nucleic acid analogs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges).

E. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 2001, incorporated herein by reference).

F. Purification of Nucleic Acids

A nucleic acid may be purified on polyacrylamide gels, cesium chloride centrifugation gradients, or by any other means known to one of ordinary skill in the art (see for example, Sambrook et al., 2001 incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

G. Nucleic Acid Segments

In certain embodiments, the nucleic acid is a nucleic acid segment. As used herein, the term “nucleic acid segment,” are fragments of a nucleic acid, such as for non-limiting example, those that encode only part of a peptide or polypeptide sequence. Thus, a “nucleic acid segment” may comprise any part of a gene sequence, of from about 2 nucleotides to the full length of the peptide- or polypeptide-encoding region.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created:
n to n+y
   where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

In a non-limiting example, nucleic acid segments may contain up to 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 nucleotides. Nucleic acid segments may also contain up to 10,000, 20,000, 30,000, 50,000, 100,000, 250,000, 500,000, 750,000, to 1,000,000 nucleotides in length, as well as constructs of greater size, up to and including chromosomal sizes are contemplated for use in the present invention. Furthermore, nucleic acids, including expression constructs, may comprise at least 5, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 4000, or 5000 contiguous nucleic acid residues or nucleotides from SEQ ID NO: 1, 3, 5, 7, 9, 11, 13, 15, 17, or 21.

In certain embodiments, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or such like. The term “cDNA” refers to that portion of a gene that is transcribed.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared containing about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc.; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc. Such constructs may be implemented and used with respect to SEQ ID NO: 1, 3, 5, 7, 9, 11, 13, 15, 17, or 21.

The term “functionally equivalent codon” is used herein to refer to codons that encode the same amino acid, such as the six codons for arginine and serine, and also refers to codons that encode biologically equivalent amino acids. For optimization of expression of genes in human cells, the codons are shown in Table 3 in preference of use from left to right. Thus, the most preferred codon for alanine is thus “GCC”, and the least is “GCG” (see Table 3, below). Codon usage for various organisms and organelles can be found at the website www.kazusa.or.jp/codon/, incorporated herein by reference, allowing one of skill in the art to optimize codon usage for expression in various organisms using the disclosures herein. Thus, it is contemplated that codon usage may be optimized for other animals, as well as other organisms such as a prokaryote (e.g., an eubacteria, an archaea), an eukaryote (e.g., a protist, a plant, a fungi, an animal), a virus and the like, as well as organelles that contain nucleic acids, such as mitochondria, chloroplasts and the like, based on the preferred codon usage as would be known to those of ordinary skill in the art.

TABLE 3
Preferred Human DNA Codons

| | |
|---|---|
| Amino Acids | Codons |

| | | | | | | | | |
|---|---|---|---|---|---|---|---|---|
| Alanine | Ala | A | GCC | GCT | GCA | GCG | | |
| Cysteine | Cys | C | TGC | TGT | | | | |
| Aspartic acid | Asp | D | GAC | GAT | | | | |
| Glutamic acid | Glu | E | GAG | GAA | | | | |
| Phenylalanine | Phe | F | TTC | TTT | | | | |
| Glycine | Gly | G | GGC | GGG | GGA | GGT | | |
| Histidine | His | H | CAC | CAT | | | | |
| Isoleucine | Ile | I | ATC | ATT | ATA | | | |
| Lysine | Lys | K | AAG | AAA | | | | |
| Leucine | Leu | L | CTG | CTC | TTG | CTT | CTA | TTA |
| Methionine | Met | M | ATG | | | | | |
| Asparagine | Asn | N | AAC | AAT | | | | |
| Proline | Pro | P | CCC | CCT | CCA | CCG | | |
| Glutamine | Gln | Q | CAG | CAA | | | | |
| Arginine | Arg | R | CGC | AGG | CGG | AGA | CGA | CGT |
| Serine | Ser | S | AGC | TCC | TCT | AGT | TCA | TCG |
| Threonine | Thr | T | ACC | ACA | ACT | ACG | | |
| Valine | Val | V | GTG | GTC | GTT | GTA | | |
| Tryptophan | Trp | W | TGG | | | | | |
| Tyrosine | Tyr | Y | TAC | TAT | | | | |

It will also be understood that amino acid sequences or nucleic acid sequences may include additional residues, such as additional N- or C-terminal amino acids or 5′ or 3′ sequences, or various combinations thereof, and yet still be essentially as set forth in one of the sequences disclosed herein, so long as the sequence meets the criteria set forth above, including the maintenance of biological protein, polypeptide or peptide activity where expression of a proteinaceous composition is concerned. The addition of terminal sequences particularly applies to nucleic acid sequences that may, for example, include various non-coding sequences flanking either of the 5′ and/or 3′ portions of the coding region or may include various internal sequences, i.e., introns, which are known to occur within genes.

The nucleic acids of the present invention encompass biologically functional equivalent reporters, prostate marker polypeptides, polypeptides, or peptides. Such sequences may arise as a consequence of codon redundancy or functional equivalency that are known to occur naturally within nucleic acid sequences or the proteins, polypeptides or peptides thus encoded. Alternatively, functionally equivalent proteins, polypeptides or peptides may be created via the application of recombinant DNA technology, in which changes in the protein, polypeptide or peptide structure may be engineered, based on considerations of the properties of the amino acids being exchanged. Changes designed by man may be introduced, for example, through the application of site-directed mutagenesis techniques as discussed herein below, e.g., to introduce improvements or alterations to the antigenicity of the protein, polypeptide or peptide, or to test mutants in order to examine polypeptide(s) or peptide(s) activity at the molecular level.

Fusion proteins, polypeptides or peptides may be prepared, e.g., where the coding regions are aligned within the same expression unit with other proteins, polypeptides or peptides having desired functions. Non-limiting examples of such desired functions of expression sequences include purification or immunodetection purposes for the added expression sequences, e.g., proteinaceous compositions that may be purified by affinity chromatography or the enzyme labeling of coding regions, respectively.

Encompassed by the invention are nucleic acid sequences encoding peptides or fusion peptides, such as, for example, peptides of from 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, to 100 amino acids in length, including such numbers of contiguous amino acids.

As used herein an “organism” may be a prokaryote, eukaryote, virus and the like. As used herein the term “sequence” encompasses both the terms “nucleic acid” and “proteanaceous composition.” As used herein, the term “proteinaceous composition” encompasses the terms “protein,” “polypeptide” and “peptide.” As used herein “artificial sequence” refers to a sequence of a nucleic acid not derived from sequence naturally occurring at a genetic locus, as well as the sequence of any proteins, polypeptides or peptides encoded by such a nucleic acid. A “synthetic sequence”, refers to a nucleic acid or proteinaceous composition produced by chemical synthesis in vitro, rather than enzymatic production in vitro (i.e., an “enzymatically produced” sequence) or biological production in vivo (i.e., a “biologically produced” sequence).

H. Vectors and Expression Constructs

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Sambrook et al., 2001 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operable linked coding sequence in a particular host cell. In addition to control sequences that govern transcription and translation, vectors and expression vectors may contain nucleic acid sequences that serve other functions as well and are described infra.

In order to express a reporter gene or a peptide or polypeptide marker of the invention, it is necessary to provide a gene in an expression vehicle. Similarly to express a peptide or polypeptide, or an antisense transcript, it is necessary to provide a cDNA in an expression vehicle. The appropriate nucleic acid can be inserted into an expression vector by standard subcloning techniques. For example, an E. coli or baculovirus expression vector is used to produce recombinant polypeptide in vitro. The manipulation of these vectors is well known in the art. In one embodiment, the protein is expressed as a fusion protein with β-gal, allowing rapid affinity purification of the protein. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6×His system (Qiagen, Chatsworth, Calif.).

Some of these fusion systems produce recombinant protein bearing only a small number of additional amino acids, which are unlikely to affect the functional capacity of the recombinant protein. For example, both the FLAG system and the 6×His system add only short sequences, both of which are known to be poorly antigenic and which do not adversely affect folding of the protein to its native conformation. Other fusion systems produce proteins where it is desirable to excise the fusion partner from the desired protein. In another embodiment, the fusion partner is linked to the recombinant protein by a peptide sequence containing a specific recognition sequence for a protease. Examples of suitable sequences are those recognized by the Tobacco Etch Virus protease (Life Technologies, Gaithersburg, Md.) or Factor Xa (New England Biolabs, Beverley, Mass.).

Recombinant bacterial cells, for example E. coli, are grown in any of a number of suitable media, for example LB, and the expression of the recombinant polypeptide induced by adding IPTG to the media or switching incubation to a higher temperature. After culturing the bacteria for a further period of between 2 and 24 hours, the cells are collected by centrifugation and washed to remove residual media. The bacterial cells are then lysed, for example, by disruption in a cell homogenizer and centrifuged to separate the dense inclusion bodies and cell membranes from the soluble cell components. This centrifugation can be performed under conditions whereby the dense inclusion bodies are selectively enriched by incorporation of sugars such as sucrose into the buffer and centrifugation at a selective speed.

If the recombinant protein is expressed in the inclusion bodies, as is the case in many instances, these can be washed in any of several solutions to remove some of the contaminating host proteins, then solubilized in solutions containing high concentrations of urea (e.g. 8M) or chaotropic agents such as guanidine hydrochloride in the presence of reducing agents such as β-mercaptoethanol or DTT (dithiothreitol).

Under some circumstances, it may be advantageous to incubate the polypeptide for several hours under conditions suitable for the protein to undergo a refolding process into a conformation which more closely resembles that of the native protein. The refolding process can be monitored, for example, by SDS-PAGE or with antibodies which are specific for the native molecule (which can be obtained from animals vaccinated with the native molecule isolated from parasites). Following refolding, the protein can then be purified further and separated from the refolding mixture by chromatography on any of several supports including ion exchange resins, gel permeation resins or on a variety of affinity columns.

There also are a variety of eukaryotic vectors that provide a suitable vehicle in which recombinant polypeptide can be produced. HSV has been used in tissue culture to express a large number of exogenous genes as well as for high level expression of its endogenous genes. For example, the chicken ovalbumin gene has been expressed from HSV using an α promoter. Herz and Roizman (1983). The lacZ gene also has been expressed under a variety of HSV promoters.

As used herein, the term “complementary” means nucleic acid sequences that are substantially complementary over their entire length and have very few base mismatches. For example, nucleic acid sequences of fifteen bases in length may be termed complementary when they have a complementary nucleotide at thirteen or fourteen positions with only a single mismatch. Naturally, nucleic acid sequences which are “completely complementary” will be nucleic acid sequences which are entirely complementary throughout their entire length and have no base mismatches.

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. Thus, in certain embodiments, expression includes both transcription of a gene and translation of a RNA into a gene product. In other embodiments, expression only includes transcription of the nucleic acid.

In preferred embodiments, the nucleic acid is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, with the exception of the androgen regulatable promoters described herein, so long as it is capable of expressing the nucleic acid in the targeted cell. Thus, where a human cell is targeted, it is preferable to position the nucleic acid coding region adjacent to and under the control of a promoter that is capable of being expressed in a human cell.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters which are well-known in the art to achieve expression of a transgene is contemplated as well, provided that the levels of expression are sufficient for a given purpose. Tables 4 and 5 list several elements/promoters which may be employed, in the context of the present invention, to regulate the expression of a transgene. This list is not exhaustive of all the possible elements involved but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression of a transgene. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 4
PROMOTER

Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
α_{1-Antitrypsin}
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

TABLE 5

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rI)X |
| | poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle |
| | Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |

Use of the baculovirus system will involve high level expression from the powerful polyhedron promoter.

One will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal and the bovine growth hormone polyadenylation signal, convenient and known to function well in various target cells. Also contemplated as an element of the expression cassette is a terminator. These elements can serve to enhance message levels and to minimize read through from the cassette into other sequences.

A specific initiation signal also may be required for efficient translation of coding sequences. These signals include the ATG initiation codon and adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements (Bittner et al., 1987).

In various embodiments of the invention, the expression construct may comprise a virus or engineered construct derived from a viral genome. The ability of certain viruses to enter cells via receptor-mediated endocytosis and to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986) and adeno-associated viruses. Retroviruses also are attractive gene transfer vehicles (Nicolas and Rubenstein, 1988; Temin, 1986) as are vaccinia virus (Ridgeway, 1988) and adeno-associated virus (Ridgeway, 1988). Such vectors may be used to (i) transform cell lines in vitro for the purpose of expressing proteins of interest or (ii) to transform cells in vitro or in vivo to provide therapeutic polypeptides in a gene therapy scenario.

HSV is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

1. Viral Vectors

Viral vectors are a kind of expression construct that utilizes viral sequences to introduce nucleic acid and possibly proteins into a cell. The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Vector components of the present invention may be a viral vector that encode one or more candidate substance or other components such as, for example, an immunomodulator or adjuvant for the candidate substance. Non-limiting examples of virus vectors that may be used to deliver a nucleic acid of the present invention are described below.

a. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the candidate substances of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

C. Retroviral Vectors

Retroviruses have promise as an antigen delivery vectors in vaccines of the candidate substances due to their ability to integrate their genes into the host genome, transferring a large amount of foreign genetic material, infecting a broad spectrum of species and cell types and of being packaged in special cell-lines (Miller, 1992).

In order to construct a vaccine retroviral vector, a nucleic acid (e.g., one encoding a reporter gene of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

e. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

2. Vector Delivery and Cell Transformation/Transfection

Suitable methods for nucleic acid delivery for transformation/transfection of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (WO 94/09699 and WO 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

3. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. It is contemplated that samples used in methods of the invention can be transfected with an expression construct like the host cells. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in or removed from at least one organism. In certain embodiments, the organism may be, but is not limited to, a prokaryote (e.g., a eubacteria, an archaea) or an eukaryote (e.g., human), as would be understood by one of ordinary skill in the art (see, for example, webpage phylogeny._arizona._edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F—, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. Coli LE392 are particularly contemplated as host cells for phage viruses.

Examples of eukaryotic host cells for replication and/or expression of a vector include, but are not limited to, primary cells derived from a biological sample(s), HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

It is an aspect of the present invention that the nucleic acid compositions described herein may be used in conjunction with a host cell. For example, a host cell (e.g., a primary human prostate epithelial cell) may be transfected with an expression construct comprising an androgen regulatable promoter operatively linked to a reporter gene.

III. Antibodies Reactive to Marker Proteins.

In another aspect, the present invention includes antibody compositions that are immunoreactive with a prostate marker protein or a polypeptide(s) identified by the present invention, or any portion thereof. Antigen refers to any polypeptide or peptide that may be used to generate antibodies. In still other embodiments, an antigen of the invention may be used to produce antibodies and/or antibody compositions. Antibodies may be specifically or preferentially reactive to prostate marker protein or a polypeptide(s) identified by the present invention, or any portion thereof. Antibodies reactive to prostate marker proteins includes antibodies reactive to polypeptides or polynucleotides encoding polypeptides, as identified by protein profile comparison as described herein. The antibodies may be polyclonal or monoclonal and produced by methods known in the art. The antibodies may also be monovalent or bivalent. An antibody may be split by a variety of biological or chemical means. Each half of the antibody can only bind one antigen and, therefore, is defined monovalent. Means for preparing and characterizing antibodies are well known in the art (see, e.g., Howell and Lane, 1988, which is incorporated herein by reference).

Peptides corresponding to one or more antigenic determinants of a prostate marker protein or a polypeptide(s) identified by the present invention, or any portion thereof of the present invention may be prepared in order to produce an antibody. Such peptides should generally be at least five or six amino acid residues in length, will preferably be about 10, 15, 20, 25 or about 30 amino acid residues in length, and may contain up to about 35 to 50 residues or so. Synthetic peptides will generally be about 35 residues long, which is the approximate upper length limit of automated peptide synthesis machines, such as those available from Applied Biosystems (Foster City, Calif.). Longer peptides also may be prepared, e.g., by recombinant means. In other methods full or substantially full length polypeptides may be used to produce antibodies of the invention.

Once a peptide(s) are prepared that contain at least one or more antigenic determinants, the peptides are then employed in the generation of antisera against the polypeptide. Minigenes or gene fusions encoding these determinants also can be constructed and inserted into expression vectors by standard methods, for example, using PCR cloning methodology. The use of peptides for antibody generation or vaccination typically requires conjugation of the peptide to an immunogenic carrier protein, such as hepatitis B surface antigen, keyhole limpet hemocyanin or bovine serum albumin. Methods for performing this conjugation are well known in the art.

The antibodies used in the methods of the invention include derivatives that are modified, i.e, by the covalent attachment of any type of molecule to the antibody such that covalent attachment For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand (a other protein, etc. Any of numerous chemical modifications may be carried out by known techniques, including, but cot limited to, specific chemical cleavage, acetylation, formylation metabolic synthesis tunicamycin, etc. Additionally, the derivative may contain one or more non-classical ammo acids.

For some uses, including in vivo use of antibodies in humans and in vitro detection assays, it may be preferable to use chimeric, humanized, or human antibodies. A dimeric antibody is a molecule in which different portions of the antibody are derived from different animal species, such as antibodies having a variable region derived from a murine monoclonal antibody and a constant region derived, from a human immunoglobulin. Methods for producing chimeric antibodies are known in the art. See e.g., Gillies et al. (1989); U.S. Pat. Nos. 5,807,715; 4,816,567; and 4,816,397, which are incorporated herein by reference in their entireties. Humanized antibodies are antibody molecules from non-human species that bind the desired antigen having one or more complementarity determining regions (CDRs) from the non-human species and framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., U.S. Pat. No. 5,585,089 and Riechmann et al. (1988), which are incorporated herein by reference in their entireties. Antibodies can be humanized using a variety of techniques known in the art including, for example, CDR-grafting (EP 239,400; WO 91/09967; U.S. Pat. Nos. 5,225,539; 5,530,101 and 5,585,089), veneering or resurfacing (EP 592,106; EP 519,596; Padlan, 1991; Studnicka et al., 1994; Roguska et al., 1994), and chain shuffling (U.S. Pat. No. 5,565,332), all of which are hereby incorporated by reference in their entireties.

Completely human antibodies are particularly desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,710,111; and WO 98/46645; WO 99/50433; WO 98/24893; WO 98/16654; WO 96/34096; WO 96/33735; and WO 91/10741, each of which is incorporated herein by reference in its entirety.

Human antibodies can also be produced using transgenic mice which are incapable of expressing functional endogenous immunoglobulins, but which can express human immunoglobulin genes. For an overview of this technology for producing human antibodies, see Lonberg and Huszar (1995). For a detailed discussion of this technology for producing human antibodies and human monoclonal antibodies and protocols for producing such antibodies, see, e.g., WO 98/24893; WO 92/01047; WO 96/34096; WO 96/33735; EP 0598877; U.S. Pat. Nos. 5,413,923; 5,625,126; 5,633,425; 5,569,825; 5,661,016; 5,545,806; 5,814,318; 5,885,793; 5,916,771; and 5,939,598; which are incorporated by reference herein in their entireties. In addition, companies such as Abgenix, Inc. (Freemont, Calif.). Kirin, Inc. (Japan), Medarex (NJ) and Genpharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope (Jespers et al., 1988).

The present invention encompasses single domain antibodies, including camelized single domain antibodies (See e.g., Muyldermans et al., 2001; Nuttall et al., 2000; Reichmann and Muyldermans, 1999; WO 94/04678; WO 94/25591; and U.S. Pat. No. 6,005,079; which are incorporated herein by reference in their entireties), In one embodiment, the present invention provides single domain antibodies comprising two VH domains with modifications such that single domain antibodies are formed.

The antibodies of the invention may also be modified by the methods and coupling agents described by Davis et al. (U.S. Pat. No. 4,179,337) in order to provide compositions that can be injected into the mammalian circulatory system with substantially no immunogenic response.

In a particular embodiment, the hinge region is modified to contain only one sulfhydryl residue, prior to conjugation. For examples of these and other related methods and compositions see U.S. Pat. Nos. 6,410,690; 6,365,161; 6,303,755; 6,270,765; and 6,258,358 each of which are incorporated herein by reference.

The invention also encompasses the use of antibodies or antibody fragments comprising the amino acid sequence of any of the antibodies of the invention with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays) can be used to assay the affinity of an antibody for a particular antigen.

The invention also comprises antibodies with altered carbohydrate modifications (e.g., glycosylation, fusocylation, etc.), wherein such modification enhances antibody-mediated effector function. Carbohydrate modifications that lead to altered antibody mediated effector function are well known in the art (for example see Shields et al., 2001; Davies et al., 2001).

The present invention may provide monoclonal antibody compositions that are immunoreactive with a prostate marker protein polypeptide. As detailed above, in addition to antibodies generated against a full length prostate marker protein polypeptide, antibodies also may be generated in response to smaller constructs comprising epitopic core regions, including wild-type and mutant epitopes. In other embodiments of the invention, the use of anti-prostate marker protein single chain antibodies, chimeric antibodies, diabodies and the like are contemplated.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

However, “humanized” prostate marker protein antibodies also are contemplated, as are chimeric antibodies from mouse, rat, goat or other species, fusion proteins, single chain antibodies, diabodies, bispecific antibodies, and other engineered antibodies and fragments thereof. As defined herein, a “humanized” antibody comprises constant regions from a human antibody gene and variable regions from a non-human antibody gene. A “chimeric antibody, comprises constant and variable regions from two genetically distinct individuals. An anti-prostate marker protein humanized or chimeric antibody can be genetically engineered to comprise an prostate marker protein antigen binding site of a given of molecular weight and biological lifetime, as long as the antibody retains its prostate marker protein antigen binding site. Humanized antibodies may be prepared by using following the teachings of U.S. Pat. No. 5,889,157.

The term “antibody” is used to refer to any antibody-like molecule that has an antigen binding region, and includes antibody fragments such as Fab′, Fab, F(ab′)₂, single domain antibodies (DABs), Fv, scFv (single chain Fv), chimeras and the like. Methods and techniques of producing the above antibody-based constructs and fragments are well known in the art (U.S. Pat. Nos. 5,889,157; 5,821,333; 5,888,773, each specifically incorporated herein by reference). The methods and techniques for preparing and characterizing antibodies are well known in the art (See, e.g., Antibodies: A Laboratory Manual, Cold Spring Harbor Laboratory, 1988; incorporated herein by reference).

As also well known in the art, the immunogenicity of a particular immunogen composition can be enhanced by the use of non-specific stimulators of the immune response, known as adjuvants. Suitable molecule adjuvants include all acceptable immunostimulatory compounds, such as cytokines, toxins or synthetic compositions. In addition to adjuvants, it may be desirable to coadminister biologic response modifiers (BRM), which have been shown to upregulate T cell immunity or down-regulate suppressor cell activity.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### The Antiandrogen Flutamide Enhances Transcription in Primary Human Prostate Epithelial Cells Through an Androgen-Independent Mechanism

Initially, prostate cancer depends on androgens for growth and survival and when androgens are removed, regression occurs. These observations were made by Huggins and Hodges over 60 years ago when they reported that castration caused prostate cancer regression (Huggins and Hodges, 1941). Androgen ablation therapy (ADT) is still the most commonly used therapy for androgen-dependent (AD) prostate cancer (Brewster and Simons, 1994; Taplin et al., 1995). More recently, maximum androgen blockade was initiated where antiandrogens were administered along with GnRH analogues to simultaneously block adrenal as well as testicular androgens (Labrie et al., 1993). Kelly and Scher (1993) and Kelly et al. (1997) observed that in 30% of patients who failed androgen ablation therapy, serum prostate specific antigen (PSA) levels declined after treatment was withdrawn. Other parameters such as acid phosphatase and alkaline phosphatase levels also decreased and in some cases, the patients' hemoglobin levels rose and repeat CT scans showed a significant regression in metastatic liver lesions. These observations generated the term “antiandrogen withdrawal syndrome” and implied that antiandrogens could potentiate androgen-like activity on androgen-independent (AI) and metastatic prostate cancer.

The pathophysiology of the antiandrogen withdrawal syndrome is unclear; however, it appears that hormone refractory prostate cancer growth typically remains dependent on AR through mechanisms which are not blocked by ADT or AR antagonist treatment (Kelly et al., 1997). Androgen blockade does not necessarily imply that the AR-activated signaling pathway is silent. For example, AR amplification (Koivisto et al., 1997; Miyoshi et al., 2000; Linja et al., 2001) or increased AR stability (Gregory et al., 2001) increase receptor number which results in hypersensitivity to lowered thresholds of serum androgens. Balk (2002) reported that AR mRNA levels rose up to 70-fold in metastatic AI compared to primary prostate cancer and that AR immunostaining was nuclear in AI prostate tissue, even in patients undergoing ADT/bicalutamide treatment. Point mutations such as the T877A substitution within the ligand-binding domain of the AR in LNCaP cells (Feldman and Fledman, 2001) widen AR specificity such that estrogens, progestins, and antiandrogens can bind and transactivate the mutant AR. The AR T877A mutation has been identified in several reports on advanced AI prostate cancer (Taplin et al., 1999; Balk, 2002). Zhao (2000) reported that in the absence of androgens, cortisol transactivated an AR containing the T877A mutation along with a second L701H mutation, resulting in increased prostate cancer cell growth and PSA secretion. Most somatic mutations in the AR, observed in AI prostate tissue, collocate to discrete areas of the receptor, indicating functional selection of gain in function similar to that described above (Buchanan et al., 2001).

AR may also be activated by ligand-independent mechanisms through other signaling pathways. HER-2/neu is overexpressed in androgen independent prostate cancer (AIPC) cell lines and this cannot be blocked by the antiandrogen Casodex (Craft et al., 1999). Analyses of prostate cancer cell lines and prostate cancer tissue samples indicate that HER/neu could activate the AR through the ras/mitogen-activated protein kinase (MAPK) (Craft, 1999; Yeh et al. 1999) or AKT pathways (Wen et al., 2000). Lastly, AR co-regulators may alter AR activity in the absence of androgen, facilitating progress towards androgen-independent prostate cancer growth (Torchia et al., 1998; Gottlicher et al., 1998). In DU145 prostate cancer cells, ARA70 may enhance the agonistic activity of antiandrogens (Yeh and Chang, 1996; Miyamoto et al., 1998). ARA55 enhances androgen-regulated transcriptional activity but can also activate transcription through the antagonists 17β-estradiol and hydroxyflutamide (Fujimoto et al., 1999). Furthermore, ARA54 can activate transcription through the mutant LNCaP AR (Kang et al., 1999). A majority of recurrent prostate cancers (after androgen ablation therapy) expressed higher than normal levels of two p160 AR coactivators (Gregory et al., 2001). Other co-regulators of the AR signaling pathway include ARA24, ARA160, RB and TIFIIH, which mediate the agonist action of antiandrogens and 17β-estradiol in DU145 prostate cancer cells (Kang et al., 2001).

Previously, it has not been possible to identify the subset of patients whose tumors will respond to antiandrogen withdrawal. The present invention provides a method to routinely isolate primary human prostate epithelial (HPE) cells from individual prostate biopsies, place them in cell culture and analyze their responsiveness to androgen and antiandrogen treatment at low passage number. The prostate epithelial cell-specific probasin promoter has been previously characterized by the inventiors (Rennie et al., 1993) and a composite promoter containing two copies of the probasin androgen responsive region (ARR2PB) linked to the CAT gene resulted in high levels of androgen-regulated transgene expression in transfection assays and targeted reporter gene expression to the prostate in transgenic mice (Zhang et al., 2000). Since androgens selectively regulate ARR2PB-CAT expression, ARR2PB-CAT/AdBN adenoviral particles were generated to develop a bioassay to measure HPE response to androgen or antiandrogen treatment and to determine whether flutamide activates an androgen-selective promoter.

The androgen receptor is a potent regulator of gene transcription (Brinkman and Trapman, 1992; Kokontis et al., 1994; Shrahle et al., 1987; Ham et al., 1988). One prostate-specific gene that is exquisitely sensitive to androgen regulation is the rat probasin (PB) gene (Dodd et al., 1983; Rennie et al., 1993; Greenberg et al., 1994; Spence et al., 1989). In transient transfection studies, two distinct androgen receptor binding sites, ARBS-1 (located at position −236 to −223) and ARBS-2 (at position −140 to −117), were required for maximal androgen induction of CAT gene expression in the PC-3 human prostate cell line (Rennie et al., 1993). The region from −244 to −96 was named the PB androgen response region (ARR) and further characterization determined that: (a) neither binding site alone could induce CAT gene expression in response to androgen treatment, (b) linking 2 or 3 copies of either ARBS-1 or ARBS-2 alone did not constitute a biologically ARR, (c) a single point mutation in either ARBS prevented AR from binding to both sites and eliminated androgen-induced transcriptional activity (Kasper et al., 1994), and (d) AR selectively bound to ARBS-1 and ARBS-2 with high affinity (Kasper et al., 1999). The 5′-flanking region containing the ARR is also prostate-specific, targeting transgenes to the prostate epithelial cells in transgenic animals (Greenberg et al., 1994; Kasper et al., 1998; Masumori et al., 2001; Zhang et al., 2000). The −426 base pairs (bp) of the 5′-flanking region of PB are sufficient for prostate-specific epithelial cell expression in transgenic mouse models, although levels of transgene expression utilizing the −426 fragment were not as high as for the 12 kb large PB fragment (Zhang et al., 2000). Therefore, the composite ARR2PB promoter was developed to target high levels of prostate-specific expression to the transgenic prostate. Transgene expression was measured in all 4 lobes of the lobes of the prostate and localized to the epithelial cells of the prostate secretory glands. Moreover, castration studies showed that transgene expression could be restored to pre-castration levels with DHT treatment (Zhang et al., 2000). The ARR2PB promoter was used to generate adenoviral particles for the HPE assay and are described below. Additional methods and materials are listed below.

Construction of Viral Constructs. The androgen responsive 5′-flanking region [(−244/−96)(−286/+28) bp] of the rat probasin gene designated ARR₂PB was linked to the chloramphenicol acetyl transferase (CAT) reporter gene (Zhang et al., 2000) and subcloned into the XhoI/BglII restriction sites of pAdBN (AdenoQuest Quantum Biotechnology, Montreal, Canada). Viral particles were generated in 293 cells according to the manufacturer's protocol and titrated in LNCaP cells. Only those clones which resulted in high levels of CAT gene expression in response to androgen treatment were selected and amplified for the human prostate epithelial (HPE) cell bioassay.

The human PSE promoter (Yu et al., 1999) was amplified by PCR from LNCaP genomic DNA. The −5322 to −3742 PSA enhancer (PSE) region was amplified with the primers 5′-TCCAAAGCTTCTAGAAATCTAGCTG-3′ (SEQ ID NO:1) and 5′-ATGCCCGGGCGGGTTCCTGAG-3′ (SEQ ID NO:2) and subcloned into the HindIII/SmaI restriction sites of pPSA (plasmid provided by Colleen Nelson) containing the minimal −634 to −1 PSA promoter region. This enhancer/promoter region, designated PSE1.6, was linked to the CAT gene and subcloned into the XhoI/BglII restriction sites of pAdBN to generate PSE1.6-CAT/AdBN.

HPE Cell Culture and Bioassay. A total of 40 biopsy samples were collected from punch biopsies obtained from radical retropubic prostatectomy (28 individuals) and upon histological analyses by the pathologist, 30 biopsies were composed of benign tissue and 10 samples were composed of 5 to 95% prostate cancer (Gleason Score 3+3). Primary HPE cells were isolated and cultured according to the method of Hayward and colleagues (Hayward et al., 1987). Briefly, punch biopsies of prostate tissue were minced, tissue pieces were plated on Primaria plates (Falcon, Phoenix, AR) and HPE cells were cultured in epithelium cell selective medium (RPMI 1640 medium supplemented with 2.5% charcoal stripped, heat-inactivated FBS, 20 mM HEPES buffer, 100 units/ml penicillin, 100 μg/ml streptomycin, 0.25 μg/ml amphotericin B, 50 μg/ml gentamycin, 56 μg/ml bovine pituitary extract, 1×insulin-transferrin-selenium, 10 ng/ml epidermal growth factor and 50 ng/ml cholera toxin). HPE cells were maintained in this medium and cultured in 5% CO₂ at 37° C. The medium was changed every 2 to 4 days and HPE cells from individual patient samples were maintained and passaged for a maximum of 4 passages. Tissue fragments and aliquots of HPE cells from passage 2 were cryopreserved according to the Hayward method (Hayward, 1998), frozen and stored at −80° C. for future analysis. These tissues and cells remain viable and can be cultured for future studies.

HPE cells were plated in quadruplicate on 24-well Primaria plates (10⁵ cells/well) for infection with adenoviral particles containing the prostate epithelial cell-specific, androgen-regulated probasin promoter of rat origin (ARR₂PB) linked to the chloramphenicol acetyltransferase (CAT) reporter gene. Increasing concentrations of androgen (R1881) and/or anti-androgen (flutamide) were added to the culture medium and HPE cells were cultured for 4 days, harvested, lysed in passive lysis buffer (Promega, Madison, Wis.) and frozen at −80° C. Total protein concentrations of cell lysates were measured using the Bio-Rad Protein Assay and 20 μg protein per sample were used to determine CAT activity as described previously (Kasper et al., 1994). The human prostate cancer cell line, LNCaP, was purchased from the American Type Culture Collection (Rockville, Md.) and served as a positive control in this study.

Western Blot Analysis. Cell proteins were isolated from the phenol-ethanol supernatant after RNA extraction according to TR1 REAGENT™ protocol (Sigma, St. Louis, Mo.). Protein concentrations were determined by the Bradford Protein Assay (Bio-Rad). Thirty micrograms of total protein (50 ng for LNCaP cells) were separated on precast 4-12% NuPAGE®Novex polyacrylamide gels (Invitrogen, Carlsbad, Calif.) and transferred onto Hybond™ ECL™ Nitrocellulose membrane (Amersham Pharmacia Biotech, Uppsala, Sweden). Nonspecific binding sites were blocked with 5% Skim Milk (BD, Sparks, Md.) and the membranes were incubated with a 1:1000 dilution of rabbit polyclonal anti-AR antibody (Santa Cruz Biotechnology, Santa Cruz, Calif.), washed and subjected to 1:1000 dilution of horseradish peroxidase linked antirabbit IgG (Amersham Pharmacia Biotech, Uppsala, Sweden). Proteins were visualized by incubating the membranes for 5 minutes in ECL+plus Solution (Amersham Pharmacia Biotech, Uppsala, Sweden) and exposed to Hyperfilm™ ECL™ (Amersham Pharmacia Biotech, Uppsala, Sweden) for 1 to 2 minutes. For subsequent β-actin detection, the membrane was stripped [100 mM 2-mercaptoethanol, 2% SDS, 62.5 mM Tris-HCl for 30 min at 70° C.], washed, blocked and subjected to immunodetection as outlined above utilizing rabbit polyclonal anti-actin (1:200 dilution) as primary antibody (Santa Cruz Biotechnology, Santa Cruz, Calif.).

Reverse Transcriptase-Polymerase Chain Reaction (RT-PCR). Total RNAs from individual human prostate cancer samples were extracted using TR1 Reagent (Sigma, St. Louis, Mo.) according to the manufacturer's instructions, and RT-PCR was performed using the RT-PCR Access System (Promega, Madison, Wis.) with gene specific primers for Androgen Receptor (AR), Prostate Specific Antigen (PSA), cytokeratin 8 (CK8), cytokeratin 18 (CK18) and 18S as indicated below. The RT-PCR products were electrophoresed on 1.5% agarose gels.

TABLE 6

| | |
|---|---|
| Product | Annealing |

| | | | | |
|---|---|---|---|---|
| Gene Name | Primers | SEQ ID NO: | Length (bp) | Temp. (° C.) |

| | | | | |
|---|---|---|---|---|
| AR | 5′-AGCCCCACTGAGGAGACAACC-3′ | 3 | 350 | 60 |
| | 5′-ATCAGGGGCGAAGTAGAGCAT-3′ | 4 | | |
| PSA | 5′-CCTCACACCTAAGGACAAAGG-3′ | 5 | 414 | 60 |
| | 5′-CATTGAACCAGAGGAGTTCTTG-3′ | 6 | | |
| CK 8 | 5′-CTGAGATCTCAGAGATGAACCGGA-3′ | 7 | 497 | 60 |
| | 5′-TTCCCATCACGTGTCTCGATCT-3′ | 8 | | |
| CK18 | 5′-GACACCAATATCACACGACTGC-3′ | 9 | 368 | 60 |
| | 5′-TTTCTCATGGAGTCCAGGTCGA-3′ | 10 | | |
| 18S | CAAGAACGAAAGTCGGAGGTTC-3′ | 11 | 488 | 60 |
| | 5′-CTGTGATGCCCTTAGATGTCC-3′ | 12 | | |

Real-Time RT-PCR Analysis. AR concentrations in cultured HPE cells were determined by real-time quantitative PCR utilizing the icycler iQ Real-Time PCR Detection System (Bio-Rad). First-strand cDNA was synthesized from 2 μg total cellular RNA with oligo(dT) primer and Super Script II reverse transcriptase (Life Technologies). The AR standard curve was generated using sequential dilutions of the human pSVAR₀ expression vector. The human 18S gene was subcloned into the pGEM-T plasmid (Promega, Madison, Wis.) and served as an internal standard. PCR reaction for HPE samples and standards were performed using SYBR Green PCR Core Reagent (PE Biosystems), followed by analysis of melting curves to validate the real-time RT-PCR data and agarose gel electrophoresis analysis of an aliquot from each RT-PCR product to monitor purity of the specific RT-PCR product. AR concentrations (in μM) were determined and standardized to the 18S product from the same sample.

AR Mutational Analysis. The ARs from HPE cells were screened for potential somatic mutations. PCR amplification of six targeted areas in which most of the prostate cancer somatic mutations collocate [2 in the AR-NTD [aa 54-92; 253-282;] and 4 in the AR-HBD [aa 654-689; 688-721; 723-738; 867-917] was performed on genomic DNA ((Buchanan et al., 2001). The following primer pairs were designed to encompass the targeted areas.

TABLE 7

| | | | | |
|---|---|---|---|---|
| Target | SEQ ID | Primer | Amino Acid | Annealing |

| | | | | | |
|---|---|---|---|---|---|
| Name | Primers | NO: | Location | Range | Temp. (° C.) |

| | | | | | |
|---|---|---|---|---|---|
| AR-I | 5′ CTT TCC AGA ATC TGT TCC AGA G 3′ | 13 | Exon | Leu54 to | 55 |
| | 5′ CCT CAT CCA GGA CCA GGT AGC C 3′ | 14 | Exon | Ser92 | |
| AR-II | 5′ GTG TGG AGG CGT TGG AGC AT 3′ | 15 | Exon | Leu253 to | 48 |
| | 5′ GAA CCT TTG CAT TCG GCC AA 3′ | 16 | Exon | Pro282 | |
| AR-IV | 5′ ACC AGC CCC ACT GAG GAG ACA A 3′ | 17 | Exon | Thr654 to | 62 |
| | 5′ TGC AAA GGA GTC GGG CTG GT 3′ | 18 | Exon | Asn689 | |
| AR-V4 | 5′ AGG TGT AGT GTG TGC TGG AC 3′ | 19 | Exon | Asp688 to | 53 |
| | 5′ CCA CTT CCC TTT TCC TTA CC 3′ | 20 | Intron | Pro721 | |
| AR-V5 | 5′ TAC CCA GAC TGA CCA CTG CC 3′ | 21 | Intron | Phe723 to | 61 |
| | 5′ AAA CAC CAT GAG CCC CAT CC 3′ | 22 | Exon | Ser738 | |
| AR-VI | 5′ GAG GCC ACC TCC TTG TCA ACC CTG 3′ | 23 | Intron | Ile867 to | 53 |
| | 5′ GGG GTG GGG AAA TAG GGT TT 3′ | 24 | Intron | End917 | |

The PCR products were sequenced utilizing an Applied Biosystems ABI Prism 377 DNA Sequencer at the Norris Cancer Center Genomics Core facility (UCS/Norris, LA) and the resulting data aligned to the AR sequence (Tilley et al., 1989) using Sequence Navigator software.

Immunohistochemical Studies. Immunoperoxidase staining was performed as follows. Briefly, cells grown on coverslips were fixed in 2% paraformaldehyde in PBS for 20 minutes at room temperature. The fixed cells were permeabilized in 0.1% Triton X-100 and non-specific binding was blocked with PBS containing 10% fetal calf serum. The primary antibodies used in this study include rabbit polyclonal antibodies directed against PSA (Dako, Carpinteria, Calif.), PSAP (Dako, Carpinteria, Calif.), and pan-cytokeratin (Santa Cruz Biotechnology, Santa Cruz, Calif.), and mouse monoclonal antibodies raised against the androgen receptor (Santa Cruz Biotechnology, Santa Cruz, Calif.), CD59 (RDI), and adipophilin (RDI). Controls were performed utilizing the corresponding blocking peptides or normal rabbit serum if blocking peptides were not available. Indirect immunoperoxidase was performed using a Vectastain kit (Vector Labs, Burlingame, Calif.) as suggested by the manufacturer.

Electron Microscopy. HPE cells cultured in 6 well culture plates were fixed in 2% glutaraldehyde in PBS for 5 minutes at room temperature. Subsequently, the cells were scraped, pelleted and further fixed for 60 minutes at room temperature. The pellets were processed for electron microscopy by standard techniques and thin and semi-thin sections were cut and stained with uranyl acetate and lead citrate and photographed on a Joel X100 microscope.

Statistical Analysis. Comparisons between flutamide suppressed (FS) and flutamide activated (FA) groups by type of sample (benign vs. tumor) and biopsy area was performed using the X₂ test or Fisher's exact test where warranted. A mixed effects analysis of variance procedure was used to test the difference between mean pre-operative PSA levels and tumor volume by groups (FS vs. FA); the mixed effects analysis of variance adjusted the test statistics for the case were there are multiple biopsies for an individual.

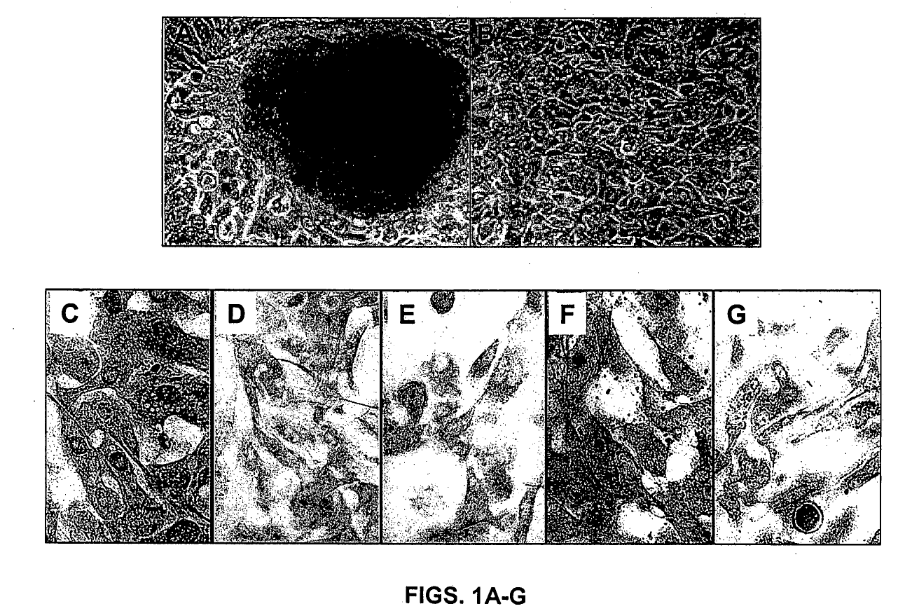

HPE Cells are Differentiated Prostatic Acinar Epithelial Cells Which Maintain Their Secretory Phenotype. A total of 40 biopsy samples (30 biopsies composed of benign tissue and 10 samples composed prostate cancer (Gleason Score 3+3) were placed into cell culture under conditions favoring epithelial cell growth as described above. HPE cells were epithelial in appearance, growing out as a monolayer of cells in a typical cobblestone pattern irrespective whether they were derived from benign or tumor tissue (FIGS. 1A-G). This growth pattern was maintained with no visible fibroblast contamination and after 5 passages, most HPE cell cultures became growth quiescent.

Long-term culture or transformation may result in loss of AR and PSA expression and secretory function (Hayward et al., 1995; Yasunaga et al., 2001a; Yasunaga et al., 2001b; Xu et al., 2003). However, several reports indicate that AR expression and secretory function can be maintained in primary cell culture (Tekur et al., 2001; Planz et al., 2001; Sinisi et al., 2002). Therefore, HPE cells were examined to determine whether they expressed AR and PSA and maintained a secretory phenotype in short-term culture. Immunoperoxidase localization of AR shows marked accumulation of AR in the nucleus of most cells (FIG. 1C) and the cytoplasm exhibits low levels of AR staining. Anti-AR antibody co-incubated with AR blocking peptide shows marked reduction in the level of staining (FIG. 1D). In addition, immunoperoxidase localization of PSA shows granular cytoplasmic staining consistent with a secretory granule staining pattern (FIG. 1E). Prostasomes are stored in membrane-bound storage vesicles in prostatic acinar epithelial cells and secreted through exocytosis or diacytosis into the glandular lumen (Yasunaga et al., 2001). HPE CD59 immunolocalization shows abundant extracellular, granular and intracellular granular staining as seen in prostasomes (FIG. 1F). Adipophilin, a protein component of lipid storage droplets, is associated with cellular differentiation and secretion (McManaman et al., 2003; Xu et al., 2003). Immunnoreactivity to adipophilin was localized in vacuolar, intracellular compartments of HPE cells, consistent with lipid droplets (FIG. 1G). Thus HPE cells in short term culture under these conditions are differentiated prostatic epithelial cells which synthesize AR and PSA and maintain their secretory phenotype as seen by prostatsome and adipophilin production.

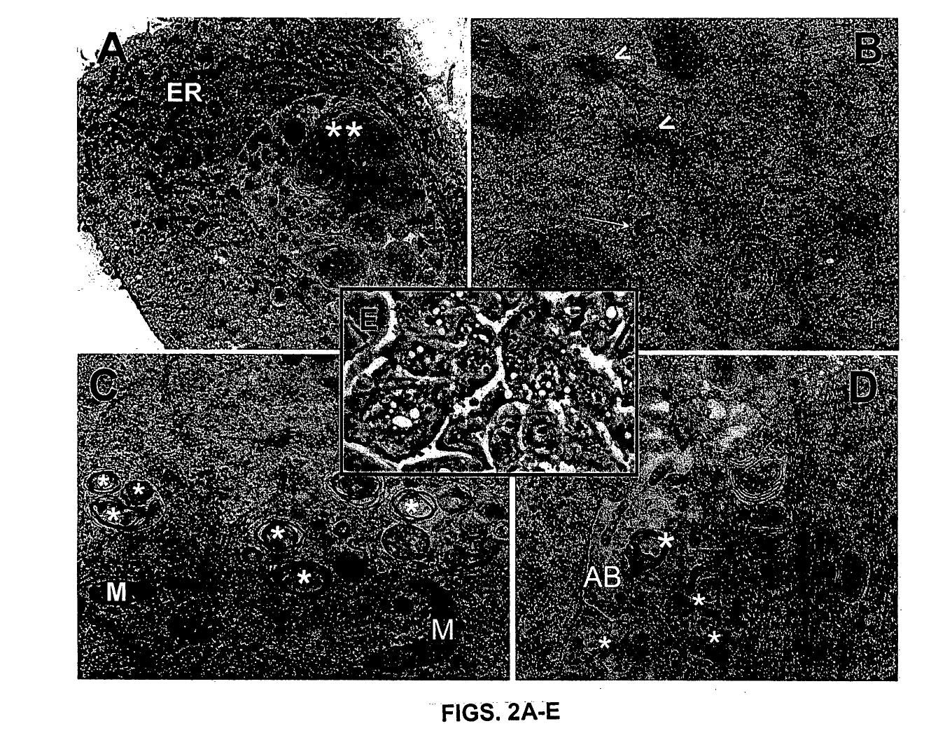

Ultrastructural Analyses. Electron microscopy (EM) analysis of HPE cells further demonstrates their epithelial characteristics. Under low magnification (FIG. 2E), HPE cells contained numerous larger and smaller highly refractile bodies. EM analysis indicated that the larger of these were secretory bodies filled with multiple tightly packed concentric membrane sheets or lamellae (FIG. 2A). The smaller secretory bodies contained loosely organized membranes, suggesting that these represented an early stage in lamellar body formation (FIG. 2C). Of particular interest is that HPE cells do not lose their brush borders which are essential for the processes of exo- and endocytosis. Secretions appear to be released at the apical brush borders of HPE cells as seen by the numerous secretory granules between two adjacent cells (FIG. 2D) and brush border vesicles (FIG. 2B). Neighboring epithelial cells are tightly bound to one another through desmosomes which form focal points of cell-cell contact and provide resilience and tensile strength to the epithelial monolayer. Desmosomes, containing characteristic attachment plaques with tonofilaments radiating from these plagues, were formed between adjacent HPE cells (FIG. 2B).

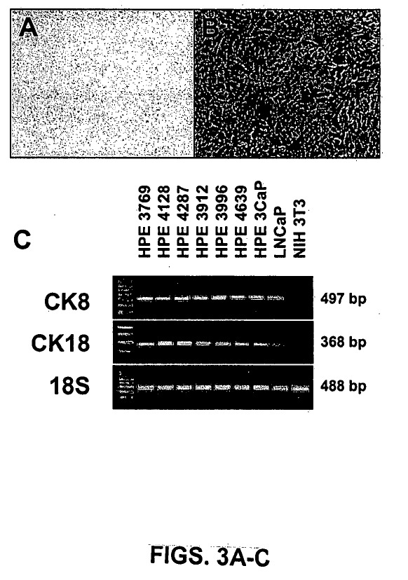

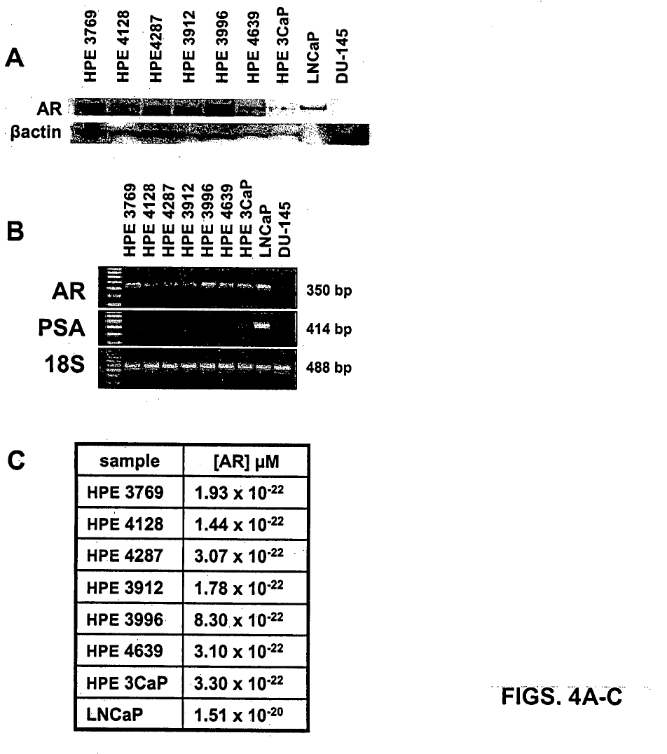

Analysis of AR in HPE Cells. Since the HPE bioassay presented below is based on androgen-regulated reporter gene expression and the PB promoter is epithelial cell specific, we determined whether HPE cells derived from patient samples were epithelial cells which continued to express AR even after multiple cell passages. The data presented utilize HPE cells after the fourth passage. Immunohistochemical analysis utilizing pan-cytokeratin (Santa Cruz Biotechnology, Santa Cruz, Calif.) determined that HPE cells expressed cytokeratins (FIG. 3B) and RT-PCR analysis confirmed that HPE cells were CK8 and CK18 positive (FIG. 3C). The cytokeratin-negative NIH-3T3 fibroblast cell line was utilized as a negative control for the RT-PCR analysis. Representative HPE cells cultured from seven individual patients expressed AR as determined by Western blot analysis (FIG. 4A) and these observations were confirmed by RT-PCR (FIG. 4B). The AR negative cell line DU-145 served as a negative control for both assays. The AR levels in LNCaP cells were 1.5×10⁻²⁰ μM as determined by real-time RT-PCR. AR concentrations in different patient samples ranged from 8.3×10⁻²² μM to 1.44×10⁻²² μM which was 18 to 104-fold lower than that measured in LNCaP cells (FIG. 4C). PSA expression has been reported to be variable in cultured cells (Yasunaga et al., 2001) and this was reflected in our data where some HPE cell cultures expressed PSA whereas others did not. Thus, our results demonstrate that AR-positive HPE cells can be utilized in transfection assays to study the effects of androgen and antiandrogen treatment on reporter gene activity.

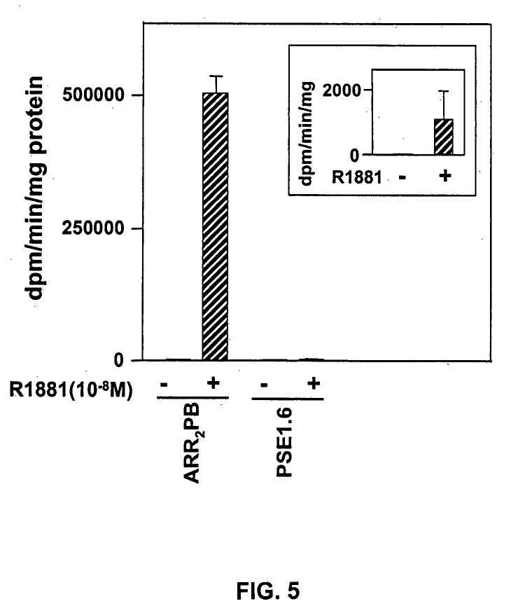

Generation and Characterization of High-Efficiency ARR₂PB-CAT Adenoviral Particles. Here, a bioassay has been developed for primary human prostate cells in culture that measures endogenous AR activity and its response to antiandrogen treatment. Since primary human cells are difficult to transfect using lipofection or electroporation methods, recombinant adenoviral particles were chosen to develop the bioassay. The PSE1.6 promoter containing the PSA enhancer and PSA core promoter regions (Yu et al., 1999) and the ARR₂PB promoter (Zhang et al., 2000) have been well-characterized. These promoters were linked to the CAT reporter gene and tested in LNCaP cells to evaluate their relative activity. Both the ARR₂PB and PSE1.6 promoters induced CAT gene activity in response to androgen treatment; however the response of the ARR₂PB promoter to androgen treatment was 500-fold greater than that induced by the PSE1.6 promoter (FIG. 5). Therefore, the ARR₂PB promoter was utilized to generate ARR₂PB-CAT/AdBN particles.

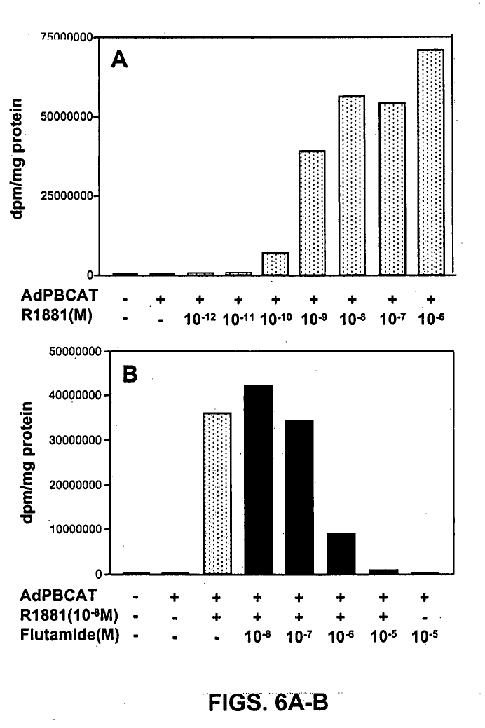

The ARR₂PB-CAT cassette was subcloned into the AdBN adenoviral vector (Adeno-Quest Kit, Quantum Biotechnologies, Inc.) and recombinant adenoviral particles were generated according to the manufacturer's protocols. Adenoviral particles containing CMV-EGFP were produced to determine transfection efficiency. All adenoviral particles were initially characterized in the androgen-dependent, LNCaP human prostate cancer cell line. Cells were plated and infected with ARR₂PB-CAT/AdBN particles (FIG. 6). No difference in basal CAT activity was seen with/without ARR₂PB-CAT/AdBN, determining that the adenoviral particles had no intrinsic biological activity in the absence of androgen (FIG. 6A). In the presence of ARR₂PB-CAT/AdBN with the addition of increasing concentrations of R1881, near maximal CAT activity was observed at 10⁻⁸ M R1881. Increasing concentrations of flutamide (10⁻⁸ M to 10⁻⁵ M) were added to the LNCaP bioassay in the presence of 10⁻⁸ M R1881 to test if flutamide could block androgen-induced CAT gene expression. Greater than 97% of the CAT activity was suppressed at 10⁻⁶ M flutamide and >99.5% was inhibited at 10⁻⁵ M flutamide (FIG. 6B). Flutamide treatment alone did not have any biological activity at any concentration tested (10⁻⁸ M to 10⁴ M tested, only 10⁻⁵ M concentration shown). This observation was also seen by Zhang and co-workers (Zhang et al., 2002) who reported that in LNCaP cells, ARR₂PB-CAT and ARR₂PB-Bax expression were not induced in response to flutamide treatment. Thus, the mutant LNCaP AR does not appear to transactivate the ARR₂PB promoter in response to flutamide treatment, whereas responsiveness to androgen treatment remains unchanged.

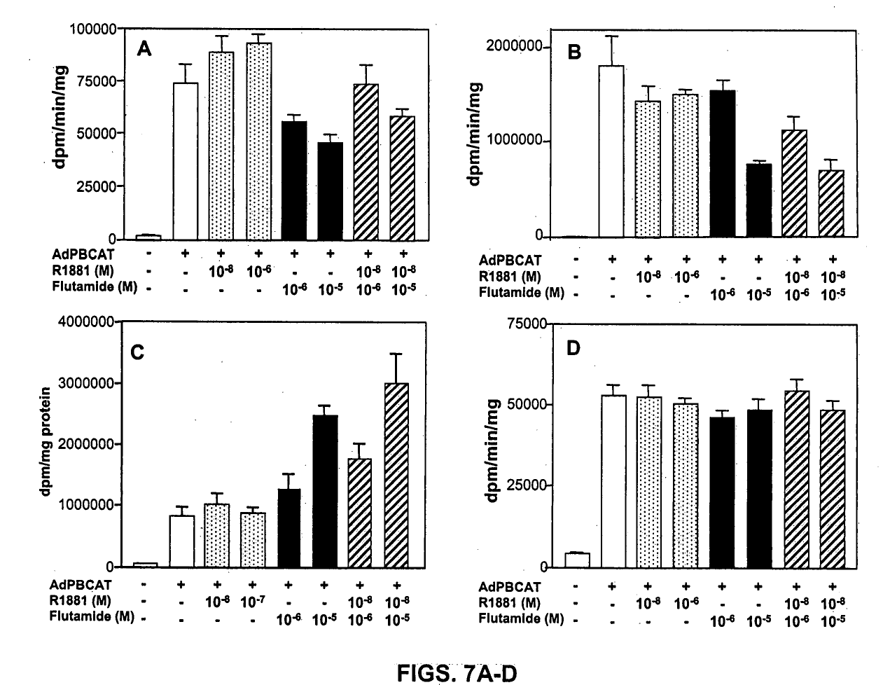

Endogenous AR activity in primary HPE cells in response to androgen and antiandrogen treatment. The composite ARR₂PB promoter is well suited for the reporter construct since it is highly sensitive to androgen and induces high levels of transgene expression in response to treatment. The following assays were performed to determine the induction of CAT expression in response to androgen and antiandrogen treatment. Representative responses are presented in FIGS. 7A-D.

In one subset of HPE cells (FIG. 7A), androgen treatment induced CAT gene expression, demonstrating that the response was still androgen-dependent. Surprisingly, high basal levels of CAT activity were measured in the presence of ARR₂PB-CAT/AdBN without androgen. This basal activity was not seen in LNCaP cells at any time (FIG. 6). Increasing concentrations of flutamide alone resulted in the partial suppression of this basal activity, suggesting that AR was still active. Flutamide in the presence of 10⁻⁸M R1881 resulted in the suppression of the androgen-induced response. These HPE cells were designated as androgen dependent/flutamide suppressed (AD/FS).

In a second subset of HPE cells (FIG. 7B), CAT gene expression was greatly elevated in the absence of androgen and the addition of 10⁻⁸ M R1881 did not increase CAT gene expression any further. These data suggest that transgene expression was already maximal under these conditions or that the activity was androgen-independent. However, adding increasing concentrations of flutamide inhibited CAT activity in a dose-dependent manner by at least 65%. These HPE cells were designated as androgen independent/flutamide suppressed (AI/FS).

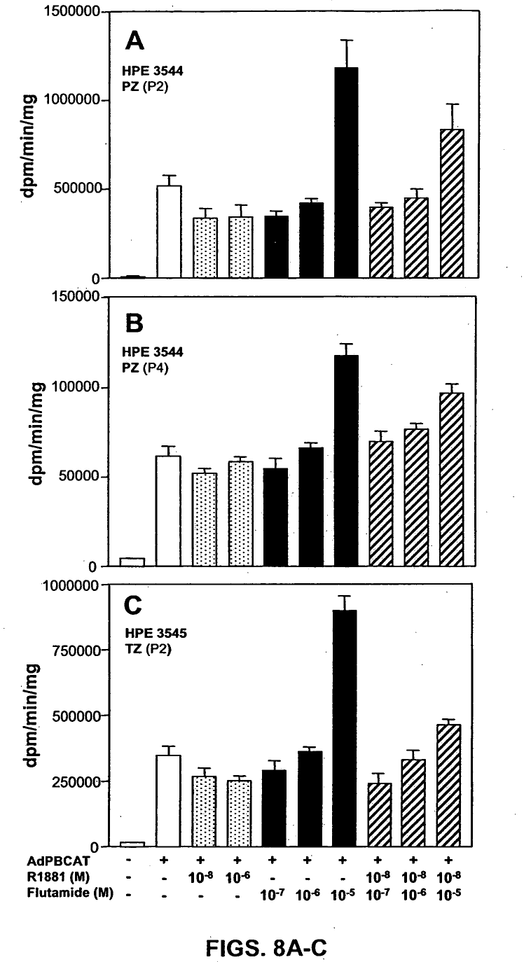

In the third subset of HPE cells (FIG. 7C), flutamide treatment alone induced CAT gene expression and in combination with androgen, the effect was greater than that seen with flutamide alone. Since androgen alone had little effect and flutamide activated ARR₂PB promoter-regulated transcription in the absence of androgen, these HPE cells were designated as androgen independent/flutamide activated (AI/FA). Since HPE cells were routinely cultured to passage 4, passage 2 HPE cells were compared with passage 4 HPE cells from the same biopsy to determine whether passage number influenced response to flutamide treatment (FIG. 8). Again, androgen alone had little effect and flutamide activated ARR₂PB promoter-regulated transcription without androgen treatment. Thus, HPE cells cultured from consecutive passages can respond to hormonal treatment in a similar manner. Finally, in the last subset of HPE cells, high levels of CAT activity were determined in the absence of androgen and the addition of androgen or flutamide did not alter this activity, indicating that they were independent/non-responsive (NR) to androgen or antiandrogen treatment (Table 8).

TABLE 8
Summary of the primary HPE bioassay in response to androgen
and antiandrogen treatment

| HPE Cell | Biological Results based on the | | |
|---|---|---|---|
| Classification | ARR₂PB-CAT assay | Frequency | % |
| AD/FS | androgen dependent/flutamide | 4/40 | 10 |
| | suppressed transcription | | |
| | CAT expression induced by androgen | | |
| | flutamide inhibits androgen-induced | | |
| | activity | | |
| AI/FS | androgen independent/flutamide | 17/40 | 42 |
| | suppressed transcription | | |
| | CAT expression not induced by | | |
| | androgen treatment | | |
| | flutamide still inhibits androgen-induced | | |
| | activity | | |
| AI/FA | androgen independent/flutamide | 14/40 | 35 |
| | activated transcription | | |
| | CAT expression not induced by | | |
| | androgen treatment | | |
| | flutamide transactivates the androgen- | | |
| | responsive ARR₂PB promoter | | |
| NR | non-responsive | 5/40 | 13 |
| | high levels of basal CAT activity | | |
| | androgen/flutamide treatment do not | | |
| | activate or inhibit | | |

Lastly, the response to flutamide treatment was compared between two biopsy samples taken from different areas of the same prostate. In the examples presented in FIGS. 8A and 8C, ARR₂PB-activated CAT gene expression was elevated in the absence of androgen and adding 10⁻⁸ M R1881did not increase these levels any further. However the addition of increasing flutamide concentrations increased CAT activity in a dose-dependent manner. Thus HPE cells cultured from two distinct areas can respond to hormonal treatment in a similar manner. The same response (either flutamide activation or flutamide suppression) was observed 60% of the time, suggesting that AR activity in HPE cells derived from different areas of the same prostate was similar.

One of the mechanisms by which flutamide activates reporter gene activity is through a mutant AR (Tan et al., 1997). The observation that flutamide activated the androgen-regulated ARR₂PB promoter in the absence of androgen (Group 3) suggests that the AR may contain a mutation. Therefore, all 14 HPE cell cultures in this group were screened for potential somatic mutations. Genomic DNA was extracted from cultured HPE cells and PCR amplification was performed on six targeted areas in which most of the prostate cancer somatic mutations collocalize, 2 in the AR-NTD [aa 54-92; 253-282;] and 4 in the AR-HBD [aa 654-689; 688-721; 723-738; 867-917] (Buchanan et al., 2001). No point mutations, insertions or deletions in these 6 areas were identified in HPE cell cultures where flutamide activated ARR₂PB promoter-regulated transcription.

HPE cultures were further analyzed to determine whether the flutamide-activated response was restricted to prostate cancer samples as compared to benign samples or whether activity was dependent on location (either transitional zone (TZ) or peripheral zone (PZ)). The ARR₂PB promoter was flutamide-activated in only 2 of the 10 tumor samples tested and no response to flutamide treatment was observed in 1 sample, whereas, flutamide suppression was observed in the remaining 7 tumor samples. Thus, 70% of tumor samples tested were still responsive to antiandrogen treatment whereas in 20% of tumor samples, flutamide appeared to act as an enhancer and not an inhibitor of AR-regulated transcription. The Gleason score was 3+3 for all samples tested, indicating that this parameter did not influence the response to HPE cells to flutamide treatment. One of the 10 tumor samples was from the TZ, and flutamide suppression of transgene expression was observed in this sample.

Flutamide activation of ARR₂PB promoter-regulated CAT activity was compared to flutamide suppression in HPE cell cultures derived from benign prostate tissue. Flutamide activation of AR-regulated CAT gene expression was observed in 85% of HPE cell cultures compared to 67% in the FS group (p=0.26). Whether the sample came from the PZ or TZ did not influence biological outcome (42% verses 39% respectively). In addition, the FA group had a mean pre-operative PSA level of 15.74±6.14 SEM (p=0.33), while the FS group had a mean PSA level of 7.65±5.26 SEM. In comparing differences in tumor volume, the FA group had a mean volume of 4.24±1.61 SEM while the FS group had a mean volume of 3.20±1.36 (p=0.63). There was also no age differences in the two groups with average age in the FA group of 60.4±2.1 years and an average age for the FS group of 59.2±1.7 years).

The collective results above can be interpreted as follows. The well-studied LNCaP, PC-3 and DU-145 prostate cell lines were established from metastatic lesions. Although these cell lines are invaluable in studying advanced prostate cancer, their usefulness in studying early genetic and molecular lesions of prostate cancer is limited. The establishment of long-term HPE cell cultures from primary tumors is difficult and human cell lines isolated from prostate tissue are typically virally transformed to immortalize them. These models are limited in that viral oncogenic alterations can influence gene expression and growth characteristics in immortalized cells. In this Example, primary HPE cells from prostate biopsies were cultured for only a limited number of passages to begin studying the early changes which may allow HPE cells to progress from an androgen-dependent to antiandrogen-independent phenotype.

Morphologically, HPE cells grow in a typical cobblestone pattern, forming an epithelial sheet with desmosomes maintaining structural integrity. HPE cells do not lose their apical brush borders even after four passages and maintain a secretory phenotype as seen by prostasome and adipophilin production, numerous lamellar bodies and secretory vesicles, and brush border vesicles. Approximately 28% of HPE cell cultures produce PSA, consistent with previous reports that PSA expression may (Korenchuk et al., 2001; Iype et al., 1998; Silva et al., 2001; Webber et al., 2001; Sinisi et al., 2002) or may not (Berthon et al., 1997; Krill et al., 1997; Yasunaga et al., 2001) be expressed in primary prostate cells in culture.

Although the exact pathophysiology of the antiandrogen withdrawal syndrome is not yet clear, AR appears to be one of the factors involved in triggering the development of AIPC. Immunohistochemical analyses of human tissue samples indicate that 85% to 99% of prostatic intraepithelial neoplasia (PIN) and PCa tissues are AR positive, with AR staining predominantly in the nucleus (Magi-Galluzzi et al., 1997; Harper et al., 1998). In histological sections, AR is present in more than 50% of cells by light microscopy. In the CWR22 xenograft model, AR localization is nuclear post castration (Gregory et al., 1998), implying that it is activated and transported into the nucleus in the absence of androgen. In the present HPE cell model, immunohistochemical analysis determined that AR localization was primarily nuclear with limited cytoplasmic AR immunoreactivity. Since HPE cells are maintained in an androgen-depleted environment (i.e., charcoal-stripped serum), these conditions could mimic those of patients undergoing androgen ablation therapy.

The HPE cell bioassay was utilized to measure the function of the endogenous AR in human prostate biopsies by characterizing its ability to activate the androgen-regulated ARR₂PB promoter in response to androgen and antiandrogen treatment. Reporter gene activity fell into 4 distinct groups as summarized in Table 8. In the AD/FS group, a classical response was observed in that androgen treatment activated ARR₂PB-regulated transcription and flutamide suppressed the androgen-activated response, suggesting that AR function was intact (FIG. 8A). In the AI/FS group, however, high basal levels of transgene activity could readily by detected in the absence of androgen and androgen treatment did not further augmented this activity (FIG. 8B). Although residual androgen levels in the charcoal-stripped serum may be sufficient to transactivate the androgen-sensitive PB promoter, this activity was not observed at any time in LNCaP cells which were subjected to the same serum conditions. Interestingly, up to 65% of the activity could be blocked by flutamide treatment, indicating that this activity was, at least in part, due to transcriptional activation by AR. The remaining activity appears to be androgen/AR independent since flutamide could not suppress this activity. This observation suggests that androgen-regulated promoters could be activated by factors which may not require the binding of AR to its DNA binding site. Lee and co-workers reported that flutamide could activate the mitogen-activated protein kinase pathway in DU-145 prostate cancer cells which do not express AR (Lee et al., 2002), implying that flutamide regulates gene expression through an androgen receptor-independent pathway. Thus it is possible that a similar mechanism may regulate the emergence of the androgen-resistant/antiandrogen-stimulated phenotype seen in HPE cell cultures. Whether this mechanism is one which promotes the emergence of an androgen resistant phenotype in HPE cells remains to be established.

Androgen-resistance was also observed in the AI/FA group and in addition, flutamide treatment alone could induce CAT gene expression in a dose dependent manner (FIG. 7C). This result was observed in 35% of samples tested and suggested that in these HPE cells, flutamide could act as an agonist. These results mimic those seen in the 30% of patients who respond to withdrawal androgen ablation therapy with a decrease in serum PSA levels (Kelly et al., 1997). PSA gene expression is regulated by androgens and the observation that PSA levels rise during androgen ablation therapy implies that antiandrogens promote its expression. In this Example, a trend was observed that a sample was more likely to fall into the FA group if pre-operative PSA levels and tumor volume were higher. Thus, in AI/FA HPE cells, this data support the idea that flutamide can progress from functioning as an antagonist and repressing androgen-induced activity to acting as an agonist and stimulating AR-regulated signal transduction.

Currently, it is thought that flutamide can promote androgen-independent growth of prostate cancer cells through a mutant AR. For example, the AR T877A mutation identified in LNCaP cells (Tan et al., 1997) now responds to flutamide treatment and the AR T877A mutation has been detected in several advanced CaP cases (Taplin et al., 2000). Thus, ARs from HPE cell cultures from the AI/FA Group 3 were screened to identify potential somatic mutations. Of the six areas in which most of the prostate cancer somatic mutations collocalize, 2 were in the AR-NTD (aa 54-92; 253-282) and 4 in the AR-HBD (aa 654-689; 688-721; 723-738; 867-917) (Buchanan et al., 2001). No mutations, including the AR T877A mutation, were identified in these six areas, implying that an intact AR could regulate the flutamide-activated response. This analysis does not eliminate the possibility that as yet unidentified mutations may have occurred outside the areas analyzed.

The induction of the androgen-regulated probasin promoter by flutamide was not anticipated. In the AI/FA Group 3, HPE cell cultures were predominantly derived from tissues which were histologically benign. Furthermore, the AR in these cells did not appear to contain any somatic mutations. Thus, these data imply that early changes at the transcriptional level have already occurred before any aberrant histology is observed. In addition, 13% of HPE cells were non-responders, i.e., high basal levels of CAT activity were evident but no increase in CAT activity was determined in response to androgen or flutamide treatment. These HPE cells seem androgen-independent and resistant to flutamide treatment and correlate with the clinical observations that approximately 20% of patients do not respond to androgen ablation therapy (van der Kwast et al., 1991). The molecular basis of this androgen independent activity remains to be established.

The selection by which resistant tumor cells emerge during androgen ablation therapy remains unclear. The transfection results of the HPE bioassay suggest that antiandrogens can act as agonists in activating AR-regulated signal transduction and that those prostate epithelial cells produce factors which promote transcription and cell proliferation in an androgen-independent manner. The antiandrogen withdrawal syndrome demonstrates that prostate cancer is a continuously evolving disease, progressing from androgen-dependence to androgen-independence. The HPE cell bioassay provides a mechanism to begin studying the early cellular and molecular changes which allow the prostate epithelial cell to develop resistance and progress from an androgen-dependent to an antiandrogen-independent phenotype.

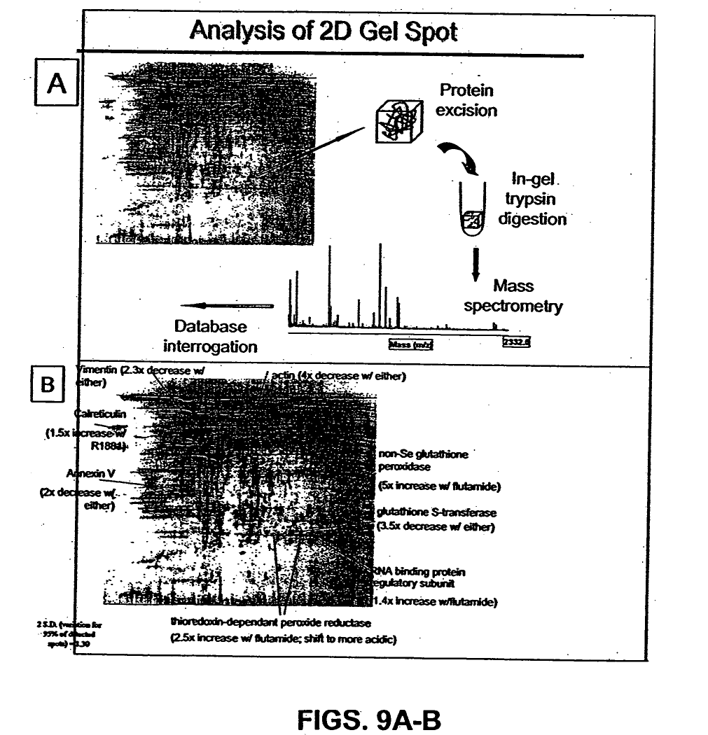

Identification of antiandrogen-regulated genes. To begin identifying proteins which are regulated by treatment, HPE cells were treated (−) hormone, (+) 10⁻⁸ M DHT or (+) 10⁻⁵ M flutamide and analyzed by 2D difference gel electrophoresis (DIGE) (FIG. 9A). In this study, 8 proteins were identified by tryptic mapping and the sequences verified by partial amino acid sequencing of individual tryptic peptides (FIG. 9B). Although flutamide is considered to bind to the AR ligand binding domain and block the AR signaling pathway, the inventors observed that flutamide could elicit its own activity. For example, expression of vimentin, actin, annexin V and glutathione S-transferase were down-regulated by both flutamide and DHT treatment in a similar manner. Furthermore, non-selenium glutathione peroxidase (NSGP), thioredoxin-dependent peroxide reductase and RNA binding protein regulatory subunit (DJ-1) were up-regulated by flutamide. Calreticulin expression decreased in the presence of flutamide and increased with DHT treatment. Both non-selenium glutathione peroxidase and thioredoxin-dependent peroxide reductase were post-translationally processed as seen by the shift to a more acidic pH. Of particular interest were calreticulin, DJ-1 and non-selenium glutathione peroxidase. Calreticulin binds to the AR DNA binding domain to block AR-activated transcription. Therefore, down-regulation of calreticulin expression would typically facilitate AR binding to the AR binding site to transactivate gene expression (Dedhar et al., 1994). Calreticulin also interacts with integrins during cell attachment and spreading (Coppolino and Dedhar, 1999) and a loss or decrease in calreticulin could potentially increase cell motility and increase HPE cell metastatic potential. The RNA binding protein regulatory subunit DJ-1 has oncogenic potential, transforming NIH3T3 cells in cooperation with ras (Nagakubo et al., 1997). DJ-1 also positively regulates AR-activated transcription by binding PIASx alpha and making it unavailable for the AR:PIASx alpha complex which functions to inhibit AR activation (Takahasi et al., 2001). Non-selenium glutathione peroxidase gene was initially identified as a KGF-regulated gene (Munz et al., 1997) and it is highly expressed in the hyperproliferative epithelium at the wound edge.

Analysis of androgen and antiandrogen treated cells by 2D difference gel electrophoreses can be used to identify proteins that interact with AR to modulate transcription, and proteins which are regulated such that they promote cell survival and growth. The identified proteins provide insight into alternative mechanisms for HPE cells to escape androgen control. Select proteins could also be used as molecular markers for the development of AIPC.

### Example 2

### MALDI MS Imaging and Molecular Fingerprinting of Prostate Biopsy Material

Androgen ablation therapy was first demonstrated by Huggins and Hodges in 1941, when they reported that castration or treatment with systemic estrogens suppressed cancer related symptoms and abnormal acid phosphatase levels in patients with advanced metastatic disease (Huggins and Hodges, 1941). Thus, androgen ablation therapy was established as the primary treatment for advanced prostate cancer. The advent of LHRH analogues allowed for chemical castration which was preferred over orchiectomy and the addition of antiandrogens provided a method to block the production of adrenal androgens, thereby allowing maximum androgen blockade. Antiandrogens have the advantage that they specifically bind the AR ligand binding domain and block activation of the AR signaling pathway. Treatment with antiandrogen is limited to the period while the tumor is still hormone responsive. Despite extensive investigation, the events occurring during the progression of androgen-dependent to hormone refractory or androgen-independent prostate cancer are poorly understood. It is believed that antiandrogen treatment influences the expression of factors which activate AR and allow androgen-independent prostate cancer cell growth. It has been shown that AR is present in most prostate cancer specimens and the prostate cancer cells in culture respond to androgen and antiandrogen treatment. Initial studies suggest that antiandrogen treatment may facilitate the development of AIPC by regulating molecules which modulate AR function. For example, calreticulin, which binds to the DNA binding domain of AR to block AR-activated transcription is down-regulated with flutamide treatment, allowing AR to interact with the AR binding site and transactivate gene expression (Dedhar et al., 1994). Calreticulin also interacts with integrins during cell attachment and spreading (Coppolino and Dedhar, 1999). Thus, repressing calreticulin levels may activate AR and facilitate cell migration. The RNA binding protein regulatory subunit DJ-1 has oncogenic potential, transforming NIH3T3 cells in cooperation with ras (Nagakubo et al., 1997). DJ-1 also positively regulates AR-activated transcription by binding PIASx alpha and making it unavailable for the AR:PIASx alpha complex which functions to inhibit AR activation (Takahashi et al., 2001). It is postulate that these factors facilitate androgen-independent prostate cancer cell growth and may serve as markers of the rate of progression to AIPC cell growth during androgen ablation therapy. HPE cells from patient biopsy samples will be treated with several antiandrogens and analyzed by 2D gel electrophoresis to determine whether the expression of calreticulin, DJ-1 and other select proteins are regulated by androgen ablation therapy.

The use of MALDI MS for profiling and identifying protein and peptides in both human biopsy material and in tissues from mouse models for human cancers has been established (Caprioli et al., 1997). Some of the techniques described herein utilize mass spectrometry (MS), particularly instrumental techniques such as matrix-assisted laser desorption ionization (MALDI) MS and electrospray ionization (ESI) MS, to identify, characterize and sequence many types of molecules. In brief, this technology utilizes a solid sample mounted on a stage, mixing or coating of the sample with a crystalline organic matrix, and a laser for the deposition of energy into the sample. A time-of-flight (TOF) analyzer is commonly used to assign mass-to-charge (m/z) ratios to the desorbed ions (Chaurand et al., 1999; Stoeckli et al., 1999).

Protein analysis by mass spectrometry (MS) requires femtomoles for determining molecular mass and a few nanograms for peptide mapping and identification (Nordhoff et al., 1999). The accuracy of MALDI MS is ±1 to 2 Daltons for proteins up to 30,000 Daltons and slightly less for proteins up to 200,000 Daltons. The Q-Star ESI TOF (Q-TOF) mass spectrometer is even more sensitive within this range. In addition, it can measure peptides with parts per million (ppm) accuracy or mass values up to 5000. The instrumental accuracy can, distinguish between 2 amino acids differing by 1 mass unit (such as asparagine and aspartic acid) in a given sequence. Therefore, the mass size (m/z) can serve as a ‘fingerprint’ to identify a given protein. Post-secondary modifications can add a distinct mass to a protein, for example, phosphorylation, methylation, acetylation and glycosylation can be identified by mass spectrometry. Therefore, even small modifications to protein structure can be determined by MS. MALDI MS has been utilized to ‘fingerprint’ the proteins in the metastatic lesions from the 12T-10 LPB-Tag mouse model for prostate cancer, and demonstrated that the lesions originated from the primary prostate tumor (Masumori et al., 2001). Therefore, since mass is highly accurate, unambiguous sequence assignments can be given to proteins that are present even at very low concentrations.

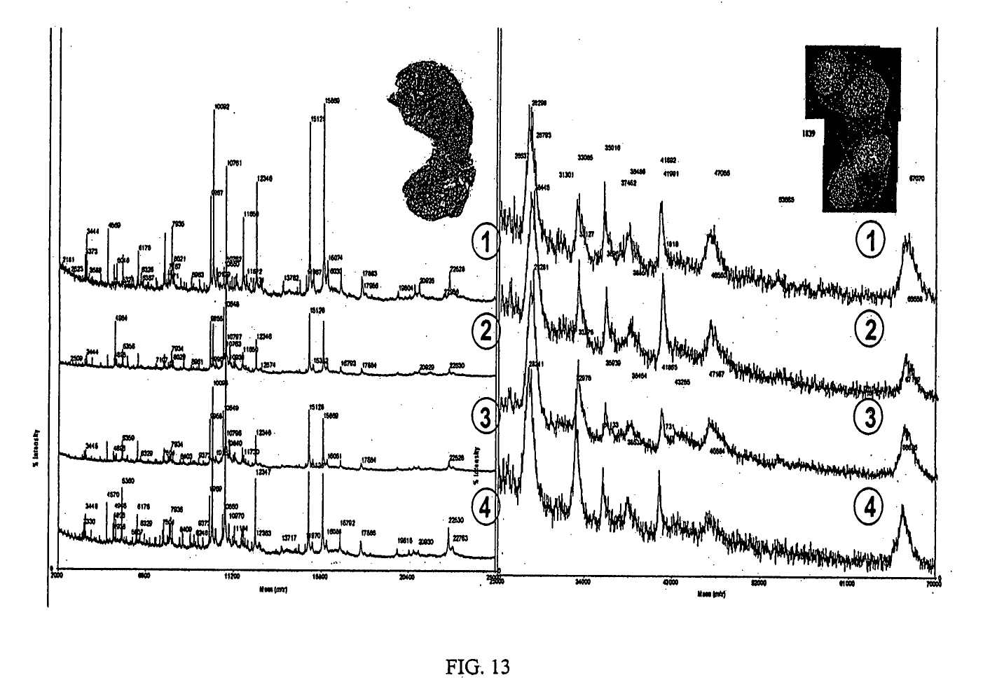

To analyze prostate biopsy material, the tissue was snap frozen and carefully placed onto a drop of optimal cutting temperature (OCT) medium which was placed onto a frozen support of OCT. This placement was crucial since fixatives interfere with MS and therefore, the tissue could not be fully embedded in the OCT as is the standard procedure for tissue sectioning. Ten micron frozen sections were cut and transferred to the metal sample plate for MS. Ten μl of crystalline organic matrix (sinaptic acid, 20 mg/ml in acetonitrile/0.05% TFA 50:50) were spotted onto the tissue, allowed to dry and the tissue sections were subjected to MS analysis. Serial sections were cut and stained with hematoxylin and eosin for histological analysis. These studies required sectioning and staining of the biopsy core and the placement of the matrix on the tissue sections for MS analysis. A coordinated effort allows for efficient analysis of the tissue and minimizes the collection of protein profiles from regions of the prostate tissue which does not contain any prostatic epithelium. Although the stroma is important in prostate cancer development, the analysis may be restricted to the epithelium from which the adenocarcinoma develops. As seen on the protein profiles, ion signals in the m/z range from 2,000 to 70,000 were observed for all of the samples (FIG. 13). The mass assignment on each of the observed ions was found to be within two mass units from spectrum to spectrum up to m/z 30,000. Overall, more than 350 distinct protein signals were detected simultaneously on this trace, suggesting that this could be a rapid screening method.

Needle biopsy cores may be analyzed by MALDI MS. Analyzing the entire biopsy core can be used to spatially localize the tumor and identify regions where protein expression has changed. Comparing the protein profiles of the needle biopsy cores with the punch biopsy samples and HPE cell bioassay results suggest that the MS protein profiles may be predictive of the biological response of HPE cells to androgen/antiandrogen treatment. Furthermore, select proteins may be molecular markers or indicators of the rate of progression to AIPC cell growth during androgen ablation therapy.

To treat primary HPE cells derived from patient prostate biopsy samples (at least one pre-treatment and at least one when PSA levels begin to rise during androgen ablation therapy) with hormones and antiandrogens to identify factors which are induced by treatment and regulate the AR signaling pathway. These factors may serve as markers of the rate of progression to AIPC cell growth during androgen ablation therapy. Several antiandrogens may be compared to determine whether the same molecules activated by flutamide treatment are also expressed in response to other antiandrogens.

Procedure for obtaining and handling biopsy samples. Briefly, prostate needle biopsies will be obtained by a physician or other appropriately trained person. A number of patients may participate in an approved program (e.g., 60 patients). Patients are enrolled in a study at the point at which the initial diagnosis of advanced prostate cancer is made (hormone naive). Primary diagnoses are established by transrectal ultrasound directed needle biopsy of the prostate. Evidence of advanced prostate cancer is defined by any of the following: PSA elevation greater than 50 ng/ml; the presence of metastatic lesions on CT scan, MRI, or bone scan, regardless of PSA level; elevation of serum acid phosphatase; biopsy proven and histologically confirmed metastatic disease. At the time of entry into a study, at least six biopsies (hormone naive) of the prostate may be taken. If the biopsy is the initial biopsy to establish the diagnosis of prostate cancer, then at least six additional biopsies may be taken for a particular study. Once the diagnosis is confirmed and the biopsies have been obtained, patients are started on androgen ablation therapy using LHRH analogues with/without antiandrogen (flutamide or bicalutamide). Hormonal therapy may be altered as necessary according to usual clinical practice. A second set of biopsies are taken at the time when serum PSA levels begin to rise. An increase in PSA levels is an indicator that the disease has progressed to become hormone refractory. The biopsy tissue is immediately minced into approximately 1 mm³ fragments and evenly distributed as follows: one third of the tissue will be frozen in liquid nitrogen and stored at −80° C. for further analysis (described below), one third will be fixed in 4% PBS-buffered formalin for histological analysis, and one third will be will be processed for HPE cell culture.

AR activity in response to androgen and antiandrogen treatment during progression to androgen-independence. For a transfection assay, HPE cells from each biopsy sample will be plated at 1×10⁵ cells per well in triplicate in 24 well plates. The HPE cells are infected with 10⁵ pfu CMV-EGFP/AdBN particles and 10¹⁰ pfu ARR2PB-CAT/AdBN and cultured for 7 days. Hormonal treatment is added at the time of viral infection and the treatment groups will be: 1) (−) virus (−) DHT; 2) (+) virus (−) DHT; 3) (+) virus (+) DHT; 4) (+) virus (−) DHT (+) flutamide and 5) (+) virus (+) DHT (+) flutamide. Alteratively, HPE cells could be treated with synthetic androgen analogues such as R1881 or with other antiandrogens, such as hydroxy-flutamide or bicalutamide. After 7 days, the medium will be collected in Eppendorf tubes, centrifuged to remove any particulate matter and stored at −80° C. for further analysis. The PSA levels will be determined by the PSA ELIZA kit (ADI). The cell pellets will be washed 2×in cold sterile PBS and stored at −80° C. until CAT activity is determined as described previously (Matusik et al., 1991). The enhanced green fluorescent protein (CMV-EGFP/AdBN, Quantum Biotechnologies) will be used to control for transfection efficiency. The PSE/PSA promoter (PSA enhancer [−4358 to −4020) fused to the proximal promoter (−640 to −1), received from Colleen Nelson] may also be operatively linked to a reporter gene such as a CAT gene and ligated into an adenoviral vector. The studies described above also will be carried out with the PSA promoter to determine the response to hormonal treatment. Other antiandrogens, circulating steroid hormones or growth factors may regulate AR-activated gene expression through other ligand-independent mechanisms. For this study, the focus will be on flutamide and bicalutamide to determine whether the same factors are regulated by these antiandrogens compared to those regulated by flutamide treatment. If the same protein profiles are revealed by 2D-gel analysis (described below), this would suggest that the development of AIPC occurs through a common mechanism.

It is anticipated that the induction of reporter gene activity and any changes in PSA levels secreted into the medium will reflect the biological activity of the AR in the sequential samples as they progress for AD to AI disease. The classification below was based on the results of 82 individual CAT assays, thus assigning a biological function to the AR. The abbreviations were carefully chosen so that they would not be confused with the abbreviation in the clinical literature, as summarized in Table 1 of Example 1.

At the time when epithelial cells are grown out of the tissue fragments and passaged up to 4 times, low passage cells will be frozen and the vials stored in liquid nitrogen. In addition, small tissue fragments are also frozen and stored in liquid nitrogen (Hayward, 1998). These tissue pieces are viable and primary cells can be cultured from them at a later date. This will generate a cell/tissue bank that contains primary human prostate cells from AD/FS, AI/FS and AI/FA phenotypes based on the biological activity of the AR as seen above. This bank will be an invaluable resource to carry out the studies described below.

AR phenotype. The changes in AR activation in response to DHT and flutamide treatment will likely reflect the progression of the HPE cells from an androgen dependent to an androgen-independent phenotype. Hormone-refractory prostate tumors still can express high levels of AR (Barrack, 1996) and cells from each group will be subjected further to Western blot analysis, RT-PCR or immunohistochemical analysis with antibodies to AR to determine whether they are AR(+) or AR(−). Since greater than 80% of primary prostate cancers have AR (Zhao et al., 2000; Trapman et al., 1990), it is anticipated that most of the samples will be AR(+). All of the HPE cells tested to date have been AR(+). As cancer of the prostate (CaP) progresses, some prostate cancer cells loose the AR. Whether ARR2PB-CAT activity will be measured in the AR(−) HPE cells will be determined. The AR(−)/CAT activity (+) cells will be stored in the cell bank to be analyzed at a later date.

Present data indicate that AR can be transactivated by factors in HPE cells in a ligand-independent manner. As well, the androgen antagonist flutamide can induce CAT gene expression in an agonistic manner. Thus, the AR may have somatic mutations such that growth factors/oncogenes/transcription factors could transactivate the AR through other signaling pathways. DNA extracted from cultured cells will be used in this analysis. First, PCR amplification of six targeted areas in which most of the prostate cancer somatic mutations collocate [3 in the AR-NTD [aa 54-78; 265-268; 502-535] and 3 in the AR-HBD [aa 668-676; 699-728; 872-908]] will be done, DNA extracted and sequenced. If no mutations are found in these areas, a more extensive mutation search will be undertaken. For this purpose, a primer pair (intron based) will be used to amplify each of the AR exons 2-7, whereas for exon 1, 5 overlapping pairs will be used to amplify this large exon whereas for exon 8 only the protein coding part will be assessed. The high fidelity polymerase pfu will be used to minimize potential PCR introduced mistakes. Sequencing will be done using the ABI large scale sequencing capability of the NCI-funded Norris Cancer Center Genomics Core facility (UCS/Norris, La.). Mutations will be verified by sequencing them in both directions. PCR products will be sequenced after agarose gel purification. About 3 pmoles of primer and 0.1 mg of PCR product are used per sequencing reaction, which is done according to manufacturer's instructions. To Multiple PCR products of the same reaction will be sequenced to verify.

The mutant AR cDNAs will be subcloned into the pCI expression vector (Promega), cotransfected with the ARR2PB-CAT reporter gene into PC-3 cells and treated with increasing concentrations of androgen (either DHT or R1881) and/or antiandrogen (flutamide, hydroxy-flutamide or bicalutamide) to determine the sensitivity of the mutant AR to hormonal treatment. As well, other steroid hormones such as glucocorticoids, estrogen and DHEA will be tested to determine if the mutant AR can transactivate CAT gene expression in the absence of androgen.

2D Difference Gel Electrophoresis analysis. Proteins are extracted from untreated, androgen- and antiandrogen-treated HPE cells and labeled with Cy-2, Cy-3 and Cy-5 respectively. Subsequently, the labeled proteins are mixed, separated by 2-D gel electrophoresis and analyzed under the appropriate wave lengths. In initial studies, the pH ranged from pH 4 to pH 7 and therefore, only proteins in that range were analyzed. Since the mature form of PSA has a pI of 7.3, it not included in that particular analysis. To cover a more complete pH range, three 2D-gel analyses will be performed for each data set; pH3-pH6, pH4-pH7, and pH7-pH 11. The proteins will be analyzed by tryptic digestion and identified in protein databases. If the tryptic map of a protein-of-interest is not in the protein database, several tryptic peptides will be sequenced by MS and the sequence used to query NCIB or Celera databases to identify the gene. Proteins from AD/FS, AI/FS and AI/FA HPE cells will be compared: a) to determine the protein expression patterns, b) to identify proteins that are specifically regulated by flutamide treatment and 3) to determine whether these patterns are similar between the different antiandrogen/steroid hormone treatments. Proteins that interact with AR (including calreticulin and DJ-1) or the AR signaling pathway will be analyzed first. When available, commercially available antibodies will be used for immunohistochemical analyses of the tissues from which the HPE cells were cultured. Alternatively, synthetic peptides may be used (based on the identified amino acid sequence) to raise antibodies to these proteins. The immunohistochemical analyses will be carried out on the formalin fixed needle biopsy samples. Since prostate epithelial cells are heterogeneous and cannot be separate in cell culture, immunohistochemical analyses will determine whether the basal or luminal epithelial proteins express the protein and whether protein expression is epithelial cell specific. Protein expression will also be correlated with tumor grade to determine whether they could be molecular markers for prostate cancer progression.

Statistical Considerations. To treat primary HPE cells derived from patient prostate biopsy samples (one pre-treatment and one when PSA levels begin to rise during androgen ablation therapy) with hormones and antiandrogens to identify factors which are induced by treatment and regulate the AR signaling pathway. The methods described herein will be used to detect expression differences in proteins that may be markers or indicators of the rate of progression of AIPC cell growth during androgen ablation therapy.

The experimental design will be a within-subject (each tissue sample from a subject gets five treatments independently) analysis of variance (randomized complete blocks design). For example, the sample size calculations are based on two-sample differences with correlation (cluster randomized t-test). Even with a high degree of within-subject correlation one will be able to detect differences in fold change of 0.5 with 80% power. With less within-subject correlation one will be able to detect even smaller differences.

Linear mixed model techniques will be used to test differences in the average fold differences between the 5 treatments. These models are of the form: Y=Xβ+Zu+e. With Xβ being the fixed part of the model, while Zu is the random portion. For the data, X is the 60×6 design matrix denoting treatment, and β is the 6×1 vector of estimated mean levels of the outcomes in each of the treatment groups and an intercept. For the random portion of the model, Z is a 300×60 design matrix denoting the 5×5 block diagonal matrix for each subject, and u is a 60×1 vector containing the within-subject variance components (this variance is assumed to be normally distributed). The 60×1 vector of errors, e, has a variance that is also assumed to be normally distributed, and describe the variability between subjects. It is also assumed that both u and e are independent. Technical details of these methods, as appled, can be found in Wolfinger and O'Connell (1993).

In a clinical study, sample size and patient dropout rate is always a potential concern. With an exemplary sample size of 60 patients, one will be able to detect a 20% change in peak height with a coefficient of variation of 45% or smaller (preliminary data show an average coefficient variation of 45% for large, small and medium peak values). This will be the case whether an exemplary target 60 subjects, or with a potential dropout rate of 20%. The sample size calculation is based on using a t-test to measure differences. Some patients are treated with LH-RH analogs alone until there is evidence of disease progression although an antiandrogen is often used during the first month of treatment. Therefore, the majority of tissue samples would not have been subjected to antiandrogen treatment. If a patient has selected to take an antiandrogen, the remaining antiandrogen in the tissue sample may inhibit DHT activation or alternatively, cause a constitutive appearance of activity. These few samples will be analyzed separately to ascertain the effects of antiandrogen treatment in the bioassay. Another potential limitation is the transfection efficiency that can be achieved in a primary cell culture; however, it has been shown that the ARR2PB-CAT/AdBN particle is highly effective in infecting the HPE cells. In fact, immunohistochemical analysis have shown that at a titre of 10¹⁰ pfu, nearly 100% of the cells stain positive with an antibody to the CAT protein (data not shown). High titre ARR2PB-luc/AdBN particles have been generated which would increase the efficiency of transfection if necessary. To limit changes that may occur when primary epithelial cells are cultured away from their microenvironment, the HPE assay is performed in cells that have only been minimally expanded (i.e., passaged 2 or 3 times) and for nuclear extract, the cells are passaged a maximum of 5 times. AR and PSA levels will be determined by immunohistochemistry, ELISA, Western blot analysis or RT-PCR as appropriate.

Prostate cancer is multifocal and any given assay may contain both prostate cancer and normal prostate epithelial cells. A method is not available by which these different cell populations can be separated. However, it is anticipate that the immunohistochemical analysis on the tissue sections (taken from the biopsy from which the HIPE cells were cultured) will determine which cell type expresses the protein of interest. This expression will be correlated with tumor grade to determine at what stage the proteins are expressed during tumor progression.

To evaluate the protein expression profiles by mass spectrometry (MS) from biopsy material collected pre-treatment and one when PSA levels begin to rise during androgen ablation therapy. It is predicted that patients with antiandrogen withdrawal syndrome will demonstrate the greatest change in protein patterns compared to patients whose cancer still responds to androgen ablation therapy. Although the inventors are interested in the overall patterns of protein expression, it is anticipate that a number of proteins may show promise as progression markers and these will be further identified and evaluated.

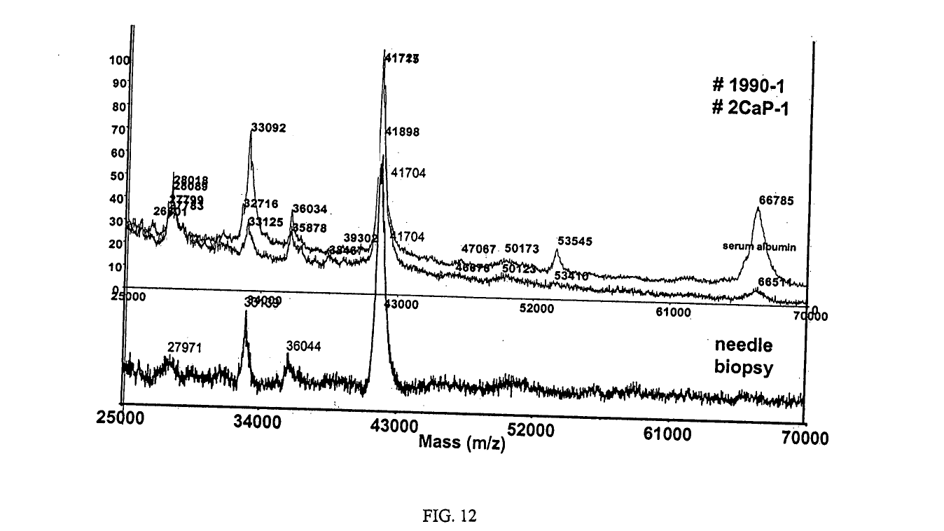

The initial results suggest that the greatest change in protein patterns occurs when flutamide activates transcription in the HPE bioassay. HPE cells from different patients respond differently such that some see flutamide as an agonist (FIG. 7) and contain proteins which are not present in HPE cells where flutamide still acts as an antiandrogen and suppresses androgen activated transcription (FIG. 9). Furthermore, the MS protein profiles from frozen needle biopsy tissue have patterns similar to those of frozen tissue sections where the biological response to antiandrogen treatment has been established by the HPE assay (FIG. 12), suggesting that these patterns could potentially predict the biological outcome when patients are in androgen ablation therapy. MS protein profiling is rapid and sensitive. From the time the needle biopsy is taken, the tissue can be processed by MS within minutes, detecting proteins at very low concentrations. Furthermore, MS protein profiling can: 1) detect hundreds of proteins simultaneously, 2) detect post-translational modifications such as phosphorylation, glycosylation and acetylation, and 3) provide protein profiles on multiple small areas on a tissue section. MS profiling can validate the proteins interacting with AR or regulating apoptosis, as well as identify new molecular markers when pre-treatment biopsy tissue is compared to post-treatment tissue obtained from patients where PSA levels have risen despite continued therapy, indicating that androgen ablation therapy is failing.

Analyses of needle biopsy samples. Needle biopsy samples that were snap-frozen at the time of tissue collection will be analyzed. These tissues are stored at −80° C. and deterioration of protein signals over time (years) is minimal. Ten micron sections are cut on a cryostat, several sections for MS analysis and several sections for H&E staining for histological analyses. For MS analysis, the tissue is desiccated and micro-droplets of sinaptic acid matrix are placed along the length of the tissue section. Protein profiles will be generated using the MALDI-TOF MS model DE-STR from Applied Biosystems (Framingham Mass.). Protein identification will be obtained by performing a protein extraction from the biopsies, fractionating the proteins by RP-HPLC, digesting the fractions containing the proteins of interest, mapping and sequencing the resulting peptides by MS, and finally, identifying the proteins using the MS information through protein and gene database searches. The accuracy of MALDI MS used for peptide sequencing is ±1 to 2 Daltons for proteins up to 30,000 Daltons. The Q-Star ESI TOF (Q-TOF) mass spectrometer will be utilized for peptide sequencing and is even more sensitive, with an accuracy ±0.1 to 0.5 Daltons. The instrumental accuracy can distinguish between 2 amino acids differing by 1 mass unit (such as asparagine and aspartic acid) in a given peptide sequence. In addition, it can distinguish proteins in the low parts per million (ppm) for peptides and proteins below MW 5000 Daltons. This is particularly advantageous for analyzing proteins which have been treated by enzymatic digestion to generate small fragments for amino acid sequencing. Therefore, the mass size (m/z) can serve as a ‘fingerprint’ to identify a given protein. Even post-secondary modifications can add a distinct mass to a protein, for example, phosphorylation, methylation and acetylation.

The protein profiles will be compared with the histological sections to determine the spatial expression of proteins along the tissue. As well, the patterns from pre-treatment tissue will be compared to those of the post-treated tissue to identify which proteins which correlate with the progression of disease in vivo. Although the exact protein levels cannot be measured from spectra to spectra, the amplitude of the peaks can be compared within a given protein profile. Therefore, imaging mass spectrometry will be used to determine semiquantitative amounts of the proteins in a pre-androgen ablation biopsy relative to those in a post-androgen ablation biopsy from the same patient. Measurement of exact protein levels is not proposed here since absolute quantitation would be difficult to establish with this technique alone. However, the inventors have demonstrated that there is a high degree of reproducibility in this technique in the preliminary data and that generation of mass spectra from needle biopsies is very feasible. Initially, proteins which show the greatest degree of change will be selected and antibodies will be raised to these to determine the immunopathology in the tissue sections. This data will provide information on the cell type (epithelial or stromal) expressing the select protein and determine any variations in expression pre- and post-androgen ablation therapy. Furthermore, the biopsy protein profiles will also be compared to the HPE bioassay results to correlate the expression of select proteins during progression in vivo (frozen sections) with the regulation of protein expression in response to androgen/antiandrogen treatment in vitro (HPE bioassay). The statistical considerations are detailed below.

Statistical Considerations. Evaluation of the protein expression profiles by mass spectrometry (MS) from biopsy material collected may be performed pre-treatment and one when PSA levels begin to rise during androgen ablation therapy.

In order to assess the relative mean peak differences the inventors will be able to detect at specific mass values (m/z) we evaluate the sample size as a function of the coefficient of variation. The coefficient of variation was chosen because in a preliminary sample of five needle biopsies the coefficient of variation was constant over mean peak heights of the lower first quartile, the middle second and third quartiles, and the largest fourth quartile; therefore, allowing for stable estimates to use as starting values. The development of this sample size calculation is described in van Belle and Martin (1993).

The inventors contemplate applying the methods of functional data analysis (FDA) to the MALDI-TOF MS data, of the type described in Ramsay and Silverman (1997) to find changes within subjects over time, as well as to compare spectra of androgen withdrawal syndrome with subjects who are continuing to respond to treatment.

MS imaging analysis of the needle biopsy tissue indicates that several hundred proteins can be profiled simultaneously, ranging from m/z 2000 to m/z 70000. Because of instrumental limitations, proteins of smaller m/z (up to 30,000) will be preferentially detected. However, larger proteins (up to 200,000) can also be detected if their concentrations are sufficient. MS imaging allows the identification of regions of similar expression and discover regions where different profiles are generated. Thus, spatial localization may be used to identify the tumor tissue from the stromal tissue and identify regions where expression of select proteins has changed. An alternative method to verify expression would be to microdissect the tumor cells by laser capture microscopy, e.g., using a Pix Cell II Laser Capture Microdissection scope. Another consideration is the evaluation of the data and what type of clustering analysis to use. The inventors recognize that an extensive amount of data will be generated using MS technology with all the samples that may be collected during various studies. It is anticipated that the information gained from comparing prostate cancer biopsy tissue pre- and post-androgen ablation therapy will provide valuable new predictive markers of prostate cancer progression. MS imaging analysis would provide a rapid method for screening clinical samples for these molecular markers.

### Example 3

### Assess the Effects of Over-Expressing Proteins Identified in Cells/Tissue which have Failed Androgen Ablation Therapy on Cell Proliferation, Hormonal Responsiveness and Invasion

The inventors contemplate determining whether select proteins promote HPE cell proliferation and/or metastasis using an invasion assay and the tissue recombination model provided by the Animal Technology Core. Factors which promote prostate cancer cell growth and/or metastasis could provide potential targets for designing selective therapy regimes based on prostate tumor characteristics.

Two-D difference gel electrophoresis has identified proteins which interact with AR or that influence cell proliferation. Androgens are essential for the development of the normal prostate and they both stimulate growth and inhibit apoptosis of prostate cancer cells. Furthermore, approximately 30% of patients respond to steroid hormone and antiandrogen withdrawal suggests that the AR is still central in the progression to AIPC. In HPE cells where flutamide acts as an agonist, the inventors have identified calreticulin and DJ-1 which directly influence AR activation. Calreticulin expression was down-regulated and DJ-1 expression was increased in response to flutamide treatment, thereby increasing AR activity. Takahashi et al. (2000) reported that DJ-1 antagonizes the repressive activity of PIASxα by binding and absorbing PIASxα from the AR:PIASxα complex, thus allowing AR activation. DJ-1 can also act as an oncogene, transforming NIH 3T3 cells (Nagakubo et al., 1997). Furthermore, DJ-1 is translocated into the nucleus during the S phase of the cell cycle, thereby promoting proliferation through the ras signaling transduction pathway (Nagakubo et al., 1997). Another protein associated with increased cell proliferation is non-selenium glutathione peroxidase (NSGP). Of all the proteins identified (FIG. 9), NSGP levels were the most highly induced by flutamide treatment. Its expression can also be regulated by keratinocyte growth factor (KGF) and it is highly expressed along the edges of hyperproliferative epithelium in wound repair, suggesting that it has growth factor activity (Munz et al., 1997). DJ-1 and NSGP represent two potential ways in which prostate cancer can become androgen-independent, namely through alterations in the interaction of AR with its co-regulators and through proteins, such as growth factors from other signaling pathways, which activate AR in the absence of androgen. It is contemplated that the following may be identified: a) proteins from the analyses of androgen/antiandrogen treated HPE cells derived from pre- and post-androgen ablation therapy and b) proteins from MS imaging analysis of frozen tissue sections pre- and post-androgen ablation therapy in the same patient. These proteins could potentially be molecular markers for rapid screening of biopsy tissue and understanding their function would provide valuable insight into designing new therapies based on tumor characteristics. In certain embodiments the potential of select proteins to promote prostate cancer cell proliferation and/or transformation and to invade the extracellular matrix will be investigated. This will be accomplished using cell culture based model systems such as the proliferation assay and the Transwell™ Permeable Support invasion assay (Cole-Palmer) and an in vivo tissue recombinant assay described below.

Proliferation assay. The sequences of DJ-1 and NSGP have been published and were used to design primers and generate corresponding cDNAs. LZRS retroviral expression vectors containing the CMV promoter, an IRES sequence and the GFP gene may be obtained. Alternatively, the prostate specific probasin promoter may be used since it is androgen-regulated. The hygromycin resistance gene will allow the selection of stably integrated cDNA/GFP expressing cells. Select cDNAs of factors identified in the flutamide-treated HPE cells and biopsies pre- and post-androgen ablation therapy will be subcloned into the LZRS vectors. HPE cells from biopsy samples expressing low/no levels of the select cDNAs will be infected and the stable transfectants selected for hygromycin resistance. The BrdU proliferation assay (Roche) will be performed according to the manufacturer's specifications to determine the effects of DJ-1 and NSGP on cell proliferation in response to constitutive overexpression (using the CMV promoter) or to androgen and antiandrogen treatment (using the probasin promoter). Cells infected with the empty vector will serve as controls. Cells will also be plated and assessed for morphological changes, such as shape change and focus formation, which suggests that transformation has occurred. If the overexpression of a select protein inhibits cell growth, then the TUNEL assay will be performed. Alternatively, BPH-1 and LNCaP cells will be tested to determine whether they express the proteins of interest. If expression levels are low/non-existent, then these cells will be used to determine the effects of overexpression on cell proliferation and transformation.

Invasion Assay. In parallel with the assays described above, invasion assays will be performed to analyze and correlate the expression of DJ-1, NSGP and select proteins on metastatic potential. The Transwell™ Permeable Support invasion assay measures the migration of tumor cells through collagen gels or Matrigel (Cole-Palmer). Briefly, 1×10⁵ cells are plated in triplicate on 8 micron permeable supports (coated with collagen or Matrigel) in 200 μl epithelial medium in the upper chamber. Medium plus 5% FBS or other chemoattractant, such as insulin like growth factor, epidermal growth factor, fibroblast growth factor, KGF, is added to the lower chamber and incubated for up to 72 hours. Cells in the upper chamber are removed and cells attached on the chemoattractant side are visualized under the appropriate wavelength (ex 488/em 509) since they express GFP. Alternatively, cells can be stained with 0.5% toluidine blue (Sigma) in H₂O for 3 to 5 min, processed in the same manner and the cells on the chemoattractant side counted. Six visual fields (1 mm² each) are counted per study and averaged, and the mean of 3 studies per treatment will be determined. HPE cells from sample 4630 can migrate through the permeable support. It is contemplated that some of the proteins, for example DJ-1, will accelerate migration and increase cell metastatic potential.

Tissue Recombinant Assay. Cell expressing proteins of interest will also be recombined with mesenchymal cells and the tissue recombinants will be implanted under the renal capsule of SCID mice. The histology of the recombinants will be analyzed. This includes the removal, processing, sectioning, and staining with appropriate antibodies of the tissue recombinants. Antibodies to DJ-1 (Stressgen Biotechnologies Corporation), calreticulin and annexin V (Santa Cruz Biotechnology, Inc.) are commercially available. Antibodies to other proteins of interest will be generated to recombinant proteins. Tissue recombinant histopathology will also be compared with the original frozen sections to determine the effects of overexpressing select proteins on tumorigenesis. The proteins identified in tissues where androgen ablation therapy has failed may promote prostate cancer progression in an in vivo assay.

The primary outcome measures are total number of BrDU cells (cell proliferation), # migrating cells (invasion), and percent normal versus abnormal (tissue recombination) between proteins which are over-expressed or hormonally regulated. Each set of protein cells (it is estimated that 5 proteins will be identified in specific aim 2) will be recombined and the recombination will be treated with one of 5 treatments, with one control.

Sample Size: A power curve for the tests in differences between Poisson means (BRDU and migrating cells) are based on a two-sided, two-sample t-test with significance level of 0.05 (standard deviation equal to the mean). With 20 recombinations per treatment one will be able to detect average count differences (with 80% power) of 6 BRDU and migrating cells between treatment groups against the control for each protein recombination. The tissue recombinant analysis will focus on the proportion of abnormal gland production. A power curve for testing the differences between proportions using a two-sided χ ²test statistic, with significance level of 0.05. With 20 samples per treatment group (per protein) one will have 80% to detect average differences in proportions of abnormal glandular production of 0.70 between treated groups and the control.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,179,337
U.S. Pat. No. 4,444,887
U.S. Pat. No. 4,470,111
U.S. Pat. No. 4,659,774
U.S. Pat. No. 4,682,195
U.S. Pat. No. 4,683,202
U.S. Pat. No. 4,684,611
U.S. Pat. No. 4,797,368
U.S. Pat. No. 4,816,397
U.S. Pat. No. 4,816,567
U.S. Pat. No. 4,816,571
U.S. Pat. No. 4,952,500
U.S. Pat. No. 4,959,463
U.S. Pat. No. 5,139,941
U.S. Pat. No. 5,141,813
U.S. Pat. No. 5,214,136
U.S. Pat. No. 5,223,618
U.S. Pat. No. 5,225,539
U.S. Pat. No. 5,264,566
U.S. Pat. No. 5,302,523
U.S. Pat. No. 5,322,783
U.S. Pat. No. 5,378,825
U.S. Pat. No. 5,384,253
U.S. Pat. No. 5,413,923
U.S. Pat. No. 5,428,148
U.S. Pat. No. 5,446,137
U.S. Pat. No. 5,464,765
U.S. Pat. No. 5,466,786
U.S. Pat. No. 5,470,967
U.S. Pat. No. 5,530,101
U.S. Pat. No. 5,545,806
U.S. Pat. No. 5,554,744
U.S. Pat. No. 5,563,055
U.S. Pat. No. 5,563,055
U.S. Pat. No. 5,569,825
U.S. Pat. No. 5,574,146
U.S. Pat. No. 5,580,859
U.S. Pat. No. 5,585,089
U.S. Pat. No. 5,585,089
U.S. Pat. No. 5,589,466
U.S. Pat. No. 5,591,616
U.S. Pat. No. 5,602,240
U.S. Pat. No. 5,602,244
U.S. Pat. No. 5,610,042
U.S. Pat. No. 5,610,289
U.S. Pat. No. 5,614,617
U.S. Pat. No. 5,623,070
U.S. Pat. No. 5,625,126
U.S. Pat. No. 5,633,425
U.S. Pat. No. 5,639,656
U.S. Pat. No. 5,645,897
U.S. Pat. No. 5,652,099
U.S. Pat. No. 5,656,610
U.S. Pat. No. 5,661,016
U.S. Pat. No. 5,670,663
U.S. Pat. No. 5,672,697
U.S. Pat. No. 5,681,947
U.S. Pat. No. 5,700,922
U.S. Pat. No. 5,702,932
U.S. Pat. No. 5,705,629
U.S. Pat. No. 5,708,154
U.S. Pat. No. 5,714,606
U.S. Pat. No. 5,736,524
U.S. Pat. No. 5,763,167
U.S. Pat. No. 5,777,092
U.S. Pat. No. 5,780,448
U.S. Pat. No. 5,789,215
U.S. Pat. No. 5,792,847
U.S. Pat. No. 5,807,715
U.S. Pat. No. 5,814,318
U.S. Pat. No. 5,821,333
U.S. Pat. No. 5,858,988
U.S. Pat. No. 5,859,221
U.S. Pat. No. 5,872,232
U.S. Pat. No. 5,885,793
U.S. Pat. No. 5,886,165
U.S. Pat. No. 5,888,773
U.S. Pat. No. 5,889,157
U.S. Pat. No. 5,889,157
U.S. Pat. No. 5,916,771
U.S. Pat. No. 5,939,598
U.S. Pat. No. 5,945,100
U.S. Pat. No. 5,981,274
U.S. Pat. No. 5,994,136
U.S. Pat. No. 5,994,624
U.S. Pat. No. 6,005,079
U.S. Pat. No. 6,013,516
U.S. Pat. No. 6,258,358
U.S. Pat. No. 6,270,765
U.S. Pat. No. 6,303,755
U.S. Pat. No. 6,365,161
U.S. Pat. No. 6,410,690
Antibodies: A Laboratory Manual, Cold Spring Harbor Laboratory, Cold Spring Harbor Press, Cold Spring Harbor, N.Y., 1988.
Ausubel et al., In: Current Protocols in Molecular Biology, John, Wiley & Sons, Inc, New York, 1994.
Bahr et al., J. Mass. Spectrom., 32:1111, 1997.
Baichwal and Sugden, In: Gene Transfer, Kucherlapati (ed.), New York, Plenum Press, 117-148, 1986.
Balk, Urology, 60:132-138, 2002.
Barrack, Mount Sinai J. Med., 63:403-412, 1996.
Beavis and Xiang, Org. Mass Spectrom. 28:1424, 1993.
Bentzley et al., Anal. Chem., 68(13):2141-2146, 1996.
Berthon et al., Int. J. Cancer, 73:910-916, 1997.
Bittner et al., Methods in Enzymol, 153:516-544, 1987.
Blackledge et al., Anal. Chem., 67:843, 1995
Blomer et al., J. Virol., 71(9):6641-6649, 1997.
Brewster and Simons, Eur. Urol., 25:177-182, 1994.
Brinkmann and Trapman, Cancer Surveys, 14:95-111, 1992.
Brown et al., Proc. of the 45^{th} ASMS Conf. on Mass Spectrom. & Allied Tops., 1997.
Buchanan et al., Clin. Cancer Res., 7:1273-1281, 2001.
Caprioli et al., Anal. Chem., 69(23):4751-4760, 1997.
Chaurand et al., J. Am. Soc. Mass Spectrom., 10(2):91-103, 1999.
Chen and Okayama, Mol. Cell Biol., 7(8):2745-2752, 1987.
Clench et al., Rapid Commun. Mass Spectrom., 13:264, 1999.
Coppolino and Dedhar, Biochem J., 340:41-50, 1999.
Cotton et al., Proc. Natl. Acad. Sci. USA, 89(13):6094-6098, 1992.
Coupar et al., Gene, 68:1-10, 1988.
Craft et al., Nat. Med., 5:280-285, 1999.
Curiel, Nat. Immun., 13(2-3):141-64, 1994.
Davies et al., Biotech. Bioengin., 74(4):288-294, 2001.
Dedhar et al., Nature, 376:480-483, 1994.
Dodd et al., J. Biol. Chem., 1983;258:10731-10737, 1983.
Duncan et al., Rapid Commun. Mass Spectrom., 7:1090, 1993.
EP 0598877
EP 239,400
EP 266,032
EP 519,596
EP 592,106
Faulstich et al., Anal. Chem., 69(21):4349-4353, 1997.
Fechheimer et al., Proc. Natl. Acad. Sci. USA, 84:8463-8467, 1987.
Feldman and Feldman, Nat Rev Cancer, 1:34-45, 2001.
Fraley et al., Proc. Natl. Acad. Sci. USA, 76:3348-3352, 1979.
Friedmann, Science, 244:1275-1281, 1989.
Froehler et al., Nucleic Acids Res. 14(13):5399-5407, 1986.
Fujimoto et al., J. Biol. Chem., 274:8316-8321, 1999.
Gerhardt, Scand. J. Clin. Lab. Invest. Suppl., 179:31-41, 1985.
Gillies et al., J. Immunol. Methods, 125(1-2):191-202, 1989.
Glorioso et al., Mol. Biotechnol., 4(1):87-99, 1995.
Gobom et al., Anal. Chem. 72:3320, 2000.
Gopal, Mol. Cell Biol., 5:1188-1190, 1985.
Gottlicher et al., J. Mol. Med., 76:480-489, 1998.
Graham and van der Eb, Virology, 52:456-467, 1973.
Greenberg et al., Molecular Endocrinology, 8:230-239, 1994.
Gregory et al., Cancer Res., 58:5718-5724, 1998.
Gregory et al., Cancer Res., 61:2892-2898, 2001.
Gregory et al., Cancer Res., 61:4315-4319, 2001.
Grossmann et al., J. Natl. Cancer Inst., 93:1687-1697, 1002.
Grunhaus et al., Seminar in Virology, 200(2):535-546, 1992.
Guo et al., Anal. Chem. 71, 1999.
Ham et al., Nucleic Acids Research, 16:5263-5276, 1988.
Harland and Weintraub, J. Cell Biol., 101: 1094-1099, 1985.
Harper et al., J. Pathol., 186:169-177, 1998.
Hayward et al., J. Urol., 138:648-653, 1987.
Hayward, Dev. Biol. Anim., 31:14-24, 1995.
Hayward, Dev. Biol. Anim., 34:28-29, 1998.
Herz and Roizman, Cell, 33(1):145-151, 1983.
Hillenkamp and Karas, Anal. Chem., 60:2299, 1988.
Horwich et al., Virol., 64:642-650, 1990.
Huggins and Hodges, Cancer Res., 1:293-297, 1941.
Hutchens et al., Rapid Commun. Mass Spectrom. 7:5776, 1993.
Iype et al., Int. J. Oncol., 12: 257-263, 1998.
Jespers et al., Bio/technology, 12:899-903, 1988.
Jespersen et al., Anal. Chem., 71(3):660-666, 1999.
Jiang et al., J. Agric. Food Chem., 48:3305, 2000.
Kaeppler et al., Plant Cell Reports 9: 415-418, 1990.
Kanazawa et al., Biol. Pharm. Bull., 22(4):339-346, 1999.
Kaneda et al., Science, 243:375-378, 1989.
Kang et al., J. Biol. Chem., 274:8570-8576, 1999.
Kang et al., Proc. Natl. Acad. Sci. USA, 98:3018-3023, 2001.
Kasper et al., J. Biol. Chem., 269:31763-31769, 1994.
Kasper et al., Laboratory Investigation, 78:319-334, 1998.
Kasper et al., Molecular Endocrinology, 22:313-325, 1999.
Kato et al, J. Biol. Chem., 266:3361-3364, 1991.
Kelleher and Vos, Biotechniques, 17(6):1110-7, 1994.
Kelly and Scher, J. Urol., 149:607-609, 1993.
Kelly et al., Urol. Clin. North Am., 24:421-431, 1997.
Kinsel et al., Anal. Chem., 71:268, 1999.
Kochling and Biemann, Proc. of the 43rd Annual ASMS Conf. on Mass Spectrom. and Allied Topics, 1995).
Koivisto et al., Cancer Res., 57: 314-319, 1997.
Kokontis et al., Cancer Res., 54:1566-1573, 1994.
Korenchuk et al., Int. J. Cancer, 15(2):163-8, 15, 163-168, 2001.
Kornberg and Baker, DNA Replication, 2nd Ed., Freeman, San Francisco, 1992.
Krill et al., Urology, 49:981-988, 1997.
Labrie et al., Cancer, 71:1059-1067, 1993.
Laughlin et al., J. Virol., 60(2):515-524, 1986.
Lebkowski et al., Mol. Cell. Biol., 8(10):3988-3996, 1988.
Lee et al., Cancer Res., 62:6039-6044, 2002.
Li et al., Anal. Chem., 71:5451, 1999.
Li et. al., Anal. Chem., 71:1087, 1999.
Li et al., J. Am. Chem. Soc., 118:11662, 1996.
Li et al., Trends Biotechnol., 18:151, 2000.
Linja et al., Cancer Res., 61:3550-3555, 2001.
Lonberg and Huszar, Int. Rev. Immunol, 13(1):65-93, 1995
Lynn et al., Rapid Commun. Mass. Spectrom., 13(20):2022-2027, 1999.
Magi-Galluzzi et al., Mod. Pathol., 10:839-845, 1997.
Mann et al., Cell, 33:153-159, 1983.
Marie et al., Anal. Chem., 72(20):5106-5114, 2000.
Masumori et al., Cancer Res., 61, 2239-2249. 2001.
Matusik et al., In: Molecular and cellular biology of prostate cancer. NY and London: Plenum Press; 299-314, 1991.
McLaughlin et al., J. Virol., 62(6):1963-1973, 1988.
McManaman et al., J. Lipid Res., 44: 668-673, 2003.
Miketova and Schram, Mol. Biotechnol., 8(3):249-253, 1997.
Miller et al., Am. J. Clin. Oncol., 15(3):216-221, 1992.
Mirgorodskaya et al., Rapid Commun. Mass Spectrom., 14:1226, 2000.
Miyamoto et al., Proc. Natl. Acad. Sci. USA, 95:7379-7384, 1998.
Miyoshi et al., Prostate, 43:225-232, 2000.
Muddiman et al., Fres. J Anal. Chem., 354:103, 1996.
Munz et al., Biochem J., 326:579-585, 1997.
Muyldermans et al., Trends Biochem. Sci., 26:230, 2001.
Muzyczka, Curr. Top Microbiol. Immunol., 158:97-129, 1992.
Nabel et al., Science, 244(4910):1342-1344, 1989.
Nagakubo et al., Biochem Biophys Res Commun., 231:509-513, 1997.
Naldini et al., Science, 272(5259):263-267, 1996.
Nelson et al., Anal. Chem., 66:1408, 1994.
Nguyen et al., J. Chromatogr. A., 705(1):21-45, 1995.
Nicolas and Rubenstein, In: Vectors: A survey of molecular cloning vectors and their uses, Rodriguez and Denhardt (eds.), Stoneham: Butterworth, pp. 493-513, 1988.
Nicolau and Sene, Biochim. Biophys. Acta, 721:185-190, 1982.
Nicolau et al., Methods Enzymol., 149:157-176, 1987.
Nordhoffet al., Nat. Biotechnol., 17:884-888, 1999.
Nuttall et al., Cur. Pharm. Biotech., 1:253, 2000.
Omirulleh et al., Plant Mol. Biol., 21(3):415-428, 1993.
Orlando et al., Anal. Chem., 69:4716, 1997.
Owens et al., Rapid Commun. Mass Spectrom., 11:209, 1997.
Padlan, Mol. Immunol., 28(4-5):489-498, 1991.
Paskind et al., Virology, 67:242-248, 1975.
Paul and Breul, Drug Saf, 23(5):381-90, 23:381-390, 2000.
PCT Appl. WO 91/09967
PCT Appl. WO 91/10741
PCT Appl. WO 92/01047
PCT Appl. WO 94/04678
PCT Appl. WO 94/09699
PCT Appl. WO 94/25591
PCT Appl. WO 95/06128
PCT Appl. WO 96/33735
PCT Appl. WO 96/33735
PCT Appl. WO 96/34096
PCT Appl. WO 96/34096
PCT Appl. WO 98/16654
PCT Appl. WO 98/24893
PCT Appl. WO 98/24893
PCT Appl. WO 98/46645
PCT Appl. WO 99/50433
Perera et al., Rapid Commun. Mass Spectrom., 9:180, 1995.
Perreault et al., Anal. Chem., 70:5142, 1998.
Philip et al., Electrophoresis, 18:382, 1997.
Planz et al., J. Urol., 166:678-683, 2001.
Potrykus et al., Mol. Gen. Genet., 199:183-188, 1985.
Potter et al., Proc. Natl. Acad. Sci. USA, 81:7161-7165, 1984.
Preston et al., Biol. Mass Spectrom., 22:544, 1993.
Ramsay and Silverman, In: Functional Data Analysis, N.Y., Springer, 1997.
Reichmann and Muyldermans, J. Immunol. Meth., 231:25, 1999.
Rennie et al., Molecular Endocrinology, 7:23-36, 1993.
Ridgeway, In: Vectors: A survey of molecular cloning vectors and their uses, Stoneham: Butterworth, pp. 467-492, 1988.
Riechmann et al., Nature, 332, 323-327, 1988.
Rippe et al., Mol. Cell Biol., 10:689-695, 1990.
Roepstorff, EXS. 2000; 88:81-97, 2000.
Roguska et al., Proc. Natl. Acad. Sci. USA, 91(3):969-973, 1994.
Roux et al., Proc. Natl. Acad. Sci. USA, 86:9079-9083, 1989.
Russell et al., Int. J. Mass Spectrom. 182/183, 1999.
Sambrook et al., In: Molecular cloning, Cold Spring Harbor Laboratory Press, Cold Spring Harbor, N.Y., 2001.
Scheit, In: Synthesis and Biological Function, Wiley-Interscience, New York, pp. 171-172, 1980.
Schleuder et al., Anal. Chem., 71:3238, 1999.
Shields et al., J. Biol. Chem., 277(30): 26733-40, 2001.
Shrahle et al., Proc. Nat. Acad. Sci. USA, 84:7871-7875, 1987.
Silva et al., Endocr. Res., 27:153-169, 2001.
Sinisi et al., Dev. Biol. Anim., 38:165-172, 2002.
Spence et al., Proc. Nat. Acad. Sci. USA, 86:7843-7847, 1989.
Stoeckli et al., J. Am. Soc. Mass Spectrom., 10:67-71, 1999.
Stoeckli et al., Nat. Med., 7(4):493-496, 2001.
Studnicka et al., Protein Eng., 7(6):805-814, 1994.
Takach et al., J. Protein Chem., 16:363, 1997.
Takahashi et al., J. Biol. Chem., 276:37556-37563, 2001.
Tan et al., Anal. Biochem. 131:99, 1983.
Tan et al., Molecular Endocrinology, 11: 450-459, 1997.
Taplin et al., Cancer Res., 59:2511-2515, 1999.
Taplin et al., N. Engl. J. Med., 332:1393-1398, 1995.
Tekur et al., Mol. Carcinog., 30:1-13, 2001.
Temin, In: Gene Transfer, Kucherlapati (ed.), NY, Plenum Press, 149-188, 1986.
Tilley et al., Proc. Natl. Acad. Sci. USA, 86:327-331, 1989.
Torchia et al., Curr. Opin. CellBiol., 10:373-383, 1998.
Trapman et al., J. Steroid Biochem. Molec. Biol., 37:837-842, 1990.
Tratschin et al., Mol. Cell. Biol., 4:2072-2081, 1984.
Tsai et al., Clin Chem., 30(12 Pt 1):2026-2030, 1984.
Tur-Kaspa et al., Mol. Cell Biol., 6:716-718, 1986.
van Belle and Martin, American Statistician, 47:165-168, 1993.
van der Kwast et al., Int. J. Cancer, 48:189-193, 1991.
Villanueva et al., Enzyme Microb. Technol., 29:99, 1999.
Vorm et. al., Anal. Chem., 66:3281, 1994.
Wada, Prostate, 7(1):107-115, 1985
Wang et al., J. Agric. Food. Chem., 47:1549, 1999.
Wang et al., J. Agric. Food. Chem., 47:2009, 1999.
Wang et al., J. Agric. Food. Chem., 48:2807, 2000.
Wang et al., J. Agric. Food. Chem., 48:3330, 2000.
Webber et al., Prostate, 47:1-13, 2001.
Wen et al., Cancer Res., 60:6841-6845, 2000.
Wilkins et al., J. Am. Soc. Mass Spectrom. 9:805, 1998.
Wilson et al., Science, 244:1344-1346, 1989.
Wittmann et al., Biotechnol. Bioeng., 72:642, 2001.
Wolfinger and O'Connell, J. Stat. Computing and Simulation, 48:233-243, 1993.
Wong et al., Gene, 10:87-94, 1980.
Woods et al., Anal. Chem. 70:750, 1998.
Wu and Wu, J. Biol. Chem., 262:4429-4432, 1987.
Wu et al., Anal. Chem., 70:456A, 1998.
Wu et al., Biochem. Biophys. Res. Commun., 233(1):221-6, 1997.
Xu, J. Cell. Physiol., 196:131-143, 2003.
Yang et al., J. Agric. Food. Chem., 48:3990, 2000.
Yasunaga et al., Cancer Res., 61:5969-5973, 2001a.
Yasunaga et al., Oncogene. 20:8036-8041, 2001b.
Yeh and Chang, Proc. Natl. Acad. Sci. USA, 93:5517-5521, 1996.
Yeh et al., Proc. Natl. Acad. Sci. USA, 96:5458-5463, 1999.
Yu et al., Cancer Res., 59:1498-1504, 1999.
Zaluzec et al., Protein Expr. Purif., 6:109, 1995.
Zhang and Caprioli, J. Mass Spectrom., 31:690, 1996.
Zhang et al., Endocrinology, 141(12):4698-710, 141:4698-4710, 2000.
Zhang et al., Hum. Gene Ther., 13:2051-2064, 2002.
Zhao et al., Nat. Med., 6(6):703-6.6:703-706, 2000.
Zufferey et al., Nat. Biotechnol., 15(9):871-875, 1997.

1. A method for determining androgen-responsiveness of a first biological sample from a patient suspected of having cancer, comprising:
(a) producing a protein profile of the first biological sample derived from the patient suspected of having cancer;
(b) comparing the protein profile of the first biological sample with the protein profile of a second biological sample that has been characterized as being androgen-responsive and a third biological sample that has been characterized as being androgen-independent; and
(d) determining if the first biological sample is androgen-responsive.

2. The method of claim 1, wherein the cancer is prostate cancer.

3. The method of claim 1, wherein the protein profile is produced by MALDI mass spectrometry.

4. The method of claim 1, wherein the protein profile is produced by two-dimensional gel electrophoresis.

5. The method of claim 1, wherein the first biological sample is a needle biopsy.

6. The method of claim 1, wherein the first biological sample is a punch biopsy.

7. A method for determining a cancer treatment for a patient suspected of having cancer, comprising:
(a) producing a protein profile of a first biological sample from the patient;
(b) comparing the protein profile of the first biological sample with the protein profiles of a set of biological samples for which androgen-responsiveness has been determined; and
(d) determining a treatment for the patient based on whether the first biological sample is androgen-responsive.

8. The method of claim 7, wherein the cancer is prostate cancer.

9. The method of claim 7, wherein the protein profile is produced by MALDI mass spectrometry.

10. The method of claim 7, wherein the protein profile is produced by two-dimensional gel electrophoresis.

11. The method of claim 7, wherein the biological sample is a needle biopsy.

12. The method of claim 7, wherein the biological sample is a punch biopsy.

13. The method of claim 7, wherein androgen-responsiveness is determined by a method comprising:
(a) transfecting epithelial cells of the biological sample with an androgen-regulatable expression construct;
(b) exposing the transfected cells to an androgen;
(c) assaying levels of expression from the androgen-regulatable expression construct in the androgen exposed transfected cells relative to a control; and
(d) determining androgen-responsiveness of the biological sample.

14. The method of claim 13, wherein the androgen-regulatable expression construct comprises an androgen regulated promoter element operatively linked to a reporter gene.

15. The method of claim 14, wherein the androgen regulated promoter element is an androgen receptor binding site 1 (ARBS-1) and androgen receptor binding site 2 (ARBS-2) of a probasin gene.

16. The method of claim 14, wherein the reporter gene is chloramphenicol acetyltransferase, luciferase, green fluorescent protein, horse radish peroxidase.

17. The method of claim 14, wherein assaying the levels of reporter gene expression comprises nucleic acid blotting, western blotting, enzymatic assay, 2-D gel electrophoresis, or spectrometry.

18. The method of claim 13, wherein the androgen-regulatable expression construct is a viral expression construct.

19. The method of claim 18, wherein the viral expression construct an adenovirus, retrovirus, or lentivirus.

20. The method of claim 19, wherein the virus is an adenovirus.

21. A method for characterizing androgen-responsiveness of a biological sample comprising:
(a) transfecting epithelial cells of the biological sample with an androgen-regulatable expression construct;
(b) exposing the transfected cells to an androgen;
(c) assaying levels of expression from the androgen-regulatable expression construct in the androgen exposed transfected cells relative to a control; and
(d) determining androgen-responsiveness of the biological sample.

22. The method of claim 21, wherein the androgen-regulatable expression construct comprises an androgen-regulatable promoter element operatively linked to a reporter gene.

23. The method of claim 22, wherein the androgen regulated promoter element is an androgen receptor binding site 1 (ARBS-1) and androgen receptor binding site 2 (ARBS-2) of a probasin gene.

24. The method of claim 21, wherein the reporter gene is chloramphenicol acetyltransferase, luciferase, green fluorescent protein, horse radish peroxidase.

25. The method of claim 21, wherein assaying the levels of reporter gene expression comprises nucleic acid blotting, western blotting, enzymatic assay or spectrometry.

26. The method of claim 21, wherein the biological sample is a needle biopsy.

27. The method of claim 21, wherein the biological sample is a punch biopsy.

28. The method of claim 21, wherein the androgen-regulatable expression construct is a viral expression construct.

29. The method of claim 28, wherein the viral construct is further comprised in a virus.

30. The method of claim 29, wherein the virus is an adenovirus.

31. A method for determining an appropriate treatment for prostate cancer in a patient suspected of having prostate cancer comprising:
(a) transfecting epithelial cells of a biological sample from the patient suspected of having prostate cancer with an androgen-regulatable expression construct, wherein the expression construct comprises an androgen-responsive promoter element operatively linked to a reporter gene;
(b) exposing the transfected cells to an androgen;
(c) exposing the transfected cells to an antiandrogen;
(c) assaying levels of reporter gene expression in the transfected cells of (b) and (c) relative to a control;
(d) determining an appropriate treatment for the patient.

32. The method of claim 31, wherein the androgen regulated promoter element is an androgen receptor binding site 1 (ARBS-1) and androgen receptor binding site 2 (ARBS-2) of a probasin gene.

33. The method of claim 31, wherein the reporter gene is chloramphenicol acetyltransferase, luciferase, green fluorescent protein, horse radish peroxidase.

34. The method of claim 31, wherein assaying the levels of reporter gene expression comprises nucleic acid blotting, western blotting, enzymatic assay or spectrometry.

35. The method of claim 31, wherein the androgen-regulatable expression construct is a viral expression construct.

36. The method of claim 35, wherein the virus is an adenovirus.

37. A method for determining androgen-responsiveness of a biological sample from a patient, comprising:
(a) producing a protein profile of the first biological sample;
(b) producing protein profiles of a set of biological samples characterized for androgen-responsiveness by a method comprising (i) transfecting epithelial cells of a biological sample with an androgen-regulatable expression construct, (ii) exposing the transfected cells to an androgen or antiandrogen treatment; and (iii) detecting levels of reporter gene expression in the treated transfected cells relative to a control;
(c) comparing the protein profile of the first biological sample with the protein profiles of the set of biological samples for which androgen-responsiveness has been determined; and
(d) determining the androgen-responsiveness of the first biological sample.

38. The method of claim 37, wherein the protein profile is produced by MALDI mass spectrometry.

39. The method of claim 37, wherein the protein profile is produced by two-dimensional gel electrophoresis.

40. The method of claim 37, wherein the adrogen regulatable expression vector comprises an androgen regulated promoter element operatively linked to a reporter gene.

41. The method of claim 40, wherein the androgen regulated promoter element is an androgen receptor binding site 1 (ARBS-1) and androgen receptor binding site 2 (ARBS-2) of a probasin gene.

42. The method of claim 40, wherein the androgen-regulatable expression construct is a viral expression construct.

43. The method of claim 42, wherein the viral construct is further comprised in an adenovirus.

44. A method for identifying a protein marker for androgen-responsive prostate cancer comprising:
(a) transfecting epithelial cells of a first biological sample with an androgen-regulatable expression construct, wherein the expression construct comprises an androgen-responsive promoter element operatively linked to a reporter gene;
(b) exposing the transfected cells to an androgen treatment;
(c) detecting levels of reporter gene expression in the treated transfected cells relative to a control;
(d) determining androgen-responsiveness of the first biological sample;
(e) producing a protein profile of an androgen-responsive biological sample;
(f) comparing the protein profile of the androgen-responsive biological sample to a protein profile of a second androgen-independent biological sample; and
(g) identifying a protein that is diagnostic of androgen-responsive cancer.

45. The method of claim 44, wherein the cancer is prostate cancer.

46. The method of claim 44, wherein the protein profile is produced by MALDI mass spectrometry.

47. A method for identifying a protein marker for androgen-independent prostate cancer comprising:
(a) transfecting epithelial cells of a biological sample with an androgen-regulatable expression construct, wherein the expression construct comprises an androgen-responsive promoter element operatively linked to a reporter gene;
(b) exposing the transfected cells to an androgen treatment;
(c) detecting levels of reporter gene expression in the treated transfected cells relative to a control;
(d) determining androgen-responsiveness of the biological sample;
(e) producing a protein profile of an androgen-independent biological sample;
(f) comparing the protein profile of the androgen-independent biological sample to a protein profile of a second androgen-responsive biological sample; and
(g) identifying a protein that is diagnostic of an androgen-independent cancer.

48. The method of claim 47, wherein the cancer is prostate cancer.

49. The method of claim 47, wherein the protein profile is produced by MALDI mass spectrometry.

50. A protein marker for an androgen-responsive prostate cancer identified by a method comprising:
(a) transfecting epithelial cells of a biological sample with an androgen-responsive expression construct, wherein the expression construct comprises an androgen-responsive promoter element operatively linked to a reporter gene;
(b) exposing the transfected cells to an androgen treatment;
(c) detecting levels of reporter gene expression in the treated transfected cells relative to a control;
(d) producing a protein profile from transfected cells identified as being androgen-responsive;
(e) determining the identity a protein that is diagnostic of an androgen-responsive epithelial cell.

51. A protein marker for an androgen-independent prostate cancer identified by a method comprising:
(a) transfecting epithelial cells of a biological sample with an androgen-responsive expression construct, wherein the expression construct comprises an androgen-responsive promoter element operatively linked to a reporter gene;
(b) exposing the transfected cells to an androgen treatment;
(c) detecting levels of reporter gene expression in the treated transfected cells relative to a control;
(d) producing a protein profile from transfected cells identified as being androgen-independent;
(e) determining the identity a protein that is diagnostic of an androgen-independent epithelial cell.


SEQUENCE LISTING (24 sequences)

SEQ ID NO: 1 - DNA, 25 bp, Artificial Sequence
  Description of Artificial Sequence  Synthetic Primer
tccaaagcttctagaaatctagctg

SEQ ID NO: 2 - DNA, 21 bp, Artificial Sequence
  Description of Artificial Sequence  Synthetic Primer
atgcccgggcgggttcctgag

SEQ ID NO: 3 - DNA, 21 bp, Artificial Sequence
  Description of Artificial Sequence  Synthetic Primer
agccccactgaggagacaacc

SEQ ID NO: 4 - DNA, 21 bp, Artificial Sequence
  Description of Artificial Sequence  Synthetic Primer
atcaggggcgaagtagagcat

SEQ ID NO: 5 - DNA, 21 bp, Artificial Sequence
  Description of Artificial Sequence  Synthetic Primer
cctcacacctaaggacaaagg

SEQ ID NO: 6 - DNA, 22 bp, Artificial Sequence
  Description of Artificial Sequence  Synthetic Primer
cattgaaccagaggagttcttg

SEQ ID NO: 7 - DNA, 24 bp, Artificial Sequence
  Description of Artificial Sequence  Synthetic Primer
ctgagatctcagagatgaaccgga

SEQ ID NO: 8 - DNA, 22 bp, Artificial Sequence
  Description of Artificial Sequence  Synthetic Primer
ttcccatcacgtgtctcgatct

SEQ ID NO: 9 - DNA, 22 bp, Artificial Sequence
  Description of Artificial Sequence  Synthetic Primer
gacaccaatatcacacgactgc

SEQ ID NO: 10 - DNA, 22 bp, Artificial Sequence
  Description of Artificial Sequence  Synthetic Primer
tttctcatggagtccaggtcga

SEQ ID NO: 11 - DNA, 22 bp, Artificial Sequence
  Description of Artificial Sequence  Synthetic Primer
caagaacgaaagtcggaggttc

SEQ ID NO: 12 - DNA, 21 bp, Artificial Sequence
  Description of Artificial Sequence  Synthetic Primer
ctgtgatgcccttagatgtcc

SEQ ID NO: 13 - DNA, 22 bp, Artificial Sequence
  Description of Artificial Sequence  Synthetic Primer
ctttccagaatctgttccagag

SEQ ID NO: 14 - DNA, 22 bp, Artificial Sequence
  Description of Artificial Sequence  Synthetic Primer
cctcatccaggaccaggtagcc

SEQ ID NO: 15 - DNA, 20 bp, Artificial Sequence
  Description of Artificial Sequence  Synthetic Primer
gtgtggaggcgttggagcat

SEQ ID NO: 16 - DNA, 20 bp, Artificial Sequence
  Description of Artificial Sequence  Synthetic Primer
gaacctttgcattcggccaa

SEQ ID NO: 17 - DNA, 22 bp, Artificial Sequence
  Description of Artificial Sequence  Synthetic Primer
accagccccactgaggagacaa

SEQ ID NO: 18 - DNA, 20 bp, Artificial Sequence
  Description of Artificial Sequence  Synthetic Primer
tgcaaaggagtcgggctggt

SEQ ID NO: 19 - DNA, 20 bp, Artificial Sequence
  Description of Artificial Sequence  Synthetic Primer
aggtgtagtgtgtgctggac

SEQ ID NO: 20 - DNA, 20 bp, Artificial Sequence
  Description of Artificial Sequence  Synthetic Primer
ccacttcccttttccttacc

SEQ ID NO: 21 - DNA, 20 bp, Artificial Sequence
  Description of Artificial Sequence  Synthetic Primer
tacccagactgaccactgcc

SEQ ID NO: 22 - DNA, 20 bp, Artificial Sequence
  Description of Artificial Sequence  Synthetic Primer
aaacaccatgagccccatcc

SEQ ID NO: 23 - DNA, 24 bp, Artificial Sequence
  Description of Artificial Sequence  Synthetic Primer
gaggccacctccttgtcaaccctg

SEQ ID NO: 24 - DNA, 20 bp, Artificial Sequence
  Description of Artificial Sequence  Synthetic Primer
ggggtggggaaatagggttt